(19) United States
(12) Patent Application Publication
(10) Pub. No.: US 2026/0298938 A1
(43) Pub. Date: Oct. 1, 2026

(54) DETECTION COMPOSITION AND USE THEREOF

(71) Applicant: GENANS BIOTECHNOLOGY CO., LTD, Beijing (CN)

(72) Inventors: Rui LIN, Beijing (CN); Shilin ZHONG, Beijing (CN); Minmin LUO, Beijing (CN)

(21) Appl. No.: 18/996,440

(22) PCT Filed: Jul. 18, 2023
(86) PCT No.: PCT/CN2023/107980

§ 371 Date: Jan. 17, 2025

(30) Foreign Application Priority Data
Jul. 18, 2022 (WO) PCT/CN2022/106344

(51) Int. Cl. G01N 33/58; G01N 33/68

(52) U.S. Cl. CPC G01N 33/58; G01N 33/6854; G01N 2458/10

(57) ABSTRACT

Provided is a composition comprising an antibody linked with an oligonucleotide via a disulfide bond, wherein the oligonucleotide is specific for the antibody. Further provided is a kit comprising the same and the method for detecting one or more target in a sample by means of the composition.

### FIELD OF THE INVENTION

The present disclosure relates to the field of molecular biology, and particular to a composition for detecting targets and use thereof.

### BACKGROUND

Accurate and sensitive detection of target molecules in biological samples is key for biomedical research and clinical diagnostics. Owing to their ease of use, speed, and cost effectiveness, immunoassays are currently the standard and most popular methods for biomolecule detection. These methods use a primary antibody that binds selectively to a target molecule (antigen), and this antibody-antigen interaction can be visualized via a conjugated reporter on the primary antibody. More commonly, a labeled secondary antibody that is capable of recognizing primary antibodies from a same host species are used to react with the primary antibody-epitope complex to generate signals. In classic immunoassays, fluorescence or luminescence is typically utilized to indicate the abundance and the localization of target molecules. This approach allows only low multiplexing of target molecules due to the overlapping of antibody host species and reporter spectrums. The use of photo-detection as the signal acquisition mechanism also limits the throughput of immunoassays.

DNA barcoding represents a promising strategy to overcome these limitations of immunoassays. Instead of fluorescent or luminescent signaling molecules, primary antibodies can be tagged with DNA molecules containing orthogonal barcode sequences. This allows a combination of two or more DNA-tagged primary antibodies to be used on the same sample to detect two or more target molecules at the same time. Each individual antibody-antigen interaction can be examined via barcode readout. Considering the high diversity of DNA sequences, DNA barcoding offers much higher multiplexity far beyond the scope of conventional fluorescent or luminescent signaling molecules. Importantly, careful design of DNA barcode sequences also enables combined analysis of antibody DNA barcodes from parallel reactions using high through-put sequencing, which dramatically increases the detection throughput. However, conjugating antibodies with DNA by current methods usually results in lower signals, and a decrease in antibody affinity and specificity. Moreover, primary antibodies need to be tailor-made for each application, which is laborious and costly, and thus severely restricts the scalability of this approach. Despite the great potential, these technical challenges hamper the wide adoption of DNA barcoding in immunoassays.

Therefore, there is an unmet need to develop a system which can use DNA barcoding in immunoassays in a more effective manner.

### SUMMARY OF THE INVENTION

To overcome at least one of the above technical problems, the present disclosure provides a Multiplexed and Modular Barcoding of Antibodies (MaMBA) strategy, which is a simple and cost-effective strategy to barcode antibodies with single-stranded DNAs by using nanobodies as the adaptor. The MaMBA strategy is generally applicable to IgGs without the requirement of chemical modifications, allowing its rapid dissemination to a vast number of off-the-shelf antibodies.

According to one aspect, the present disclosure provides a composition, which comprises an antibody linked with an oligonucleotide via a disulfide bond, wherein the oligonucleotide is specific for the antibody.

According to some embodiments, the disulfide bond may be cleavable. According to some embodiments, the disulfide bond may be cut by a reducing agent. According to some embodiments, the disulfide bond may be cut by a reductive cleavage, e.g. by TCEP (Tris (2-carboxyethyl) phosphine), DTT (dithiothreitol), BME (Beta-mercaptoethanol) and the like, to remove the antibody specific oligonucleotide from the antibody.

According to some embodiments, the antibody specific oligonucleotide may comprise a hybridization chain reaction (HCR) initiator.

According to some embodiments, the antibody specific oligonucleotide may comprise a first barcode sequence which is specific for the antibody that it is associated therewith.

According to some embodiments, the barcode sequence may comprise 5 to 15 nucleotides in length. According to some specific embodiment, the barcode sequence may comprise 5 to 10 nucleotides in length, e.g. 5, 6, 7, 8, 9, or 10 nucleotides.

According to some embodiments, the oligonucleotide may further comprise a unique molecular identifier (UMI) sequence. According to some embodiments, the unique molecular identifier sequence may comprise 10 to 20 nucleotides in length. According to some embodiments, the unique molecular identifier sequence may comprise 10, 11, 12, 13, 14, 15, 16, 17, 18, 19 or 20 nucleotides.

According to some embodiments, the oligonucleotide may further comprise a second barcode sequence. The second barcode sequence may be specific for any information associated with a sample to be detected, such as the well in which the sample locates.

According to some embodiments, the oligonucleotide may further comprise an amplification region for performing a polymerase chain reaction (PCR) amplification reaction. According to some embodiments, the oligonucleotide may further comprise a pair of primer sequences for polymerase chain reaction (PCR), qPCR or sequencing.

According to some embodiments, the oligonucleotide-linked antibody may be a secondary antibody. According to some embodiments, the oligonucleotide-linked antibody may be selected from a group consisting of an anti-IgE antibody, an anti-IgM antibody, an anti-IgG antibody, an anti-IgA antibody, and an anti-IgD antibody. According to some embodiments, the oligonucleotide-linked antibody may be a nanobody. According to some embodiments, the nanobody can specifically bind to the Fc region of the anti-IgE, anti-IgM, anti-IgG, anti-IgA or anti-IgD antibody.

According some embodiments, the oligonucleotide-linked antibody may be obtained by a step of reacting an antibody with an oligonucleotide conjugated with disulfide (—S—S—). According some embodiments, the antibody may be modified with an azide moiety. According some embodiments, the oligonucleotide may be further modified with a dibenzocyclooctyne group (DBCO).

According to another aspect, the present disclosure provides a kit for detecting one or more targets, which comprises the above composition.

According to some embodiments, the kit may comprise one or more oligonucleotide-linked antibodies each comprising an antibody linked with a specific HCR initiator, and one or more amplifiers. The one or more amplifiers can trigger hybridization chain reactions. According to some embodiments, each amplifier may be conjugated with a detectable label, which may include, but do not limit to, a chemical group, such as biotin, digoxigenin, acrydite, amine, succinimidyl ester, thiol, azide, TCO, Tetrazine, Alkyne and DBCO; and a fluorophore such as FITC, Cyanine dyes, Dylight fluors, Atto dyes, Janelia Fluor dyes, Alexa Fluor dyes (e.g., Alexa Fluro 546, Alexa Fluor 488, and Alexa Fluor 647).

According to some embodiments, the kit may comprise one or more pairs of primers for specifically amplify the antibody specific oligonucleotide.

According to some embodiments, the kit may comprise one or more oligonucleotide-linked antibodies each comprising an antibody linked with an oligonucleotide, wherein the oligonucleotide comprises a barcode sequence and a region for an amplification reaction. According to some embodiments, the kit may comprise one or more pair primes for performing the amplification reaction.

According to yet another aspect, the present disclosure provides a method for detecting one or more targets in a sample by using the above composition or the above kit.

According to some embodiments, the method may comprise a step of a) incubating a first primary antibody with the above composition, to form a complex having the first primary antibody associated with the composition.

According to some embodiments, the antibody of the composition is a secondary antibody, and the complex is formed by the interaction between the first primary antibody and the secondary antibody.

According to some embodiments, the composition may comprise a secondary antibody linked with an oligonucleotide via a disulfide bond, wherein the oligonucleotide is specific for the antibody. According to some embodiments, the disulfide bond may be cut by a reducing agent.

According to some embodiments, the method may comprise the steps of: a) a first primary antibody with the above composition, to form a complex having the first primary antibody associated with the composition; b) contacting the sample with the complex; c) making an amplification reaction to produce detectable amplified products; and optionally, d) adding a reducing agent and incubating to cut the disulfide bond, and repeating steps a) to c).

According to some embodiments, one or more primary antibodies may be incubated with one or more secondary antibodies, respectively, to form one or more complexes, each complex may comprise a primary antibody and a secondary antibody associated a specific oligonucleotide. According to some embodiments, each secondary antibody may be associated with a specific oligonucleotide which can be used as a barcode. Each of the complexes formed by the primary antibody and the secondary antibody can thus be conjugated with a specific barcode.

According to some embodiments, the specific oligonucleotide may comprise an HCR initiator. According to some embodiments, the method of the present disclosure may comprise the steps of: a) a first primary antibody with the above composition, to form a complex having the first primary antibody associated with the composition; b) contacting the sample with the complex; c) performing an HCR reaction to produce detectable amplified products; and optionally, d) adding a reducing agent and incubating to cut the disulfide bond, and repeating steps a) to c). According to some embodiments, HCR amplifiers are added in step c).

According to some embodiments, the method may be used for an in situ assay or ex vivo assay.

According to some embodiments, the specific oligonucleotide may comprise a first barcode sequence. According to some embodiments, the specific oligonucleotide may further comprise a unique molecular identifier (UMI) sequence. Both the first barcode sequence and the unique identifier sequence may be specific for the antibody that they are associated therewith. According to some embodiments, the specific oligonucleotide may further comprise a second barcode sequence. The second barcode sequence may be specific for any information associated with a sample to be detected, such as the well in which the sample locates.

According to some embodiments, the method of the present disclosure may comprise the steps of: a) a first primary antibody with the above composition, to form a complex having the first primary antibody associated with the composition; b) contacting the sample with the complex; c) adding a reducing agent and incubating to cut the disulfide bond; d) collecting the specific oligonucleotides and performing an amplification reaction to detect the one or more targets; and optionally, e) repeating steps a) to d).

According to some embodiments, the method of the present disclosure may be used for semi-quantitatively or quantitatively measuring the one or more targets in the sample.

According to some embodiments, the method of the present disclosure may be used for sequencing one or more targets.

According to some embodiments, the method may comprise, before step b), a step of incubating the sample with a support which is associated with a target-specific binding substance, to capture the target in the sample. According to some embodiments, the target-specific binding substance may be a second primary antibody. According to some embodiments, the target-specific binding substance may be an antigen, and the target to be detected may be an antibody.

According to some embodiments, the support may be selected from a group consisting of a microplate and magnetic beads.

According to some embodiments, in step a), one or more first primary antibodies may incubate with one or more compositions each linked with an oligonucleotide specific for the antibody that it is linked therewith.

According to some embodiments, the method of the present disclosure may be used for high-throughput sequencing.

According to some embodiments, a washing and/or blocking step may be required after each step.

According to some embodiments, the method may comprise a step of blocking using a protein-free blocking buffer and sheared Salmon sperm DNA (ssDNA).

According to some embodiments, the method may comprise, before step a), a step of contacting the sample with a plurality of target-specific binding substances to capture the plurality of targets in the sample.

According to some embodiments, the target-specific binding substance may be antigen and the target to be detected may be an antibody.

According to some embodiments, the target-specific binding substance may be a primary antibody and the target to be detected may be an antigen recognized by this primary antibody.

The present disclosure first combined MaMBA with a fluorescent signal amplification method (immunosignal hybridization chain reaction, isHCR) and established a highly multiplexed and sensitive immunohistochemistry for spatial protein profiling (multiplexed isHCR, misHCR). Further a cleavable MaMBA is applied to increase the multiplexity of misHCR and developed a multi-round version of misHCR (misHCRⁿ). Finally, this strategy has been demonstrated to be capable of being adopted for traditional enzyme-linked immunosorbent assay (ELISA) and developed a barcode-linked immunosorbent assay (BLISA) for multiplexed and high-throughput protein detections combined with DNA sequencing.

### DESCRIPTION OF THE FIGURES

FIG. 1: Design of MaMBA. A, DNA-barcoding modules production overview. Nanobodies (Nb) targeting the Fc region of IgGs, are conjugated with an azide (N₃) group via OaAEP1 enzymatic reaction in which the C-terminal ‘NGL’ peptide sequences are recognized, cut, and ligated to ‘GVG-K (N₃)-RG’ peptide (orange dotted line shows the cut site of the enzyme). The N₃-conjugated nanobodies are linked to DBCO-modified DNA oligonucleotide (DNA oligo) via the click reaction. The final product (Nb-DNA oligo) then is ready for barcoding. B, MaMBA workflow. Each antibody is incubated with a Nb-DNA oligo employing a unique DNA barcode. Nb-DNA oligo is bound to the Fc region of antibody to form the DNA-barcoded complex (Ab-Nb 1 to Ab-Nb 3), following purification by filtration. Then the Ab-Nb-DNA oligos are available to be pooled and applied for assays (IHC, immunoassay, etc.).

FIG. 2: MaMBA applied for multiplexed in situ protein imaging by misHCR. A, Schematic of misHCR based on the HCR reaction processed by a single-strand DNA (HCR initiator) and a pair of DNA hairpins (HCR amplifiers). One-step staining is achieved by the Nb-HCR initiator-bound antibody (Ab-HCR initiator). Arrows indicate the 5′-to-3′ direction. HCR amplifiers are modified with fluorophores. B-D, Fluorescent images of mouse brain sections with or without HCR amplification. Sections are stained by anti-NeuN antibody with Cy5 dye-conjugated Nb (Nb-Cy5) or misHCR using Cy5 dye-conjugated HCR amplifiers (misHCR-Cy5), and stained by anti-Th antibody with Nb-Cy3 or misHCR-Cy3. Mean fluorescence intensity was measured for NeuN (C) and Th (D), respectively (n=3 brain sections for each group, mean±s.e.m. ****P<0.0001, ***P<0.001; two-sided t-test). Scale bar, 100 μm. E, Multiplexed misHCR imaging workflow. Antibodies barcoded with unique HCR initiators (Ab-i1 to Ab-in) are pooled and applied simultaneously to detect the antigens (Ag. 1 to Ag. n), followed by sequential rounds of imaging via hybridization and dehybridization of orthogonal HCR amplifiers (Amp.). F, Fluorescent images of human psoriatic skin section stained for DAPI and six other epitopes with three rounds of HCR imaging. Scale bar, 300 μm. Host species of antibodies are shown in the parentheses. Rb, rabbit; Mus, mouse; Th, tyrosine hydroxylase; PDGFRα, platelet-derived growth factor receptor A; KRT14, keratin 14; DCT, dopachrome tautomerase; αSMA, alpha smooth muscle actin.

FIG. 3: Cleavable MaMBA enables increased multiplexity for in situ protein imaging with misHCRⁿ. A, Cleavable DNA-barcoding module production overview. DBCO-modified DNA oligo bearing a disulfide bond is linked to N₃-conjugated Nb via click reaction. The product (Nb-SS-DNA oligo) is ready for barcoding and the DNA oligo could be removed by reductive cleavage (TCEP treatment, etc.). TCEP, Tris (2-carboxyethyl) phosphine. B, misHCRⁿ staining and imaging outline. In the first round of staining (1^{st} round of misHCR), antibodies barcoded with a unique and cleavable HCR initiator (Ab-SS-i1 to Ab-SS-in) are pooled and applied simultaneously, followed by sequential rounds of HCR imaging (image rounds) as explicated in FIG. 2E. After finishing the image rounds, HCR initiators are removed by TCEP via breaking the disulfide bonds, followed by the second round of staining (2^{nd} round of misHCR). Antibodies used in the 2^{nd} round of misHCR could be equipped with the same set of HCR initiators as in the 1^{st} round of misHCR (Ab-SS-i1′ to Ab-SS-in′) and thus for the same set of HCR amplifiers in image rounds. Then after removing HCR initiators, another round of misHCR is available to be performed, and so on. C, Fluorescent images of the dorsal raphe nucleus (DRN) in mouse brain sections stained for DAPI and 12 other epitopes by two rounds of misHCR with seven image rounds in total. The imaging location was marked in the boxed region (red) of the mouse brain atlas shown in the bottom left. Host species of antibodies are shown in the parentheses. Scale bar, 100 μm. nNOS, neuronal nitric oxide synthase; GFAP, glial fibrillary acidic protein; Th, tyrosine hydroxylase; NF-H, neurofilament-H; Tph2, tryptophan hydroxylase 2; GABA, gamma-aminobutyric acid; TMEM119, transmembrane protein 119; DDC, dopa decarboxylase; 5-HT, 5-hydroxytryptamine; Iba1, ionized calcium binding adaptor molecule. D, Zoomed-in views of the dotted-box region marked in c. Arrows indicate three types of cells (1, DDC⁺Th⁺Tph2⁻5-HT⁻ cells; 2, DDC⁺Th⁻Tph2⁺5-HT⁺ cells; 3, DDC⁺Th⁻Tph2⁻5-HT⁻ cells). Scale bar, 50 μm.

FIG. 4: Cleavable MaMBA applied for multiplexed quantitative protein detection by BLISA. A,BLISA of direct antigen detection workflow. The biomolecules of samples are directly immobilized to the surface of polystyrene microplate wells, and the target antigens (Ag. 1 to Ag. 3) are detected by the pool of unique-and-cleavable DNA-barcoded antibodies (Ab-SS-DNA barcodes). DNA barcodes are retrieved by TCEP via breaking the disulfide bonds. Released DNA barcodes are quantified by qPCR to reveal the antigen levels. B, Schematic of multiplexed detection for GFP, mCherry and α-tubulin by BLISA. Purified GFP and mCherry are loaded against each other with 1 μg/mL cell lysates (containing α-tubulin). C, qPCR results of BLISA for the three target levels in samples described in FIG. 4B. Negative control (NC) represents the sample containing the cell lysates only. ΔCt=Ct (NC)−Ct (target)+1. D, The dose-response curves for the GFP (left) and mCherry (right) analyzed from FIG. 4C. n=3 experiments, mean±s.e.m. GFP, R²=0.9893; mCherry, R²=0.9972. E, Schematic of endogenous proteins detection in HEK293T cells by BLISA and Western blot. HEK293T cells were transiently co-transfected to express GFP and mCherry, which are under control of drug-inducible promoters. The expression of GFP is controlled by the Tet-ON system and induced by ATc, a tetracycline derivative, which binds to rtTA transcription factor and allows it to bind DNA at the TRE promoter to trigger gene expression. The expression of mCherry is controlled by 3×CRE promoter and induced by FSK in the presence of 0.3 mM IBMX. ATc and FSK were titrated against each other to produce a series of culture conditions. F, G, Representative results of BLISA qPCR and Western blot bands for samples described in FIG. 4E. Three targets (GFP, mCherry and α-tubulin) were detected by BLISA and Western blot. H, Correlations of relative expression levels between BLISA and Western blot for GFP (left) and mCherry (right) which are normalized to α-tubulin. Pearson's correlations were performed. r=0.9718, P<0.0001 (left); r=0.9520, P<0.0001 (right). ATc, anhydrotetracycline hydrochloride; TRE, tetracycline response element; CRE, cAMP-response element; FSK, forskolin; IBMX, 3-Isobutyl-1-methylxanthine.

FIG. 5: Sequencing-combined BLISA for anti-SARS-CoV-2 Spike RBD IgG detection in human serum. A, BLISA of sandwich-based antibody detection workflow. The purified antigen is immobilized on the microplate so that the target antibody is captured during sample loading. After washing away other unbound biomolecules, the target antibody is detected by the DNA-barcoded secondary antibody (2^{nd} Ab-SS-DNA barcode). DNA barcode is retrieved from the plate in solution containing TCEP and a known amount of DNA barcode for normalization (norm bc), following amplicon generation for sequencing. B, Schematic of ELISA and BLISA for human IgG detection. Human IgG is immobilized on the microplate and detected by anti-human IgG antibody (from Rb host species) which is DNA-barcoded for qPCR, or follows horseradish peroxidase (HRP)-conjugated anti-Rb IgG antibody for colorimetric readout of ELISA using 3,3′,5,5′-Tetramethylbenzidine (TMB) substrates. C, Sensitivity assessments of BLISA compared with ELISA by detection of human IgG. The dose-response curves indicate higher sensitivity by BLISA with dynamic ranges shown (dotted line). Dynamic ranges described here span from the LOQ to the lower limit of 95% confidence interval (dotted line). n=3 experiments each, mean±s.e.m. ELISA, R²=0.9978, dynamic range ~3.45 logs, LOD=6.1801 ng/mL; BLISA, R²=0.9940, dynamic range ~3.60 logs, LOD=0.1407 ng/ml. LOQ, limit of quantification; LOD, limit of detection. D, DNA barcode design and amplicon generation for BLISA sequencing. DNA barcode contains an Ab barcode (Ab bc) and a unique molecular identifier (UMI). Released DNA barcode is further barcoded by a three-step PCR protocol for well barcode (well bc) addition, phasing spacers (PS) addition, and Illumina sequencing adaptors tagging. E, Annotated amplicon sequence of the final product for Illumina sequencing. F, Schematic of BLISA for human anti-SARS-CoV-2 Spike RBD IgG detection. G, BLISA sequencing results for large-scale anti-SARS-CoV-2 Spike RBD IgG detection in human serum samples. The scatterplot indicates averaged anti-SARS-CoV-2 Spike RBD IgG abundance normalized by normalization barcode for 505 human serum samples (unknown) listed by sub-groups of plate (S1 to S12). Each plate includes a blank and a negative control (neg. ctrl). n=2 replicates. Negative control represents serum from individuals that were neither SARS-CoV-2 infected nor vaccinated. H, Frequency distribution of the IgG abundances. Relative frequency (percentage) was shown, and Gaussian distribution was performed. R²=0.7369. I, Decreased coefficient of variation (CV) after normalization step. The histogram (left) presents relative distribution (percentage) of CV of each duplicate from raw (gray) or normalized (red) data. The boxplot (right) of the distribution of CV between raw and normalized groups is analyzed using two-sided paired t-test. ****P<0.0001. J-L, Validation of the BLISA sequencing results by ELISA. A portion of samples (n=13) were positioned at the same well (duplicate well G9, H9) of each plate (except for plate S12). Sample s1 was in plate S1, Sample s2 was in plate S2, etc.; Sample s12 (well E1, F1) and s13 (well E2, F2) were in plate S12. Results are presented for sequencing-combined BLISA (I) and ELISA (K). The correlations between sequencing-combined BLISA and ELISA (L) are analyzed (Pearson's correlation. r=0.9743, P<0.0001). n=2 replicates each, mean±s.e.m.

FIG. 6: 2-plex sequencing-combined BLISA for hepatitis B virus (HBV) antigens detection in human serum. A, BLISA of sandwich-based antigen detection workflow. The capture antibody (Ab^{cap}) is immobilized on the microplate. Sample and the DNA-barcoded detection antibody (Ab^{Det}-SS-DNA barcode) are simultaneously loaded. The target antigen then is trapped between Ab^{Cap} and Ab^{Det}. DNA barcode is retrieved from the plate in solution containing TCEP and a known amount of normalization barcode, following amplicon generation for sequencing. B, Schematic of 2-plex BLISA for HBsAg and HBeAg detection. C-E, 2-plex antigens detection in control serum samples by BLISA. qPCR results (C) of 2-plex BLISA indicates HBsAg (blue) and HBeAg (red) levels in double-negative and double-positive serum samples (neg. 1 to neg. 3, pos. 1 to pos. 4) which had been defined by commercial ELISA kits (D, E). n=2 replicates each, mean±s.e.m. F, Summary of the averaged antigen levels detected in negative and positive groups for HBsAg and HBeAg. mean±s.e.m. ****P<0.0001, **P<0.01, two-sided t-test. g, 2-plex BLISA sequencing results for HBsAg and HBeAg detections in human serum. The scatterplot indicates normalized averaged abundances of HBsAg and HBeAg for 498 human serum samples listed by sub-groups of plate (H1 to H12). n=2 replicates. H, Frequency distribution of the antigen abundances for HBsAg (blue) and HBeAg (red). I, Correlation of the normalized abundances between duplicate for HBsAg (left) and HBeAg (right). Pearson's correlations were performed. r=0.9973, P<0.0001 (left); r=0.9770, P<0.0001 (right). J, Validation of the sequencing data by ELISA. Samples with the antigen abundance over the total average either in HBsAg or HBeAg (n=84) were verified by ELISA. The gray line on X axis represents the cut-off value defined by the commercial ELISA kits, while the blue (HBsAg) and red (HBeAg) lines represent values below the ELISA ‘positive’ points, so-called the ‘cut-off’ values of BLISA. The dotted lines represent mean±3s.d. of the negative control group which were double-negative samples having defined by ELISA. The double-positive sample (#23) was labelled.

FIG. 7: Multiplexed detection of phospho-proteins by BLISA based on magnetic beads (MB). A, Schematic of BLISA for multiplexed detection on MB. Antigens are detected by sandwich-based assay in which capture antibodies are attached to the MB, identified via different DNA barcodes (bc. 1 to bc. n). B, MB-based BLISA workflow. Capture antibodies-bound MB are mixed and aliquoted into sample wells of the deep-well plate. Samples are loaded and incubated with MB. After washing steps utilizing the magnetic isolation to remove the unbound biomolecules, the captured antigens are detected by the DNA-barcoded detection antibodies (Ab^{Det}-SS-DNA barcodes). Then, the excess Ab^{Det}-SS-DNA barcodes are washed away, and the DNA barcodes are retrieved by TCEP. C-E, Multiplexing verification of MB-based BLISA. Seven purified phospho-proteins (p-p38α, p-ERK1/2, p-JNK, p-AMPKα1, p-CREB, p-Src, and p-Akt) are loaded within 7 wells in different concentration gradient frames (C). The qPCR results of BLISA for each antigen proteins are described (D). n=3 experiments, mean±s.e.m. R² of the dose-response curves for each antigen proteins are listed in E. F, The 7-plex BLISA and the corresponding ELISA for phospho-proteins detection in cell lysates of U87 and U937 cell lines. U87 and U937 cells were sub-cultured for 24 h, and serum-starved overnight, following different culture conditions (NT, 10% FBS for 10 min, 100 nM Aniso for 1 h, and 20 mM H₂O₂ for 10 min). Then cells were lysed and measured by the 7-plex BLISA (left) and ELISA (middle). The correlations between BLISA and ELISA for each phospho-protein target are analyzed (right) (Pearson's correlation. r=0.9241, P=0.0010; r=0.9541, P=0.0002; r=9685, P<0.0001; r=7684, P=0.0259; r=0.9845, P<0.0001; r=0.9829, P<0.0001; r=0.7636, P=0.0275). n=3 replicates, mean±s.e.m. p-p38a, phospho-p38α (T180/Y182); p-ERK1/2, phospho-ERK1 (T202/Y204)/ERK2 (T185/Y187); p-JNK, phospho-JNK Pan Specific; p-AMPKa1, phospho-AMPKα1 (T183); p-CREB, phospho-CREB (S133); p-Src, phospho-Src (Y419); p-Akt, phospho-Akt (S473); NT, non-treated; FBS, fetal bovine serum; Aniso, Anisomycin.

FIG. 8 SDS-PAGE verification of Nb-DNA oligo production and the unaffected spatial resolution of misHCR imaging. A, Non-reducing SDS-PAGE gel for purified Nanobody-NGL-His₆ (Nb-NGL-His₆), the OaAEP1 reaction product (Nb-NGV-N₃), and the Nb-DNA oligos. The same gel was first stained for the DNA oligo by nucleic acid dye and then stained for protein by Coomassie brilliant blue. B, Fluorescent images of α-tubulin in HeLa cells stained by traditional method using Alexa fluor 647-conjugated secondary antibody (2^{nd} Ab) (left) and by misHCR using Alexa Fluor 647-conjugated HCR amplifiers (right). Zoomed-in views were shown as marked in the boxed region. Scale bar, 10 μm. C, Example intensity profiles illustrated in B for straight lines drawn perpendicular to the microtubule structure. D, Spatial resolutions of traditional method (2^{nd} Ab) and misHCR by calculating the full width at half maximum (FWHM) for a series of microtubule structure intensity profiles. No significant difference was observed between 2^{nd} Ab and misHCR (n=26 for 2^{nd} Ab, n=25 for misHCR, mean±s.e.m. P=0.1567, unpaired two-sided t-test). ns, not significant.

FIG. 9 Efficient signal removal after misHCR washing steps both for image round and misHCR round, and its reproducibility for several rounds of image round washing. A-B, Fluorescent images of mouse brain sections before and after HCR amplifiers or initiators removal by formamide or TCEP. Scale bar, 500 μm (A), 300 μm (B). C, Fluorescent images of dorsal raphe nucleus (DRN) in mouse brain sections for NeuN, NF-H, and Th through five rounds of HCR imaging-and-washing cycle. Scale bar, 100 μm. D, Signal-to-noise ratios (SNRs) of each round of HCR imaging for NeuN (left) and NF-H (right). n=3 brain sections for each group, mean±s.e.m. One-way ANOVA tests were performed. The images of every group were acquired using identical microscopy settings. NF-H, neurofilament-H.

FIG. 10 misHCR imaging in healthy human skin sections. Fluorescent images of healthy human skin section stained for DAPI and six other epitopes with three rounds of HCR imaging. Host species of antibodies are shown in the parentheses. Scale bar, 200 μm. PDGFRα, platelet-derived growth factor receptor A; KRT14, keratin 14; DCT, dopachrome tautomerase; «SMA, alpha smooth muscle actin.

FIG. 11 Multi-round misHCR for nine targets using three sets of HCR initiator and amplifiers repeatedly. A, Fluorescent images of mouse brain sections stained for DAPI and 9 other targets by three rounds of misHCR with one image round of each. The imaging location was marked in the boxed region of the mouse brain atlas shown in the bottom right. Scale bar, 300 μm. B, Zoomed-in views of the dotted-box region marked in A. Different target signals were not co-localized in misHCR imaging (marked with arrows). Scale bar, 25 μm. MAP2, microtubule-associated protein 2; DDC, dopa decarboxylase; nNOS, neuronal nitric oxide synthase; Iba1, ionized calcium binding adaptor molecule 1; GFAP, glial fibrillary acidic protein; Th, tyrosine hydroxylase; NF-H, neurofilament-H; Tph2, tryptophan hydroxylase 2.

FIG. 12 Co-localization of antibody staining patterns for traditional method and misHCR. Mouse brain sections were stained with antibodies against different epitopes and then incubated with equal amounts of Cy3-conjugated secondary antibodies (2^{nd} Ab-Cy3) and Nb-HCR initiators following HCR using Alexa Fluor 647-conjugated amplifiers (misHCR-647). Scale bar, 100 μm.

FIG. 13 Endogenous proteins detection in HEK293T cells by BLISA and Western blot, and sequencing-combined BLISA for multiplexed detection of GFP, mCherry and α-tubulin. A, Endogenous GFP detection. HEK293T cells were transiently transfected to express GFP, which are under control of the Tet-ON system and induced by ATc, a tetracycline derivative, which binds to rtTA transcription factor and allows it to bind DNA at the TRE promoter to trigger gene expression. B, Representative results of BLISA qPCR and Western blot bands (left panel), and their corresponding correlation of relative expression levels of GFP (normalized to α-tubulin) (right panel). Pearson's correlations were performed. r=0.9724, P<0.0001. C, Endogenous mCherry detection. HEK293T cells were transiently transfected to express mCherry, which are under control of the 3×CRE promoter and induced by FSK in the presence of 0.3 mM IBMX. D, Representative results of BLISA qPCR and Western blot bands (left panel), and their corresponding correlation of relative expression levels of mCherry (normalized to α-tubulin) (right panel). Pearson's correlations were performed. r=0.9647, P<0.0001. E, DNA barcode design and amplicon generation for BLISA sequencing. DNA barcode contains an Ab barcode (Ab bc) and a unique molecular identifier (UMI). Released DNA barcode is further barcoded by a two-step PCR protocol for well barcode (well bc) addition and illumina sequencing adaptors tagging. F, UMI counts for the three targets (GFP, mCherry, and α-tubulin) in the samples as designed in samples containing purified GFP and mCherry loaded against each other with 1 μg/mL cell lysates (α-tubulin). G, The dose-response curves analyzed from F for GFP (upper) and mCherry (bottom). R²=0.9827, 0.9843. n=3 experiments, mean±s.e.m. ATc, anhydrotetracycline hydrochloride; TRE, tetracycline response element; CRE, cAMP-response element; FSK, forskolin; IBMX, 3-Isobutyl-1-methylxanthine.

FIG. 14 Reproductivity and plate-to-plate variability of sequencing-combined BLISA. A, Correlation of the normalized abundances between duplicate for the IgG detection. Pearson's correlations were performed. r=0.9882, P<0.0001. B, Results of BLISA by qPCR for a portion of samples (n=13) which were positioned through different plates in the sequencing. n=2 replicates each, mean±s.e.m. C, Consistency between two BLISA outputs, qPCR and sequencing. The correlation between sequencing results and qPCR results of BLISA for the 13 samples are analyzed. Pearson's correlation. r=0.9354, P<0.0001).

FIG. 15 Standard curves of purified phospho-proteins in BLISA compared with ELISA. A, Standard curves of purified phospho-proteins (p-p38α, p-ERK1/2, p-JNK, p-AMPKα1, p-CREB, p-Src, and p-Akt) in BLISA and ELISA. B, R² of the standard curves.

### DETAILED DESCRIPTION OF THE INVENTION

Before the present methods and compositions are described, it is to be understood that this invention is not limited to a particular method or composition described and as such may vary. It is also to be understood that the terminology used herein is for the purpose of describing particular embodiments only, and is not intended to be limiting, since the scope of the present invention will be limited only by the appended claims.

As the modular adaptor, nanobodies, which are single-domain antibodies derived from camelid heavy-chain antibodies, increasingly become popular as powerful tools in cell biology, structural biology, and therapy, taking advantage of their excellent functionality (small size, easy production, flexible manipulation, etc). Using the secondary nanobodies (nanobodies against antibodies) with the monovalent property, different antibodies even sharing the same host species can be simply pre-incubated with the secondary nanobodies and then applied to sample together, which is infeasible for bivalent secondary antibodies. In this study, we provided MaMBA, an efficient modular barcoding strategy that is generally applicable to antibodies via nanobodies for simple and site-specific DNA barcoding. Firstly, we utilized this strategy to develop misHCR, a multiplexed in situ protein imaging method, which combined HCR technology with immunohistochemistry using orthogonal HCR initiator-barcoded antibodies. To achieve higher imaging throughput, we developed a cleavable MaMBA by employing a disulfide linker between nanobody and DNA, and further applied it to the multi-round version of misHCR (misHCRⁿ). Next, we extended the cleavable MaMBA strategy to immunoassay, and developed BLISA, a DNA barcode-driven, multiplexed quantitative protein detection method using the readout of qPCR or high-throughput DNA sequencing. To investigate the potential of BLISA in diagnostics for population-scale testing, we performed sequencing-combined BLISA for anti-SARS-CoV-2 Spike RBD IgG detection in ~500 human serum samples, and further performed 2-plex BLISA for HBV antigens (HBsAg and HBeAg) detection in ~500 human serum samples. Overall, MaMBA offers a simple and efficient strategy to barcode antibodies with DNA, with the feasibility and versatility to apply for different immunodetection assays.

Fluorescent immunostaining has become a routine approach in both biological and clinical laboratories. However, due to the spectral overlap of fluorophores, traditional methods offer limited multiplexing targets. Although strategies like sequential antibody staining (strips the bound antibodies and starts a new round of staining) have achieved enhanced multiplexing, it requires multiple rounds of antibodies incubation (hours or even days per cycle) and takes weeks to image tens of targets. By contrast, DNA barcoding approaches enable simultaneous antibodies binding with orthogonal DNA barcodes, followed by sequential docking-strand readout to achieve faster multiplexed imaging. However, owing to the overlap of antibody species, DNA conjugation should be applied to antibodies, which makes sequential docking-strand imaging weaker signal than traditional immunostaining because it loses the signal enhancement from using secondary antibodies. As a result, DNA-based signal amplification methods were applied such as rolling circle amplification (RCA), signal amplification by exchange reaction (SEBAR), and HCR. HCR utilizes a pair of HCR hairpins that iteratively opened, triggered by a complementary HCR initiator. Signal amplification by HCR happens in situ and could be tuned by changing the concentration of HCR amplifiers. Thus, misHCR not only achieved the multiplexing relied on the power of DNA barcodes, but also obtained amplified signal levels via the HCR reaction. What's more, the potential of misHCR is not limited to fluorescent imaging. Recently, methods based on Raman microscopy were developed for multiplexed protein imaging, which utilize orthogonal Raman dyes that possess much narrower vibrational peaks than fluorescence. Raman dye imaging shows the advantage of multiplexing in one shot, which could greatly accelerate the experimental procedure.

Immunoassays based on DNA-barcoded antibodies have shown the advantage that dramatically enhances the sensitivity of conventional immunoassays. For example, taking advantage of DNA amplification techniques, immuno-PCR and its related methods showed excellent performance in ultrasensitive detection for low abundance proteins, typically leading to 10-10, 000-fold increase in sensitivity compared with analogous enzyme-amplified immunoassay. Therefore, combined with the sequencing technique, a variety of immunoassays utilizing DNA-barcoded antibodies were developed and demonstrated for highly multiplexed and high-throughput proteins detection (e.g. ID-seq), further single-cell level readout (e.g. CITE-seq), and even transcriptome-combined expression profiling (e.g. SCITO-seq). However, the antibody-DNA oligo conjugates were mostly generated by one-to-one biochemical conjugation methods, which means every single combination of antibody and DNA barcode should go through a conjugation process (activation, conjugation, purification, and examination), revealing labor-intensive and costly procedure. Additionally, although there are a vast number of commercial off-the-shelf antibodies, the different vendor usually provides different buffer recipe for antibody preservation (salts, sodium azide, glycerol, etc), which may impact the chemical reactions and introduce incoordinate conjugates ratios for different antibodies. To avoid that, antibodies should be buffer exchanged before conjugation, which further increases the workload. Thus, using one-to-one biochemical conjugation strategy is not friendly for large-scale antibodies production, and obstructs the widespread applications. To overcome these problems, BLISA adopts the MaMBA strategy for non-covalent and site-specific barcoding of antibodies using secondary nanobodies as modular mediators. Pipeline-synthesized nanobody-DNA conjugates can be directly mixed with antibodies, and each purified DNA-barcoded antibody then is ready for the multiplexed assembly. This straightforward DNA barcoding workflow is designed to be suitable for large-scale pipeline production, which enables wider applications of DNA-barcoded-antibodies-based techniques with higher efficiency and lower costs.

Unless defined otherwise, all technical and scientific terms used herein have the same meaning as commonly understood by those skilled in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

### Definitions

Unless otherwise defined, all scientific and technical terms used herein have the same meaning as commonly understood by those skilled in the art to which this technology belongs.

It is noted that as used herein and in the appended claims, the singular forms “a,” “an,” and “the” include plural referents unless the context clearly dictates otherwise. Thus, for example, reference to “a recombinant AAV virion” includes a plurality of such virions and reference to “microglia” includes reference to one or more microglia cells and equivalents thereof known to those skilled in the art, and so forth.

The term “hybridization chain reaction” or “HCR” used herein is a technique, based on a chain reaction of recognition and hybridization events between two sets of DNA hairpin molecules, offers an enzyme-free alternative for the rapid detection of specific DNA sequences. HCR uses a pair of complementary, kinetically trapped hairpin oligomers to propagate a chain reaction of hybridization events.

The term “hybridization chain reaction initiator” or “HCR initiator” used herein refers to a nucleic acid region that can trigger the polymerization of two metastable HCR hairpin monomer species to form an HCR amplification polymer. An exposed HCR initiator is functional and triggers polymerization of the metastable HCR hairpin monomers under polymerizing conditions. A sequestered HCR initiator is non-functional and does not trigger polymerization of the metastable HCR hairpins monomers under polymerizing conditions. A sequestered HCR initiator (hence, initially non-functional) can be exposed (hence, becoming functional) upon binding of another molecule to the sequestering molecule.

The term “nanobody” as defined herein includes, but not limited to a VHH sequence. In some embodiments, the nanobody of the present disclosure specifically binds to Fc domain of the primary antibody.

The term “unique molecular identifier” or “UMI” as used herein comprises a random oligonucleotide sequence that is increasingly used in high-throughput sequencing.

The following examples are set forth to provide the ordinarily skilled artisan with a complete disclosure and description for guidance as to how to make and use the variant AAV capsids disclosed herein, and are not intended to limit the scope of the invention disclosed herein. In addition, the following examples are not intended to represent that the experiments below are all or the only experiments.

### Methods

### Reagents and Reagent Preparation.

DNA oligos were synthesized by Thermo Fisher Scientific, Genewiz and Sangon Biotech. Detailed sequences and modifications of DNA oligos are listed in Table 1. All oligos were dissolved in nuclease-free water (Thermo Fisher Scientific, AM9932) and stored at −20° C.

Sodium tetraborate decahydrate (NaB; S9640), Triton X-100 (T8787), dextran sulfate (D8906), Tris (2-carboxyethyl)phosphine (TCEP; 646547), Tween-20 (P9416), protease inhibitor cocktail (P8340), and 3,3′,5,5′-Tetramethylbenzidine (TMB; T0440) were purchased from Sigma-Aldrich. 20× sodium chloride citrate (SSC) buffer (AM9763), sheared Salmon sperm DNA (AM9680), cell extraction buffer (FNN0011), and protein-free blocking buffer (PFBB; 37572) were purchased from Thermo fisher Scientific.

TABLE 1
Detailed sequences and modifications of DNA oligos.

| Name | Sequence (5′ to 3′) | Modifications |
|---|---|---|
| B1 I1 | ATATAGCATTCTTTCTTGAGGAGGGCAGCAAACGGGAAGAG | 5′ DBCO or 5′ Amino C6 |
| | (SEQ ID NO: 1) | |
| B2 I1 | ATATAAGCTCAGTCCATCCTCGTAAATCCTCATCAATCATC | 5′ DBCO or 5′ Amino C6 |
| | (SEQ ID NO: 2) | |
| B3 I1 | ATATAAAAGTCTAATCCGTCCCTGCCTCTATATCTCCACTC | 5′ DBCO or 5′ Amino C6 |
| | (SEQ ID NO: 3) | |
| B4 I1 | ATATACACATTTACAGACCTCAACCTACCTCCAACTCTCAC | 5′ DBCO or 5′ Amino C6 |
| | (SEQ ID NO: 4) | |
| B5 I1 | ATATACACTTCATATCACTCACTCCCAATCTCTATCTACCC | 5′ DBCO or 5′ Amino C6 |
| | (SEQ ID NO: 5) | |
| B8 I1 | ATATACCTTGATTATCTCGTCTCCATCACTCGCACTCTACC | 5′ DBCO or 5′ Amino C6 |
| | (SEQ ID NO: 6) | |
| B9 I1 | ATATACACGTATCTACTCCACTCTCAGCACACTCCCAACCC | 5′ DBCO or 5′ Amino C6 |
| | (SEQ ID NO: 7) | |
| B10 I1 | ATATACCTCAAGATACTCCTCTACCTACTCGACTACCCTAG | 5′ DBCO or 5′ Amino C6 |
| | (SEQ ID NO: 8) | |
| B11 I1 | ATATACGCTTAGATATCACTCCTACGTCGACCACACTCATC | 5′ DBCO or 5′ Amino C6 |
| | (SEQ ID NO: 9) | |
| B13 I1 | ATATAAGGTAACGCCTTCCTGCTTTATGCTCAACATACAAC | 5′ DBCO or 5′ Amino C6 |
| | (SEQ ID NO: 10) | |
| B14 I1 | ATATAAATGTCAATAGCGAGCGACCCTATATTTCTGCACAG | 5′ DBCO or 5′ Amino C6 |
| | (SEQ ID NO: 11) | |
| B15 I1 | ATATACAGATTAACACACCACAAGGTATCTCGAACACTCTC | 5′ DBCO or 5′ Amino C6 |
| | (SEQ ID NO: 12) | |
| B17 I1 | ATATACGATTGTTTGTTGTGGACGCATGCTAATCGGATGAG | 5′ DBCO or 5′ Amino C6 |
| | (SEQ ID NO: 13) | |
| B1 Amplifier H1 | CGTAAAGGAAGACTCTTCCCGTTTGCTGCCCTCCTCGCATT | 5′ Alexa Fluor 546 or 5′ Cy3 |
| | CTTTCTTGAGGAGGGCAGCAAACGGGAAGAG | |
| | (SEQ ID NO: 14) | |
| B1 Amplifier H2 | GAGGAGGGCAGCAAACGGGAAGAGTCTTCCTTTACGCTCTT | 3′ Alexa Fluor 546 or 3′ Cy3 |
| | CCCGTTTGCTGCCCTCCTCAAGAAAGAATGC | |
| | (SEQ ID NO: 15) | |
| B2 Amplifier H1 | GGCGGTTTACTGGATGATTGATGAGGATTTACGAGGAGCTC | 5′ Alexa Fluor 594 |
| | AGTCCATCCTCGTAAATCCTCATCAATCATC | |
| | (SEQ ID NO: 16) | |
| B2 Amplifier H2 | CCTCGTAAATCCTCATCAATCATCCAGTAAACCGCCGATGA | 3′ Alexa Fluor 594 |
| | TTGATGAGGATTTACGAGGATGGACTGAGCT | |
| | (SEQ ID NO: 17) | |
| B3 Amplifier H1 | CGGGTTAAAGTTGAGTGGAGATATAGAGGCAGGGACAAAGT | 5′ Alexa Fluor 488 |
| | CTAATCCGTCCCTGCCTCTATATCTCCACTC | |
| | (SEQ ID NO: 18) | |
| B3 Amplifier H2 | GTCCCTGCCTCTATATCTCCACTCAACTTTAACCCGGAGTG | 3′ Alexa Fluor 488 |
| | GAGATATAGAGGCAGGGACGGATTAGACTTT | |
| | (SEQ ID NO: 19) | |
| B4 Amplifier H1 | GAAGCGAATATGGTGAGAGTTGGAGGTAGGTTGAGGCACAT | 5′ Alexa Fluor 647 or 5′ Cy5 |
| | TTACAGACCTCAACCTACCTCCAACTCTCAC | |
| | (SEQ ID NO: 20) | |
| B4 Amplifier H2 | CCTCAACCTACCTCCAACTCTCACCATATTCGCTTCGTGAG | 3′ Alexa Fluor 647 or 3′ Cy5 |
| | AGTTGGAGGTAGGTTGAGGTCTGTAAATGTG | |
| | (SEQ ID NO: 21) | |
| B5 Amplifier H1 | ATTGGATTTGTAGGGTAGATAGAGATTGGGAGTGAGCACTT | 5′ Alexa Fluor 488 |
| | CCATATCACTCACTCCAATCTCTATCTACCC | |
| | (SEQ ID NO: 22) | |
| B5 Amplifier H2 | CTCACTCCCAATCTCTATCTACCCTACAAATCCAATGGGTA | 3′ Alexa Fluor 488 |
| | GATAGAGATTGGGAGTGAGTGATATGAAGTG | |
| | (SEQ ID NO: 23) | |
| B8 Amplifier H1 | CGTCTCCATCACTCGCACTCTACCGTCAAGTCAAACGGTAG | 5′ Alexa Fluor 546 |
| | AGTGCGAGTGATGGAGACGAGATAATCAAGG | |
| | (SEQ ID NO: 24) | |
| B8 Amplifier H2 | GTTTGACTTGACGGTAGAGTGCGAGTGATGGAGACGCCTTG | 3′ Alexa Fluor 546 |
| | ATTATCTCGTCTCCATCACTCGCACTCTACC | |
| | (SEQ ID NO: 25) | |
| B9 Amplifier H1 | CCACTCTCAGCACACTCCCAACCCTACTACAAGCTCGGGTT | 5′ Alexa Fluor 647 |
| | GGGAGTGTGCTGAGAGTGGAGTAGATACGTG | |
| | (SEQ ID NO: 26) | |
| B9 Amplifier H2 | GAGCTTGTAGTAGGGTTGGGAGTGTGCTGAGAGTGGCACGT | 3′ Alexa Fluor 647 |
| | ATCTACTCCACTCTCAGCACACTCCCAACCC | |
| | (SEQ ID NO: 27) | |
| B10 Amplifier H1 | CCTCTACCTACTCGACTACCCTAGCCGTAACTTCACCTAGG | 5′ Alexa Fluor 488 |
| | GTAGTCGAGTAGGTAGAGGAGTATCTTGAGG | |
| | (SEQ ID NO: 28) | |
| B10 Amplifier H2 | GTGAAGTTACGGCTAGGGTAGTCGAGTAGGTAGAGGCCTCA | 3′ Alexa Fluor 488 |
| | AGATACTCCTCTACCTACTCGACTACCCTAG | |
| | (SEQ ID NO: 29) | |
| B11 Amplifier H1 | ACTCCTACGTCGACCACACTCATCCTGCATGTTCCCGATGA | 5′ Alexa Fluor $46 |
| | GTGTGGTCGACGTAGGAGTGATATCTAAGCG | |
| | (SEQ ID NO: 30) | |
| B11 Amplifier H2 | GGGAACATGCAGGATGAGTGTGGTCGACGTAGGAGTCGCTT | 3′ Alexa Fluor 546 |
| | AGATATCACTCCTACGTCGACCACACTCATC | |
| | (SEQ ID NO: 31) | |
| B13 Amplifier H1 | CCTGCTTTATGCTCAACATACAACCAGAAATGCGGCGTTGT | 5′ Alexa Fluor 647 |
| | ATGTTGAGCATAAAGCAGGAAGGCGTTACCT | |
| | (SEQ ID NO: 32) | |
| B13 Amplifier H2 | GCCGCATTTCTGGTTGTATGTTGAGCATAAAGCAGGAGGTA | 3′ Alexa Fluor 647 |
| | ACGCCTTCCTGCTTTATGCTCAACATACAAC | |
| | (SEQ ID NO: 33) | |
| B14 Amplifier H1 | GAGCGACCCTATATTTCTGCACAGAAGTTATACCGGCTGTG | 5′ Alexa Fluor 488 |
| | GAGAAATATAGGGTCGCTCGCTATTGACATT | |
| | (SEQ ID NO: 34) | |
| B14 Amplifier H2 | CCGGTATAACTTCTGTGCAGAAATATAGGGTCGCTCAATGT | 3′ Alexa Fluor 488 |
| | CAATAGCGAGCGACCCTATATTTCTGCACAG | |
| | (SEQ ID NO: 35) | |
| B15 Amplifier H1 | CCACAAGGTATCTCGAACACTCTCCAAATTGGCTACGAGAG | 5′ Alexa Fluor 546 |
| | TGTTCGAGATACCTTGTGGTGTGTTAATCTG | |
| | (SEQ ID NO: 36) | |
| B15 Amplifier H2 | GTAGCCAATTTGGAGAGTGTTCGAGATACCTTGTGGCAGAT | 3′ Alexa Fluor 546 |
| | TAACACACCACAAGGTATCTCGAACACTCTC | |
| | (SEQ ID NO: 37) | |
| B17 Amplifier H1 | GTGGACACCTGCTAATCGGATGAGTGTTCGTTATCGCTCAT | 5′ Alexa Fluor 647 |
| | CCGATTAGCAGGTGTCCACAACAAACAATCG | |
| | (SEQ ID NO: 38) | |
| B17 Amplifier H2 | CGATAACGAACACTCATCCGATTAGCAGGTGTCCACCGATT | 3′ Alexa Fluor 647 |
| | GTTTGTTGTGGACACCTGCTAATCGGATGAG | |
| | (SEQ ID NO: 39) | |
| qbc. 1 (for qPCR) | TCTTGTGGAAAGGACGAAACACGTGATNNNNNNNNNNNNNN | 5′ Amino C6 |
| | NGTCTGGAGCATGCGCTTTAG | |
| | (SEQ ID NO: 40) | |
| qbc. 2 for qPCR | TACACGACGCTCTTCCGATCTCGTGATNNNNNNNNNNNNNN | 5′ Amino C6 |
| | NTTGAAAAAGTGGCACCGAGT | |
| | (SEQ ID NO: 41) | |
| qbc. 3 (for qPCR) | ACACGTCTGAACTCCAGTCACCGTGATNNNNNNNNNNNNNN | 5′ Amino C6 |
| | NCGTATGCCGTCTTCTGCTTG | |
| | (SEQ ID NO: 42) | |
| qbc. 4 (for qPCR) | GACAGTTCGAGTTTGAAGCGCGTGATNNNNNNNNNNNNNNN | 5′ Amino C6 |
| | CTAGACGTGGGAGTGCATACT | |
| | (SEQ ID NO: 43) | |
| qbc. 5 (for qPCR) | GAAAGATCTGGCTGCCATGCCGTGATNNNNNNNNNNNNNNN | 5′ Amino C6 |
| | TCGCAAACCTGGTTGGAATCA | |
| | (SEQ ID NO: 44) | |
| qbc. 6 (for qPCR) | AGATGACGTCGATTGTTGGTCGTGATNNNNNNNNNNNNNNN | 5′ Amino C6 |
| | CATGGAGGTTGTGTCACCGTA | |
| | (SEQ ID NO: 45) | |
| qbc. 7 (for qPCR) | TCAGGTGCATAGGAGTCAGCCGTGATNNNNNNNNNNNNNNN | 5′ Amino C6 |
| | ATGCTGTCAGTTCATGGCTCC | |
| | (SEQ ID NO: 46) | |
| qbc. 8 (for qPCR) | AACGTTAGTGGACAGAGGCTCGTGATNNNNNNNNNNNNNNN | 5′ Amino C6 |
| | GGTAGAGTTCAAGGACGCACA | |
| | (SEQ ID NO: 47) | |
| sbc. 1 (for sequencing) | TCTTGTGGAAAGGACGAAACACGTGATNNNNNNNNNNNNNN | 5′ Amino C6 |
| | NGTCTGGAGCATGCGCTTTAG | |
| | (SEQ ID NO: 48) | |
| sbc. 2 (for sequencing) | TCTTGTGGAAAGGACGAAACAATCACGNNNNNNNNNNNNNN | 5′ Amino C6 |
| | NGTCTGGAGCATGCGCTTTAG | |
| | (SEQ ID NO: 49) | |
| sbc. 3 (for sequencing) | TCTTGTGGAAAGGACGAAACACGATGTNNNNNNNNNNNNNN | 5′ Amino C6 |
| | NGTCTGGAGCATGCGCTTTAG | |
| | (SEQ ID NO: 50) | |
| sbc. 4 (for sequencing) | TCTTGTGGAAAGGACGAAACATTAGGCNNNNNNNNNNNNNN | 5′ Amino C6 |
| | NGTCTGGAGCATGCGCTTTAG | |
| | (SEQ ID NO: 51) | |
| sbc. 5 (for sequencing) | TCTTGTGGAAAGGACGAAACATGACCANNNNNNNNNNNNNN | 5′ Amino C6 |
| | NNGTCTGGAGCATGCGCTTTAG | |
| | (SEQ ID NO: 52) | |
| shc. 6 (for sequencing) | TCTTGTGGAAAGGACGAAACAACAGTGNNNNNNNNNNNNNN | 5′ Amino C6 |
| | NGTCTGGAGCATGCGCTTTAG | |
| | (SEQ ID NO: 53) | |
| sbc. 7 (for sequencing) | TCTTGTGGAAAGGACGAAACAGCCAATNNNNNNNNNNNNNN | 5′ Amino C6 |
| | NGTCTGGAGCATGCGCTTTAG | |
| | (SEQ ID NO: 54) | |
| shc. 8 (for sequencing) | TCTTGTGGAAAGGACGAAACACAGATCNNNNNNNNNNNNNN | 5′ Amino C6 |
| | NGTCTGGAGCATGCGCTTTAG | |
| | (SEQ ID NO: 55) | |
| Norm bc. 1 (for | TCTTGTGGAAAGGACGAAACAACAGTGNNNNNNNNNNNNNN | |
| sequencing) | NGTCTGGAGCATGCGCTTTAG | |
| | (SEQ ID NO: 56) | |
| Norm bc. 2 (for | TCTTGTGGAAAGGACGAAACAGCCAATNNNNNNNNNNNNNN | |
| sequencing) | NGTCTGGAGCATGCGCTTTAG | |
| | (SEQ ID NO: 57) | |
| Norm bc. 3 (for | TCTTGTGGAAAGGACGAAACACAGATCNNNNNNNNNNNNNN | |
| sequencing) | NGTCTGGAGCATGCGCTTTAG | |
| | (SEQ ID NO: 58) | |
| Norm bc. 4 (for | TCTTGTGGAAAGGACGAAACAACTTGANNNNNNNNNNNNNN | |
| sequencing) | NGTCTGGAGCATGCGCTTTAG | |
| | (SEQ ID NO: 59) | |
| Norm bc. 5 (for | TCTTGTGGAAAGGACGAAACAGATCAGNNNNNNNNNNNNNN | |
| sequencing) | NGTCTGGAGCATGCGCTTTAG | |
| | (SEQ ID NO: 60) | |

The sequences of antibody-specific barcodes were underlined.

### Plasmid Construction.

DNA fragments were amplified by PCR using primers (Genewiz) with 17-20 bp overlaps for Gibson assembly, and plasmid sequences were verified using Sanger sequencing. For protein purification, pET28a-His₆-Ubiquitin-OaAEP1 (C247A) was constructed as described¹, and DNA sequences encoding TP897-NGL-His₆ and TP1107-NGL-His₆ were synthesized (Genewiz) and cloned into pET28a vector. EGFP and mCherry sequences were cloned into the pET28a vector fused with C-terminal His₆-tag sequences. To assess BLISA multiplexity for endogenous protein detection, EGFP and rtTA sequences were cloned into a pLJM1 vector (Addgene, 91980)² under control of a TRE promoter and the PGK promoter (by replacing the original puromycin N-acetyltransferase gene) respectively, and sequences encoding mCherry were cloned into another pLJM1 vector following a 3×CRE promoter.

### Protein Expression and Purification.

Recombinant OaAEP1 (C247A) was expressed and obtained. Briefly, pET28a-His₆-Ubiquitin-OaAEP1 (C247A) was transformed into E. coli SHuffle and the protein expression was induced overnight with 1 mM IPTG at 18° C. Cells were harvested by centrifugation, resuspended in lysis buffer (50 mM NaH₂PO₄, pH 8.0, 300 mM NaCl, 10 mM imidazole), and lysed by sonication. Cell debris was cleared via 1 hr of centrifugation at 39, 000 g at 4° C. The supernatant was bound to Ni-NTA resin, washed with AEP wash buffer (50 mM NaH₂PO₄, pH 8.0, 0.3 M NaCl, 20 mM imidazole), and eluted with elution buffer (50 mM NaH₂PO₄, pH 8.0, 0.3 M NaCl, 250 mM imidazole). The protein was further purified through size exclusion column (Superdex 75 increase 10/300 GL) pre-equilibrated in PBS buffer. To self-activate OaAEP1, fractions containing the protein were pooled and incubated overnight at room temperature (RT), with 1 mM EDTA and 0.5 mM Tris (2-carboxyethyl) phosphine hydrochloride added to the solution and the pH adjusted to 4.0 by titrating glacial acetic acid. After filtered by 0.22 μm membrane and 1:8 diluted in 50 mM Acetate at pH 4.0, the mature protein was purified further by cation exchange (HiTrap SP column) and eluted with a salt gradient (0-100% Storage buffer (50 mM sodium acetate, pH 4.0, 10% Glycerol, 1 M NaCl); 10 column volumes). Fractions containing mature AEP were pooled and concentrated using 10 K MWCO concentrator (Sartorius). The final product was analyzed by SDS-PAGE, quantified via A₂₈₀ reading (NanoPhotometer® N60, IMPLEN) according to the protein molecular weight and extinctions coefficient, and then stored in aliquots at −80° C. until use.

Nanobodies against Fc domain of IgG (TP897 and TP1107) were expressed and purified. Briefly, pET21a-TP897-NGL-His₆ and pET21a-TP1107-NGL-His₆ were transformed into E. coli Shuffle. Expression was induced for 16-18 h with 1 mM IPTG at 28° C. After harvested by centrifugation, cells were resuspended in Nb lysis buffer (50 mM HEPES, pH 7.5, 300 mM NaCl, 5 mM Imidazole, 10% Glycerol) and lysed by sonication. Cells debris was removed by centrifugation and the supernatant was applied to Co-NTA resin. The resin was washed with Nb wash buffer (20 mM HEPES, pH 7.5, 300 mM NaCl. 10 mM Imidazole, 10% Glycerol) and the His-tagged nanobodies were eluted with Nb elution buffer (20 mM HEPES, pH 7.5, 300 mM NaCl, 500 mM Imidazole, 10% Glycerol). The nanobodies were further purified and buffer exchanged by size exclusion column (SEC) in Nb exchange buffer (20 mM HEPES, pH 7.5, 300 mM NaCl, 10% Glycerol). Fractions containing nanobodies were pooled and concentrated using Amicon 3 K MWCO concentrator (Millipore). Protein concentrations were determined by A₂₈₀ reading as described above. Nanobodies were stored in aliquots at −80° C. until use. The amino acids sequences of TP897-NGL-His₆ and TP1107-NGL-His₆ are listed in Table 2.

pET28a-GFP and pET28a-mCherry were transformed into E. coli BL21 (DE3). Expression was induced for 16-18 h with 0.5 mM IPTG at 16° C. GFP and mCherry were purified using Co-NTA resin as described above, and were further purified by SEC in PBS. Proteins were concentrated using Amicon 10 K MWCO concentrator and quantified by A₂₈₀ reading.

TABLE 2
Amino acids sequences of nanobodies.

| Nanobodies | Sequences |
|---|---|
| TP897- | MQVQLVECGGGLVQAGDSLRLSCVASGRSLDGATM |
| NGL-His₆ | RWYRQAPGKEREFVAGIFWDEIGTEYADTAKGRFT |
| | ISRDNAKNTIYLQMTNLRSEDTAMYYCNGLVEGGE |
| | YWGKGTLVTVSSNGLHHHHHH |
| | (SEQ ID NO: 61) |
| TP1107- | MQVQLVECGGGLVQPGGSLRLSCAASGFTFSDTWM |
| NGL-His₆ | NWVRQAPGKGLYWISAINPDGGNTAYADSVKGRFT |
| | ISRDNAKNMVYLQMDNLRPEDTAMYYCAKGWVRLP |
| | DPDLVRGQGTQVTVSSNGLHHHHHH |
| | (SEQ ID NO: 62) |

### C-Terminal Labeling of Nanobodies by OaAEP1 Reaction.

Purified NGL-tagged nanobodies were labeled C-terminally using GVG-K (N₃)-RG by OaAEP1 reaction. This was carried out by reacting 50 μM nanobody, 1 mM GVG-K (N₃)-RG, and 750 nM OaAEP1 (C247A) in reaction buffer (100 mM sodium phosphate buffer, pH 6.5, supplemented with 2 mM DTT) overnight with gentle shaking at RT. The reaction mixture was then 1:1 diluted in Nb lysis buffer and bound to Ni-NTA resin for removing GL-His₆ and unreacted His-tagged nanobody. Resin was washed with Nb lysis buffer until A₂₈₀ reached baseline. Flowthrough and wash fractions containing Azide-labeled nanobodies were pooled and remaining GVG-K (N₃)-RG was removed using 3,000 MWCO concentrator. Azide-labeled nanobodies were also quantified by A₂₈₀ reading and analyzed by SDS-PAGE.

### Nanobody-Fluorophore Conjugation.

40 μM azide-labeled nanobodies was mixed with 10-fold molar excess of DBCO-Cy3 (777366, Sigma-Aldrich) or DBCO-Cy5 (777374, Sigma-Aldrich) and reacted at RT with shaking for 4 hr. Excess fluorophores were removed by Zeba spin desalting column (7k MWCO) twice. Protein concentration and the degree of labeling (DOL) were measured by NanoPhotometer® N60 with dye correction. Fluorophore-conjugated nanobody was analyzed by SDS-PAGE.

### Nanobody-DNA Oligonucleotide Conjugation.

For Nb-SS-HCR initiator and Nb-SS-DNA barcode, we used DBCO-PEG₃-SS-NHS (10 mM in anhydrous DMSO; CP-2089, Conju-Probe, LLC) as the linker. HCR initiators and DNA barcodes modified with a 5′ NH₂-C6 modification were reacted with DBCO-PEG₃-SS-NHS (20-fold molar excess) in 0.091 M NaB (pH 8.5) at RT for 2 h with gentle shaking. To remove excess linker, oligonucleotides were precipitated by mixing with one-tenth volume of 3 M sodium acetate at pH 5.2 and 2 volumes of cold absolute ethanol following incubation for 1 h at −20° C. After a 30-min spin at 20, 000 g at 4° C., supernatant was removed and pellet was carefully rinsed twice by cold 70% ethanol. Pellet was air-dried and re-dissolved in TE buffer. Excess cross-linkers were further removed by Zeba spin desalting column (7K MWCO).

HCR initiators modified with a 5′ DBCO moiety (2-fold molar excess) or the DBCO-PEG₃-SS-conjugated oligos (2-fold molar excess) were mixed with 25 μM azide-labeled nanobodies, and reacted at 4° C. with shaking for 16 h. The DNA oligo-conjugated nanobodies were analyzed by SDS-PAGE.

### DNA Barcoding to Antibodies by MaMBA Strategy.

Antibodies were premixed with 3-fold molar excess of DNA oligo-conjugated nanobodies (Nb-HCR initiator, Nb-SS-HCR initiator, or Nb-SS-DNA barcode) respectively in 50 μL premixing buffer (0.1% BSA, 0.05% Triton X-100, 5 mM EDTA in PBS) at RT with shaking for 3-5 h. The mixtures were then added to Amicon Ultra-0.5 100 K centrifugal filters and washed ten times with 400 μL PBS containing 0.05% Tween-20 and 5 mM EDTA. Then the purified DNA-barcoded antibodies were stored at 4° C. with 0.03% NaN₃ or stored at −20° C. with 50% glycerol until assaying.

### Mice.

Animal care and use followed the approval of the Animal Care and Use Committee of the National Institute of Biological Sciences, Beijing (Approval ID: NIBSLuoM15C), in accordance with the Regulations for the Administration of Affairs Concerning Experimental Animals of China. C57BL/6N mice were purchased from Beijing Vital River Laboratory Animal Technology Co., Ltd (China), and adult (8-12 weeks old) mice of either sex were used. Mice were maintained with a 12-h light/dark photoperiod (light on at 8 AM) and were provided food and water ad libitum.

### Tissue Sample Preparation for Immunohistochemistry.

Mice were anesthetized with an overdose of pentobarbital and perfused intracardially with PBS followed by 4% paraformaldehyde (PFA; 4% (w/v) in PBS). Brains were dissected out and postfixed in 4% PFA for 4 h at RT. Samples were cryoprotected in 30% sucrose until they sank. Coronal sections (35 μm) were prepared on a Cryostat microtome (Leica CM1950).

Human skin samples were obtained surgically, from 28-year-old female arm skin with psoriasis and 23-year-old male health arm skin. Samples were embedded in O.C.T compound, frozen at −80° C., and sectioned into 30-μm-thick specimens. Skin sections were fixed in 4% PFA for 15 min and washed three times with PBS.

Immunostaining of misHCR/misHCRⁿ.

Detailed information about antibodies and the Nb-HCR initiators (or Nb-SS-HCR initiators) being used were listed in Table 3. Samples were permeabilized with 0.3% Triton X-100 in PBS (PBST), and blocked with blocking buffer (2% BSA, 5 mM EDTA, 0.3% Triton X-100 in PBS) for 1 h at RT. All prepared Ab-HCR initiators (or Ab-SS-HCR initiators) by MaMBA strategy were pooled into incubation buffer (1% BSA, 0.1% Triton X-100, 5 mM EDTA, 0.5 mg/mL sheared Salmon sperm DNA, 1% dextran sulfate, 150 mM NaCl, 0.05% NaN₃) supplemented with excess Nb-NGL-His, according to the nanobody type used above. Sections were then incubated with the Ab-HCR initiators (or Ab-SS-HCR initiators) pool at 4° C. for 12-16 h, and then washed three times with washing buffer (2% BSA, 0.1% Triton X-100 in PBS) for 10 min. Samples were washed twice with PBS for 5 min, and then postfixed using 5 mM BS (PEG)₅ (A35396, Thermo Fisher Scientific) in PBS for 1 h at RT followed by quenching in Tris-buffered saline (TBS) for 10 min. For control experiments, sections were incubated with antibodies diluted in blocking buffer at 4° C. for 12-16 h and then washed three times in PBST. 1), samples were incubated with Nb-fluorophore or Nb-HCR initiator in incubation buffer for 2 h at RT, and then washed three times with PBST. 2), samples were incubated with a mixture containing equal amounts of Cy3-conjugated anti-rabbit IgG antibody (111-165-008, Jackson ImmunoResearch) and Nb-HCR initiator (B4 I1) in incubation buffer for 2 h at RT, and then washed three times with PBST.

Before performing next round of misHCR, tissue sections with the anchored Ab-SS-HCR initiators were incubated with 50 mM TCEP in PBS (to remove HCR initiators via reductive cleavage) for 15 min at RT followed by three washes with PBST.

Samples were blocked in blocking buffer for 1 h at RT, and a new round of staining was performed as above.

Samples of cultured cells were fixed in 4% PFA for 10 min and washed three times with PBST. After blocked with blocking buffer for 30 min at RT, cells were stained by antibody against α-tubulin (1:4,000 diluted; T5168, Sigma-Aldrich) following Alexa Fluor 647-conjugated secondary antibodies (1:1,000 diluted; 715-605-151, Jackson ImmunoResearch), or by equal amounts of the Ab-HCR initiator (B4 I1) against α-tubulin following HCR using Alexa Fluor 647-conjugated amplifiers.

### HCR Amplification.

The basic HCR amplification process was common to all the experiments. Samples were blocked in amplification buffer (5×SSC buffer, 0.1% (v/v) Tween-20, 10% dextran sulfate) at RT for 1 h. Each pair of fluorophore-conjugated HCR amplifiers were snap-cooled separately in 5×SSC buffer by being heated at 95° C. for 90 s using a PCR machine, immediately cooled on ice for 5 min, and then kept in dark at RT over 30 min. Amplifiers were added to amplification buffer to a final concentration of 12.5 nM for each amplifier. Samples were incubated with HCR amplifiers overnight at RT, and free amplifiers were then removed by three washes with 5×SSCT (5×SSC buffer, 0.1% Tween-20) before signal detection.

To perform multi-round imaging, hybridized amplifiers were first removed with 50% formamide in 0.1×PBS at 37° C. for 5 min twice. Samples were then washed three times with PBST and blocked again in amplification buffer for 1 h at RT followed by the basic HCR amplification process.

Hybridized amplifiers should be removed before the HCR initiators removal for a new round of staining.

### Fluorescence Imaging.

Samples were mounted on microscope slide in 50% glycerol containing 1 μg/mL DAPI (D9542, Sigma-Aldrich) for image acquisition. Confocal microscopy was mainly performed on a Zeiss LSM880 using a 20×/NA-0.8 objective, or on a Leica SP8 using 20×/NA-0.75 or 10×/NA-0.40 objective. Alexa Fluor (AF) 488 was visualized with a 488-nm laser; Cy3, AF546 and AF594 was visualized with a 552-nm laser; Cy5 and AF647 was visualized with a 638-nm laser. The images were 1024×1024 pixels or 512×512 pixels and acquired with frame averaging of two. Images were processed using Zeiss ZEN, Leica LAS X and FIJI, and colored for display using FIJI and Photoshop. For multi-round images, a basic alignment and registration was done by descriptor-based registration plugin based on DAPI channel of each image using FIJI.

For 6-color multiplexing of human skin samples, the entire experiment was performed using one stain round and 3 image rounds (round 1, PDGFRα, KRT14 and DCT; round 2, CD31; round 3, αSMA and CD45). For 9-color multiplexing of mouse brain slices, 3 stain rounds with one image round of each were performed (1^{st} misHCR round, MAP2, DDC and nNOS; 2^{nd} misHCR round, NeuN, Iba1 and GFAP; 3^{rd} misHCR round, Th, NF-H and Tph2). For 12-color multiplexing of mouse brain slices, two rounds of misHCR were performed with 4 image rounds (round 1, nNOS and GFAP; round 2, NeuN, Th and NF-H; round 3, Tph2 and Orexin A; round 4, GABA) and 3 image rounds (round 1, Tmem119 and DDC; round 2, 5-HT; round 3, Iba1) respectively.

### Cell Culture and Transfection.

HEK293T cells (ATCC CRL-3216) and HeLa cells (ATCC CCL-2) were incubated according to standard procedures in Dulbecco's modified Eagle medium (DMEM, Gibco) supplemented with 10% (v/v) fetal bovine serum (FBS, Gibco) at 37° C. with 5% CO₂. Cells were seeded in a 24-well plate and grown to 70-90% viable. For immunostaining, cells were cultured on cover slips pretreated with poly-L-lysine solution (P8920, Sigma-Aldrich). Cell medium was replaced with fresh complete medium 2 h before transfection. For each well, 0.5 μg plasmid and 0.5 μL DNA transfection reagent (Neofect) were mixed in 50 μL serum-free medium and incubated for 15-20 min at RT. The mixture was added to the cell culture medium, and cells were incubated for 18-24 h before assaying for transgene expression. For cells transfected with TRE-EGFP-PGK-rtTA, Tet system approved FBS (Biological Industries) was used instead.

### Drug Treatment.

Drug used in this study were anhydrotetracycline hydrochloride (ATc; 541642, J&K), 3-Isobutyl-1-methylxanthine (IBMX; I5879, Sigma-Aldrich) and Forskolin (FSK; F3917, Sigma-Aldrich).

Cells transfected with TRE-EGFP-PGK-rtTA were induced with increasing dose of ATc as described. Cells transfected with 3×CRE-mCherry-hPEST were induced with 0.3 mM IBMX and increasing dose of FSK as described. Cells co-transfected with TRE-EGFP-PGK-rtTA and 3×CRE-mCherry-hPEST were treated with 0.3 mM IBMX, increasing dose of ATc and decreasing dose of FSK as described. After incubation for 6-7 h at 37° C. with 5% CO₂, cells were lysed in cell extraction buffer supplemented with 1 mM PMSF and protease inhibitor cocktail following manufacturer's instructions, quantified using BCA protein assay (23225, Thermo Fisher Scientific) and stored in aliquots at −80° C. until use.

### Serum Sample Collection.

Human serum samples were collected from volunteers of laboratory workers and individuals from routine physical examination for health screening in hospitals, approved by the human research ethics committee of Chinese Institute for Brain Research, Beijing. Serum samples for anti-SARS COV-2 Spike RBD IgG detection were collected in October 2021, while samples for HBV antigens detection were collected in June 2021.

### BLISA Assay on Microplate Surface.

Detailed information about antibodies and the Nb-SS-DNA barcodes being used were listed in Table 2. HRP-conjugated secondary antibodies against rabbit IgG (1:30,000 diluted; 31460, Thermo Fisher Scientific) and mouse IgG (1:30,000 diluted; 31430, Thermo Fisher Scientific) were used.

For samples containing purified GFP and mCherry, 50 μL purified proteins diluted in coating buffer (50 mM carbonate buffer, pH 9.6) containing 1 μg/mL wildtype HEK293t cell lysate were added on a polystyrene microplate (449824, Thermo Fisher Scientific) and incubated for 2 h at 37° C. Wells were aspirated and washed three times with plate wash buffer (5 mM EDTA, 0.05% Tween-20 in PBS). Then, samples were blocked for 1 h at RT in 200 μL blocking buffer (0.05% Tween-20 in PFBB) and washed three times with plate wash buffer. All prepared Ab-SS-DNA barcodes by MaMBA strategy were pooled into incubation solution (blocking buffer supplemented with 5 mM EDTA, 0.4 mg/mL sheared Salmon sperm DNA, 2 μM TP1107-NGL-His 6 and 2 μM TP897-NGL-His₆). Samples were then incubated with the Ab-SS-DNA barcodes pool for 1 h at 37° C. and washed five times with plate wash buffer and three times with PBS containing 5 mM EDTA. DNA barcodes were retrieved with 50 μL elution buffer (20 mM TCEP in PBS) per well and incubated for 0.5 h at RT. The DNA retrieved solutions were measured immediately or stored at −20° C. until analysis. Volume of 1 μL was used as template for qPCR or sequencing library preparation. For ELISA control experiments, samples were incubated with antibody (diluted in PFBB) for 1 h at 37° C. and washed three times with plate wash buffer. Samples were then incubated with HRP-conjugated secondary antibody diluted in PFBB for 1 h at 37° C. and washed five times with plate wash buffer. The HRP-driven colorimetric readout was done by adding 50 μL TMB solution and incubating for 10 min at RT. Then 50 μL 1.8 N H₂SO₄ was added to stop the reaction, and samples were immediately measured absorbance at 450 nm with results subtracted by the value of blank well.

For samples of endogenously expressed GFP and mCherry, 1 μg/mL cell lysate diluted in coating buffer were added into the microplate following the BLISA protocol described above.

For anti-SARS-CoV-2 Spike RBD IgG detection, sandwich immunoassay was adopted by coating SARS-CoV-2 Spike RBD-His recombinant proteins (40592-V08B, Sino Biological) (1 μg/mL diluted in PBS) on the microplate at RT overnight and washing for three times with plate wash buffer by microplate washer (Wellwash™ Versa Microplate Washer, Thermo Fisher Scientific). After incubation in blocking buffer for 1 h at RT following three times of washing, 1 μL serum and 49 μL PBS were added to the coated microplate and incubated for 1 h at 37° C. After five times of washing, captured anti-SARS-CoV-2 Spike RBD IgG was detected by Ab-SS-DNA barcode (antibody against human IgG) diluted in incubation buffer following washing and DNA retrieval as described above.

For HBsAg and HBeAg detection, 40 μL serum, 10 μL PBS and 50 μL Ab-SS-DNA barcodes pool (detection antibodies against HBsAg and HBeAg) were added to the microplate coated with the capture antibodies, following washing and DNA retrieval.

TABLE 3
Antibodies and Nb-DNA oligos (or Nb-SS-DNA oligos).

| | | | | Dilution/Working | Nb-DNA oligos (or |
|---|---|---|---|---|---|
| Epitope | Host | Vendor | Cat. No. | concentration | Nb-SS-DNA oligos) |
| PDGFRα | Rabbit | Abcam | ab203491 | 1:500 | TP897-B1 I1 |
| KRT14 | Rabbit | Ting Chen's Lab | | 1:1000 | TP897-B3 I1 |
| DCT | Rabbit | Ting Chen's Lab | | 1:500 | TP897-B4 I1 |
| CD31 | Mouse | Abcam | ab9498 | 1:500 | TP1107-B9 I1 |
| | (IgG1) | | | | |
| αSMA | Rabbit | Abcam | ab124964 | 1:500 | TP897-B10 I1 |
| CD45 | Mouse | BD Biosciences | 555481 | 1:300 | TP1107-B13 I1 |
| | (IgG1) | | | | |
| MAP2 | Rabbit | Thermo Fisher | PA5-85755 | 1:500 | TP897-B1 I1 |
| | | Scientific | | | |
| NeuN | Rabbit | Abcam | ab177487 | 1:500 | TP897-B1 I1, |
| | | | | | TP897-SS-B15 I1 |
| TPH2 | Rabbit | Abcam | ab184505 | 1:200 | TP897-B5 I1, |
| | | | | | TP897-SS-B5 I1 |
| TH | Rabbit | Merck Millipore | AB152 | 1:500 | TP897-B1 I1, |
| | | | | | TP897-SS-B14 I1 |
| nNOS | Rabbit | Sigma-Aldrich | N7280 | 1:500 | TP897-B5 I1, |
| | | | | | TP897-SS-B10 I1 |
| GFAP | Rabbit | Abcam | ab7260 | 1:750 | TP897-B5 I1, |
| | | | | | TP897-SS-B13 I1 |
| Iba1 | Rabbit | FUJIFILM | 019-19741 | 1:500 | TP897-B4 I1, |
| | | | | | TP897-SS-B13 I1 |
| DDC | Rabbit | Abcam | ab3905 | 1:50 | TP897-B4 I1, |
| | | | | | TP897-SS-B14 I1 |
| NF-H | Rabbit | Thermo Fisher | 711025 | 1:100 | TP897-B4 I1, |
| | | Scientific | | | TP897-SS-B17 I1 |
| TMEM119 | Rabbit | Abcam | ab209064 | 1:500 | TP897-B1 I1, |
| | | | | | TP897-SS-B15 I1 |
| GABA | Rabbit | Sigma-Aldrich | A2025 | 1:750 | TP897-B1 I1, |
| | | | | | TP897-SS-B2 I1 |
| Orexin A | Rabbit | Phoenix | H-003-30 | 1:500 | TP897-B1 I1, |
| | | Pharmaceuticals | | | TP897-SS-B9 I1 |
| 5-HT | Rabbit | Immunostar | 20080 | 1:500 | TP897-B1 I1, |
| | | | | | TP897-SS-B4 I1 |
| α-tubulin | Mouse | Sigma-Aldrich | T5168 | 1:40000 | TP1107-SS-qbc. 1, |
| | (IgG1) | | | | TP1107-SS-sbc. 1 |
| GFP | Rabbit | Thermo Fisher | A-11122 | 1:1000 | TP897-SS-qbc. 2, |
| | | Scientific | | | TP897-SS -sbc. 2 |
| mCherry | Rabbit | Thermo Fisher | PA5-34974 | 1:2000 for | TP897-SS-qbc. 3, |
| | | Scientific | | purified | TP897-SS-sbc. 3 |
| | | | | mCherry, | |
| | | | | 1:10000 for | |
| | | | | endogenous | |
| | | | | mCherry | |
| Human IgG | Rabbit | Abcam | ab181236 | 0.33 nM | TP897-SS-qbc. 2, |
| | | | | | TP897-SS-sbc. 4 |
| HBsAg (capture) | Goat | Beijing Wantai | YTX2101 | 5 μg/mL | |
| | | Biological | | | |
| HBsAg (detection) | Mouse | Beijing Wantai | HBs-2C1 | 0.33 nM | TP1107-SS-qbc. 3, |
| | (IgG1) | Biological | | | TP1107-SS-sbc. 3 |
| HBeAg (capture) | Mouse | Beijing Wantai | 13B12-1 | 5 μg/mL | |
| | (IgG2a) | Biological | | | |
| HBeAg (detection) | Mouse | Beijing Wantai | 9A4-1 | 0.033 nM | TP1107-SS-qbc. 2, |
| | (IgG1) | Biological | | | TP1107-SS-sbc. 2 |
| phospho-p38α (T180/Y182) | Mouse | R&D Systems | DYC869B | | |
| (capture) | | | | | |
| phospho-p38aα (T180/Y182) | Rabbit | R&D Systems | DYC869B | | TP897-SS-qbc. 2, |
| (detection) | | | | | TP897-SS-sbc. 2 |
| phospho-ERK1 | Mouse | R&D Systems | DYC1018B | | |
| (T202/Y204)/ERK2 (T185/Y187) | | | | | |
| (capture) | | | | | |
| phospho-ERK1 | Rabbit | R&D Systems | DYC1018B | | TP897-SS-qbc. 3, |
| (T202/Y204)/ERK2(T185/Y187) | | | | | TP897-SS-sbc. 3 |
| (detection) | | | | | |
| phospho-JNK Pan Specific | Mouse | R&D Systems | DYC1387B | | |
| (capture) | | | | | |
| phospho-JNK Pan Specific | Rabbit | R&D Systems | DYC1387B | | TP897-SS-qbc. 4, |
| (detection) | | | | | TP897-SS-sbc. 4 |
| phospho-AMPKα1 (T183) | Goat | R&D Systems | DYC3528 | | |
| (capture) | | | | | |
| phospho-AMPKα1 (T183) | Rabbit | R&D Systems | DYC3528 | | TP897-SS-qbc. 5, |
| (detection) | | | | | TP897-SS-sbc. 5 |
| phospho-CREB (S133) (capture) | Goat | R&D Systems | DYC2510 | | |
| phospho-CREB (S133) (detection) | Rabbit | R&D Systems | DYC2510 | | TP897-SS-qbc. 6, |
| | | | | | TP897-SS-sbc. 6 |
| phospho-Src (Y419) (capture) | Goat | R&D Systems | DYC2685 | | |
| phospho-Src (Y419) (detection) | Rabbit | R&D Systems | DYC2685 | | TP897-SS-qbc. 7, |
| | | | | | TP897-SS-sbc. 7 |
| phospho-Akt (S473) (capture) | Mouse | R&D Systems | DYC887B | | |
| phospho-Akt (S473) (detection) | Mouse | R&D Systems | DYC887B | | TP1107-SS-qbc. 1, |
| | (IgG1) | | | | TP1107-SS-sbc. 1 |
| GAPDH (capture) | Rabbit | Abcam | ab252636 | | |
| GAPDH (detection) | Rabbit | Abcam | ab276013 | | TP897-SS-qbc. 8, |
| | | | | | TP897-SS-sbc. 8 |

### BLISA Assay on Magnetic Beads.

Detailed information about the antibody pairs and DNA barcodes were listed in Table 3. Briefly, each vial of capture antibody was coupled to 18 mg Epoxy magnetic beads (Beijing Yunci Technology). Beads were mixed with 0.1 M sodium phosphate (pH 7.4), capture antibody and 1 M ammonium sulfate. The mixtures were incubated for 16-24 h at 37° C. with rotation (5 rpm). Then beads were washed three times with PBS containing 1% Triton X-100, followed by blocking in PBS with 1% BSA and 0.05% Tween-20 for 2 h at RT. After magnetic isolation, beads were maintained in PBS with 0.1% BSA, 0.05% Tween-20 and 0.03% NaN₃ at a concentration of 10 mg/mL, and stored at 4° C. For multiplex captures, the antibody-coupled beads for all targets were mixed before use.

After rinsed twice with beads wash buffer (0.1% Triton X-100 in PBS), beads were resuspended in wash buffer and distributed into each well of a 96-well deep well plate (501102, NEST). Then beads were mixed with 100 μL cell lysates samples or standard proteins for 30 min at RT in a plate mixer (800 rpm). The beads were isolated using a 96-well magnetic stand following 3× wash steps with beads wash buffer. The antigen-captured beads were detected by Ab-SS-DNA barcodes pooled in beads incubation buffer (0.5% BSA, 0.05% Triton X-100, 0.1% dextran sulfate, 0.4 mg/mL sheared Salmon sperm DNA, 5 μM TP1107-NGL-His₆, 2 μM TP897-NGL-His, in PBS) for 30 min at RT in a plate mixer (800 rpm), followed by 5× wash steps with beads wash buffer. Beads were rinsed twice with PBS, and DNA barcodes were retrieved with 50 μL elution buffer (20 mM TCEP in PBS) for 5-10 min at RT in a plate mixer (1000 rpm). After magnetic isolation, the DNA retrieved solutions were transferred to new tubes, and measured immediately or stored at −20° C. until analysis.

### Design of DNA Barcode Sequences.

The DNA barcode sequences (62 bp) were designed to include a 6-bp antibody-dedicated barcode (Ab bc) and a 15-bp unique molecular identifier (UMI). For qPCR, DNA barcodes are designed with different both-end sequences for specific amplification using different pairs of primers. The sequences of all DNA barcodes were listed in Table 1.

Quantitative PCR (qPCR) Measurement.

After thorough vortexing, 1 μL of DNA retrieved solution was used for qPCR (20 μL/reaction, triplicates for each sample well) with SYBR qPCR master mix (Q712, Vazyme) in CFX 96 machine (Bio-rad).

### Sequencing Library Preparation.

For the two-step PCR protocol to further barcode the released DNA barcodes of each well, a 25 μL PCR was performed per sample containing 1 μL released DNA barcodes, 0.1 mM dNTPs, 0.25 μL Q5 high-fidelity DNA polymerase (NEB), 1×Q5 reaction buffer, 0.4 μM well-specific forward primer (6-bp well barcode) and 0.4 μM reverse primer (Tables 4-6). PCR thermocycling conditions were performed as followed (PCR condition 1): (1) 30 s at 98° C.; (2) 10 s at 98° C.; (3) 30 s at 50° C.; (4) 10 s at 72° C.; (5) repeat step 2-4 nine times; (6) 2 min at 72° C.; and (7)∞12° C. 1 μL of each well-barcoded DNA from one plate were pooled and purified using 1.8×AMPure XP beads (Beckman Coulter) following the manufacturer's protocol. The well-barcoded DNA pool was eluted with 30 μL nuclease-free water. Another 50 μL PCR was prepared with 29 μL eluted DNA, 0.2 mM dNTPs, 0.5 μL Q5 polymerase, 1×Q5 reaction buffer, 0.5 μM adaptor forward primer (5′-AATGATACGGCGACCACCGAGATCTACACTCTTTCCCTACACGACG-3′, SEQ ID NO: 63) and 0.5 μM adaptor reverse primer containing i7 index (5′-CAAGCAGAAGACGGCATACGAGATGCCTAAGTGACTGGAGTTCAGACG-3′, SEQ ID NO: 64). PCR condition 1 was performed and the products were purified by 1.8×AMPure XP beads. The final sequencing library was eluted in 30 μL nuclease-free water.

For the three-step PCR protocol with phasing spacers addition, a 25 μL PCR was performed per well containing 1 μL DNA barcodes (released DNA and norm. bc.), 0.1 mM dNTPs, 0.25 μL Q5 high-fidelity DNA polymerase, 1×Q5 reaction buffer, 0.5 μM well-specific forward primer, and 0.5 μM reverse primer (Table 4). PCR condition 1 was performed. 0.73 μL of each well-barcoded DNA from the same plate were pooled and purified by 1.8×AMPure XP beads with elution in 85 μL nuclease-free water. Then the eluted well-barcoded DNA pool was separated (with 10 μL of each) to 8 PCR reactions (25 μL) for phasing spacers addition containing 0.1 mM dNTPs, 0.25 μL Q5 high-fidelity DNA polymerase, 1×Q5 reaction buffer, 0.5 μM PS forward primer, and 0.5 μM PS reverse primer (Table 4). PCR thermocycling conditions were performed as followed: (1) 30 s at 98° C.; (2) 10 s at 98° C.; (3) 30 s at 55° C.; (4) 10 s at 72° C.; (5) repeat step 2-4 nine times; (6) 2 min at 72° C.; and (7) ∞12° C. 8.75 μL of each phasing spacer-tagged DNA product were pooled (70 μL) and purified by 1.8×AMPure XP beads with elution in 30 μL nuclease-free water. Then 50 μL PCR was prepared with 5 μL eluted DNA, 0.2 mM dNTPs, 0.5 μL Q5 polymerase, 1×Q5 reaction buffer, 0.5 μM forward primer 151AR containing i5 index and 0.5 μM reverse primer 117JvA containing i7 index (plate index). PCR thermocycling conditions were performed as followed: (1) 30 s at 98° C.; (2) 10 s at 98° C.; (3) 30 s at 60° C.; (4) 10 s at 72° C.; (5) repeat step 2-4 nine times; (6) 2 min at 72° C.; and (7) ∞12° C. The adaptor-tailed DNA libraries were purified by 1.8×AMPure XP beads and eluted with 20 μL nuclease-free water. Each plate library was analyzed and quantified to pool together for sequencing.

Sequencing was performed on a HiSeq2500 System with manufacturer's instructions (Illumina).

TABLE 4
Two-step PCR (step 1 primers)

| | | | Length |
|---|---|---|---|
| | | well bc.-F whole sequence | (bp) |
| Forward | well | CTCTTTCCCTACACGACGCTCTTCCGATCTATCACGCTTGTGGAAAGGAC | 56 |
| Primer | bc.-F1 | GAAACA (SEQ ID NO: 65) | |
| | well | CTCTTTCCCTACACGACGCTCTTCCGATCTCGATGTCTTGTGGAAAGGAC | 56 |
| | bc.-F2 | GAAACA (SEQ ID NO: 66) | |
| | well | CTCTTTCCCTACACGACGCTCTTCCGATCTTTAGGCCTTGTGGAAAGGAC | 56 |
| | bc.-F3 | GAAACA (SEQ ID NO: 67) | |
| | well | CTCTTTCCCTACACGACGCTCTTCCGATCTTGACCACTTGTGGAAAGGAC | 56 |
| | bc.-F4 | GAAACA (SEQ ID NO: 68) | |
| | well | CTCTTTCCCTACACGACGCTCTTCCGATCTACAGTGCTTGTGGAAAGGAC | 56 |
| | bc.-F5 | GAAACA (SEQ ID NO: 69) | |
| | well | CTCTTTCCCTACACGACGCTCTTCCGATCTGCCAATCTTGTGGAAAGGAC | 56 |
| | bc.-F6 | GAAACA (SEQ ID NO: 70) | |
| | well | CTCTTTCCCTACACGACGCTCTTCCGATCTCAGATCCTTGTGGAAAGGAC | 56 |
| | bc.-F7 | GAAACA (SEQ ID NO: 71) | |
| | well | CTCTTTCCCTACACGACGCTCTTCCGATCTACTTGACTTGTGGAAAGGAC | 56 |
| | bc.-F8 | GAAACA (SEQ ID NO: 72) | |
| | well | CTCTTTCCCTACACGACGCTCTTCCGATCTGATCAGCTTGTGGAAAGGAC | 56 |
| | bc.-F9 | GAAACA (SEQ ID NO: 73) | |
| | well | CTCTTTCCCTACACGACGCTCTTCCGATCTTAGCTTCTTGTGGAAAGGAC | 56 |
| | bc.-F10 | GAAACA (SEQ ID NO: 74) | |
| | well | CTCTTTCCCTACACGACGCTCTTCCGATCTGGCTACCTTGTGGAAAGGAC | 56 |
| | bc.-F11 | GAAACA (SEQ ID NO: 75) | |
| | well | CTCTTTCCCTACACGACGCTCTTCCGATCTCTTGTACTTGTGGAAAGGAC | 56 |
| | bc.-F12 | GAAACA (SEQ ID NO: 76) | |
| | well | CTCTTTCCCTACACGACGCTCTTCCGATCTAGTCAACTTGTGGAAAGGAC | 56 |
| | bc.-F13 | GAAACA (SEQ ID NO: 77) | |
| | well | CTCTTTCCCTACACGACGCTCTTCCGATCTAGTTCCCTTGTGGAAAGGAC | 56 |
| | bc.-F14 | GAAACA (SEQ ID NO: 78) | |
| | well | CTCTTTCCCTACACGACGCTCTTCCGATCTATGTCACTTGTGGAAAGGAC | 56 |
| | bc.-F15 | GAAACA (SEQ ID NO: 79) | |
| | well | CTCTTTCCCTACACGACGCTCTTCCGATCTCCGTCCCTTGTGGAAAGGAC | 56 |
| | bc.-F16 | GAAACA (SEQ ID NO: 80) | |
| | well | CTCTTTCCCTACACGACGCTCTTCCGATCTGTAGAGCTTGTGGAAAGGAC | 56 |
| | bc.-F17 | GAAACA (SEQ ID NO: 81) | |
| | well | CTCTTTCCCTACACGACGCTCTTCCGATCTGTCCGCCTTGTGGAAAGGAC | 56 |
| | bc.-F18 | GAAACA (SEQ ID NO: 82) | |
| | well | CTCTTTCCCTACACGACGCTCTTCCGATCTGTGAAACTTGTGGAAAGGAC | 56 |
| | bc.-F19 | GAAACA (SEQ ID NO: 83) | |
| | well | CTCTTTCCCTACACGACGCTCTTCCGATCTGTGGCCCTTGTGGAAAGGAC | 56 |
| | bc.-F20 | GAAACA (SEQ ID NO: 84) | |
| | well | CTCTTTCCCTACACGACGCTCTTCCGATCTGTTTCGCTTGTGGAAAGGAC | 56 |
| | bc.-F21 | GAAACA (SEQ ID NO: 85) | |
| | well | CTCTTTCCCTACACGACGCTCTTCCGATCTCGTACGCTTGTGGAAAGGAC | 56 |
| | bc.-F22 | GAAACA (SEQ ID NO: 86) | |
| | well | CTCTTTCCCTACACGACGCTCTTCCGATCTGAGTGGCTTGTGGAAAGGAC | 56 |
| | bc.-F23 | GAAACA (SEQ ID NO: 87) | |
| | well | CTCTTTCCCTACACGACGCTCTTCCGATCTGGTAGCCTTGTGGAAAGGAC | 56 |
| | bc.-F24 | GAAACA (SEQ ID NO: 88) | |
| | well | CTCTTTCCCTACACGACGCTCTTCCGATCTACTGATCTTGTGGAAAGGAC | 56 |
| | bc.-F25 | GAAACA (SEQ ID NO: 89) | |
| | well | CTCTTTCCCTACACGACGCTCTTCCGATCTATGAGCCTTGTGGAAAGGAC | 56 |
| | bc.-F26 | GAAACA (SEQ ID NO: 90) | |
| | well | CTCTTTCCCTACACGACGCTCTTCCGATCTATTCCTCTTGTGGAAAGGAC | 56 |
| | bc.-F27 | GAAACA (SEQ ID NO: 91) | |
| | well | CTCTTTCCCTACACGACGCTCTTCCGATCTCAAAAGCTTGTGGAAAGGAC | 56 |
| | bc.-F28 | GAAACA (SEQ ID NO: 92) | |
| | well | CTCTTTCCCTACACGACGCTCTTCCGATCTCAACTACTTGTGGAAAGGAC | 56 |
| | bc.-F29 | GAAACA (SEQ ID NO: 93) | |
| | well | CTCTTTCCCTACACGACGCTCTTCCGATCTCACCGGCTTGTGGAAAGGAC | 56 |
| | bc.-F30 | GAAACA (SEQ ID NO: 94) | |
| | well | CTCTTTCCCTACACGACGCTCTTCCGATCTCACGATCTTGTGGAAAGGAC | 56 |
| | bc.-F31 | GAAACA (SEQ ID NO: 95) | |
| | well | CTCTTTCCCTACACGACGCTCTTCCGATCTCACTCACTTGTGGAAAGGAC | 56 |
| | bc.-F32 | GAAACA (SEQ ID NO: 96) | |
| | well | CTCTTTCCCTACACGACGCTCTTCCGATCTCAGGCGCTTGTGGAAAGGAC | 56 |
| | bc.-F33 | GAAACA (SEQ ID NO: 97) | |
| | well | CTCTTTCCCTACACGACGCTCTTCCGATCTCATGGCCTTGTGGAAAGGAC | 56 |
| | bc.-F34 | GAAACA (SEQ ID NO: 98) | |
| | well | CTCTTTCCCTACACGACGCTCTTCCGATCTCATTTTCTTGTGGAAAGGAC | 56 |
| | bc.-F35 | GAAACA (SEQ ID NO: 99) | |
| | well | CTCTTTCCCTACACGACGCTCTTCCGATCTCCAACACTTGTGGAAAGGAC | 56 |
| | bc.-F36 | GAAACA (SEQ ID NO: 100) | |
| | well | CTCTTTCCCTACACGACGCTCTTCCGATCTCGGAATCTTGTGGAAAGGAC | 56 |
| | bc.-F37 | GAAACA (SEQ ID NO: 101) | |
| | well | CTCTTTCCCTACACGACGCTCTTCCGATCTCTAGCTCTTGTGGAAAGGAC | 56 |
| | bc.-F38 | GAAACA (SEQ ID NO: 102) | |
| | well | CTCTTTCCCTACACGACGCTCTTCCGATCTCTATACCTTGTGGAAAGGAC | 56 |
| | bc.-F39 | GAAACA (SEQ ID NO: 103) | |
| | well | CTCTTTCCCTACACGACGCTCTTCCGATCTCTCAGACTTGTGGAAAGGAC | 56 |
| | bc.-F40 | GAAACA (SEQ ID NO: 104) | |
| | well | CTCTTTCCCTACACGACGCTCTTCCGATCTGACGACCTTGTGGAAAGGAC | 56 |
| | bc.-F41 | GAAACA (SEQ ID NO: 105) | |
| | well | CTCTTTCCCTACACGACGCTCTTCCGATCTTAATCGCTTGTGGAAAGGAC | 56 |
| | bc.-F42 | GAAACA (SEQ ID NO: 106) | |
| | well | CTCTTTCCCTACACGACGCTCTTCCGATCTTACAGCCTTGTGGAAAGGAC | 56 |
| | bc.-F43 | GAAACA (SEQ ID NO: 107) | |
| | well | CTCTTTCCCTACACGACGCTCTTCCGATCTTATAATCTTGTGGAAAGGAC | 56 |
| | bc.-F44 | GAAACA (SEQ ID NO: 108) | |
| | well | CTCTTTCCCTACACGACGCTCTTCCGATCTTCATTCCTTGTGGAAAGGAC | 56 |
| | bc.-F45 | GAAACA (SEQ ID NO: 109) | |
| | well | CTCTTTCCCTACACGACGCTCTTCCGATCTTCCCGACTTGTGGAAAGGAC | 56 |
| | bc.-F46 | GAAACA (SEQ ID NO: 110) | |
| | well | CTCTTTCCCTACACGACGCTCTTCCGATCTTCGAAGCTTGTGGAAAGGAC | 56 |
| | bc.-F47 | GAAACA (SEQ ID NO: 111) | |
| | well | CTCTTTCCCTACACGACGCTCTTCCGATCTTCGGCACTTGTGGAAAGGAC | 56 |
| | bc.-F48 | GAAACA (SEQ ID NO: 112) | |
| | well | CTCTTTCCCTACACGACGCTCTTCCGATCTGTACTTCTTGTGGAAAGGAC | 56 |
| | bc.-F49 | GAAACA (SEQ ID NO: 113) | |
| | well | CTCTTTCCCTACACGACGCTCTTCCGATCTATGGCACTTGTGGAAAGGAC | 56 |
| | bc.-F50 | GAAACA (SEQ ID NO: 114) | |
| | well | CTCTTTCCCTACACGACGCTCTTCCGATCTAAGGAGCTTGTGGAAAGGAC | 56 |
| | bc.-F51 | GAAACA (SEQ ID NO: 115) | |
| | well | CTCTTTCCCTACACGACGCTCTTCCGATCTCTATCTCTTGTGGAAAGGAC | 56 |
| | bc.-F52 | GAAACA (SEQ ID NO: 116) | |
| | well | CTCTTTCCCTACACGACGCTCTTCCGATCTTATATCCTTGTGGAAAGGAC | 56 |
| | bc.-F53 | GAAACA (SEQ ID NO: 117) | |
| | well | CTCTTTCCCTACACGACGCTCTTCCGATCTTATTATCTTGTGGAAAGGAC | 56 |
| | bc.-F54 | GAAACA (SEQ ID NO: 118) | |
| | well | CTCTTTCCCTACACGACGCTCTTCCGATCTAGCACCCTTGTGGAAAGGAC | 56 |
| | bc.-F55 | GAAACA (SEQ ID NO: 119) | |
| | well | CTCTTTCCCTACACGACGCTCTTCCGATCTTCCACACTTGTGGAAAGGAC | 56 |
| | bc.-F56 | GAAACA (SEQ ID NO: 120) | |
| | well | CTCTTTCCCTACACGACGCTCTTCCGATCTTTCTCGCTTGTGGAAAGGAC | 56 |
| | bc.-F57 | GAAACA (SEQ ID NO: 121) | |
| | well | CTCTTTCCCTACACGACGCTCTTCCGATCTTCCTGCCTTGTGGAAAGGAC | 56 |
| | bc.-F58 | GAAACA (SEQ ID NO: 122) | |
| | well | CTCTTTCCCTACACGACGCTCTTCCGATCTCGCGGCCTTGTGGAAAGGAC | 56 |
| | bc.-F59 | GAAACA (SEQ ID NO: 123) | |
| | well | CTCTTTCCCTACACGACGCTCTTCCGATCTTGCGCTCTTGTGGAAAGGAC | 56 |
| | bc.-F60 | GAAACA (SEQ ID NO: 124) | |
| | well | CTCTTTCCCTACACGACGCTCTTCCGATCTAACGGACTTGTGGAAAGGAC | 56 |
| | bc.-F61 | GAAACA (SEQ ID NO: 125) | |
| | well | CTCTTTCCCTACACGACGCTCTTCCGATCTGACGATCTTGTGGAAAGGAC | 56 |
| | bc.-F62 | GAAACA (SEQ ID NO: 126) | |
| | well | CTCTTTCCCTACACGACGCTCTTCCGATCTTTGCGGCTTGTGGAAAGGAC | 56 |
| | bc.-F63 | GAAACA (SEQ ID NO: 127) | |
| | well | CTCTTTCCCTACACGACGCTCTTCCGATCTCAAGTTCTTGTGGAAAGGAC | 56 |
| | bc.-F64 | GAAACA (SEQ ID NO: 128) | |
| | well | CTCTTTCCCTACACGACGCTCTTCCGATCTTACCTTCTTGTGGAAAGGAC | 56 |
| | bc.-F65 | GAAACA (SEQ ID NO: 129) | |
| | well | CTCTTTCCCTACACGACGCTCTTCCGATCTGAGCGCCTTGTGGAAAGGAC | 56 |
| | bc.-F66 | GAAACA (SEQ ID NO: 130) | |
| | well | CTCTTTCCCTACACGACGCTCTTCCGATCTACGAGGCTTGTGGAAAGGAC | 56 |
| | bc.-F67 | GAAACA (SEQ ID NO: 131) | |
| | well | CTCTTTCCCTACACGACGCTCTTCCGATCTAATCGTCTTGTGGAAAGGAC | 56 |
| | bc.-F68 | GAAACA (SEQ ID NO: 132) | |
| | well | CTCTTTCCCTACACGACGCTCTTCCGATCTGAGTCTCTTGTGGAAAGGAC | 56 |
| | bc.-F69 | GAAACA (SEQ ID NO: 133) | |
| | well | CTCTTTCCCTACACGACGCTCTTCCGATCTAGACCACTTGTGGAAAGGAC | 56 |
| | bc.-F70 | GAAACA (SEQ ID NO: 134) | |
| | well | CTCTTTCCCTACACGACGCTCTTCCGATCTAATTCCCTTGTGGAAAGGAC | 56 |
| | bc.-F71 | GAAACA (SEQ ID NO: 135) | |
| | well | CTCTTTCCCTACACGACGCTCTTCCGATCTAGATGTCTTGTGGAAAGGAC | 56 |
| | bc.-F72 | GAAACA (SEQ ID NO: 136) | |
| | well | CTCTTTCCCTACACGACGCTCTTCCGATCTGGAGAACTTGTGGAAAGGAC | 56 |
| | bc.-F73 | GAAACA (SEQ ID NO: 137) | |
| | well | CTCTTTCCCTACACGACGCTCTTCCGATCTATAGATCTTGTGGAAAGGAC | 56 |
| | bc.-F74 | GAAACA (SEQ ID NO: 138) | |
| | well | CTCTTTCCCTACACGACGCTCTTCCGATCTTATGCACTTGTGGAAAGGAC | 56 |
| | bc.-F75 | GAAACA (SEQ ID NO: 139) | |
| | well | CTCTTTCCCTACACGACGCTCTTCCGATCTCCTATTCTTGTGGAAAGGAC | 56 |
| | bc.-F76 | GAAACA (SEQ ID NO: 140) | |
| | well | CTCTTTCCCTACACGACGCTCTTCCGATCTTAACCGCTTGTGGAAAGGAC | 56 |
| | bc.-F77 | GAAACA (SEQ ID NO: 141) | |
| | well | CTCTTTCCCTACACGACGCTCTTCCGATCTAGTCTCCTTGTGGAAAGGAC | 56 |
| | bc.-F78 | GAAACA (SEQ ID NO: 142) | |
| | well | CTCTTTCCCTACACGACGCTCTTCCGATCTTGGAGCCTTGTGGAAAGGAC | 56 |
| | bc.-F79 | GAAACA (SEQ ID NO: 143) | |
| | well | CTCTTTCCCTACACGACGCTCTTCCGATCTCTGCATCTTGTGGAAAGGAC | 56 |
| | bc.-F80 | GAAACA (SEQ ID NO: 144) | |
| | well | CTCTTTCCCTACACGACGCTCTTCCGATCTCAGGCCCTTGTGGAAAGGAC | 56 |
| | bc.-F81 | GAAACA (SEQ ID NO: 145) | |
| | well | CTCTTTCCCTACACGACGCTCTTCCGATCTCAGTAACTTGTGGAAAGGAC | 56 |
| | bc.-F82 | GAAACA (SEQ ID NO: 146) | |
| | well | CTCTTTCCCTACACGACGCTCTTCCGATCTGTTACCCTTGTGGAAAGGAC | 56 |
| | bc.-F83 | GAAACA (SEQ ID NO: 147) | |
| | well | CTCTTTCCCTACACGACGCTCTTCCGATCTACTGCTCTTGTGGAAAGGAC | 56 |
| | bc.-F84 | GAAACA (SEQ ID NO: 148) | |
| | well | CTCTTTCCCTACACGACGCTCTTCCGATCTCTAAGGCTTGTGGAAAGGAC | 56 |
| | bc.-F85 | GAAACA (SEQ ID NO: 149) | |
| | well | CTCTTTCCCTACACGACGCTCTTCCGATCTATCCAACTTGTGGAAAGGAC | 56 |
| | bc.-F86 | GAAACA (SEQ ID NO: 150) | |
| | well | CTCTTTCCCTACACGACGCTCTTCCGATCTGTGAGACTTGTGGAAAGGAC | 56 |
| | bc.-F87 | GAAACA (SEQ ID NO: 151) | |
| | well | CTCTTTCCCTACACGACGCTCTTCCGATCTTAAGACCTTGTGGAAAGGAC | 56 |
| | bc.-F88 | GAAACA (SEQ ID NO: 152) | |
| | well | CTCTTTCCCTACACGACGCTCTTCCGATCTCGTTAGCTTGTGGAAAGGAC | 56 |
| | bc.-F89 | GAAACA (SEQ ID NO: 153) | |
| | well | CTCTTTCCCTACACGACGCTCTTCCGATCTGTGTATCTTGTGGAAAGGAC | 56 |
| | bc.-F90 | GAAACA (SEQ ID NO: 154) | |
| | well | CTCTTTCCCTACACGACGCTCTTCCGATCTGAATGCCTTGTGGAAAGGAC | 56 |
| | bc.-F91 | GAAACA (SEQ ID NO: 155) | |
| | well | CTCTTTCCCTACACGACGCTCTTCCGATCTAGAGTACTTGTGGAAAGGAC | 56 |
| | bc.-F92 | GAAACA (SEQ ID NO: 156) | |
| | well | CTCTTTCCCTACACGACGCTCTTCCGATCTAGTCCACTTGTGGAAAGGAC | 56 |
| | bc.-F93 | GAAACA (SEQ ID NO: 157) | |
| | well | CTCTTTCCCTACACGACGCTCTTCCGATCTCTATGGCTTGTGGAAAGGAC | 56 |
| | bc.-F94 | GAAACA (SEQ ID NO: 158) | |
| | well | CTCTTTCCCTACACGACGCTCTTCCGATCTACCTTACTTGTGGAAAGGAC | 56 |
| | bc.-F95 | GAAACA (SEQ ID NO: 159) | |
| | well. | CTCTTTCCCTACACGACGCTCTTCCGATCTCGCCAACTTGTGGAAAGGAC | 56 |
| | bc.-F96 | GAAACA (SEQ ID NO: 160) | |
| Reverse | well. | GTGACTGGAGTTCAGACGTGTGCTCTTCCGATCTCTAAAGCGCATGCTC | 54 |
| primer | bc.-R | CAGAC (SEQ ID NO: 161) | |

TABLE 5
Three-step PCR (step 1 primers)

| | Forward | | Length |
|---|---|---|---|
| | Primer | well bc.-F whole sequence | (bp) |
| Forward | well bc.-F1 | GACAAGTGGCCACAAACCACCAGATCACGCTTGTGGAAAGGAC | 49 |
| Primer | | GAAACA (SEQ ID NO: 162) | |
| | well bc.-F2 | GACAAGTGGCCACAAACCACCAGCGATGTCTTGTGGAAAGGAC | 49 |
| | | GAAACA (SEQ ID NO: 163) | |
| | well bc.-F3 | GACAAGTGGCCACAAACCACCAGTTAGGCCTTGTGGAAAGGAC | 49 |
| | | GAAACA (SEQ ID NO: 164) | |
| | well bc.-F4 | GACAAGTGGCCACAAACCACCAGTGACCACTTGTGGAAAGGAC | 49 |
| | | GAAACA (SEQ ID NO: 165) | |
| | well bc.-F5 | GACAAGTGGCCACAAACCACCAGACAGTGCTTGTGGAAAGGAC | 49 |
| | | GAAACA (SEQ ID NO: 166) | |
| | well bc.-F6 | GACAAGTGGCCACAAACCACCAGGCCAATCTTGTGGAAAGGAC | 49 |
| | | GAAACA (SEQ ID NO: 167) | |
| | well bc.-F7 | GACAAGTGGCCACAAACCACCAGCAGATCCTTGTGGAAAGGAC | 49 |
| | | GAAACA (SEQ ID NO: 168) | |
| | well bc.-F8 | GACAAGTGGCCACAAACCACCAGACTTGACTTGTGGAAAGGAC | 49 |
| | | GAAACA (SEQ ID NO: 169) | |
| | well bc.-F9 | GACAAGTGGCCACAAACCACCAGGATCAGCTTGTGGAAAGGAC | 49 |
| | | GAAACA (SEQ ID NO: 170) | |
| | well bc.-F10 | GACAAGTGGCCACAAACCACCAGTAGCTTCTTGTGGAAAGGAC | 49 |
| | | GAAACA (SEQ ID NO: 171) | |
| | well bc.-F11 | GACAAGTGGCCACAAACCACCAGGGCTACCTTGTGGAAAGGAC | 49 |
| | | GAAACA (SEQ ID NO: 172) | |
| | well bc.-F12 | GACAAGTGGCCACAAACCACCAGCTTGTACTTGTGGAAAGGAC | 49 |
| | | GAAACA (SEQ ID NO: 173) | |
| | well bc.-F13 | GACAAGTGGCCACAAACCACCAGAGTCAACTTGTGGAAAGGAC | 49 |
| | | GAAACA (SEQ ID NO: 174) | |
| | well bc.-F14 | GACAAGTGGCCACAAACCACCAGAGTTCCCTTGTGGAAAGGAC | 49 |
| | | GAAACA (SEQ ID NO: 175) | |
| | well bc.-F15 | GACAAGTGGCCACAAACCACCAGATGTCACTTGTGGAAAGGAC | 49 |
| | | GAAACA (SEQ ID NO: 176) | |
| | well bc.-F16 | GACAAGTGGCCACAAACCACCAGCCGTCCCTTGTGGAAAGGAC | 49 |
| | | GAAACA (SEQ ID NO: 177) | |
| | well bc.-F17 | GACAAGTGGCCACAAACCACCAGGTAGAGCTTGTGGAAAGGA | 49 |
| | | CGAAACA (SEQ ID NO: 178) | |
| | well bc.-F18 | GACAAGTGGCCACAAACCACCAGGTCCGCCTTGTGGAAAGGAC | 49 |
| | | GAAACA (SEQ ID NO: 179) | |
| | well bc.-F19 | GACAAGTGGCCACAAACCACCAGGTGAAACTTGTGGAAAGGA | 49 |
| | | CGAAACA (SEQ ID NO: 180) | |
| | well bc.-F20 | GACAAGTGGCCACAAACCACCAGGTGGCCCTTGTGGAAAGGAC | 49 |
| | | GAAACA (SEQ ID NO: 181) | |
| | well bc.-F21 | GACAAGTGGCCACAAACCACCAGGTTTCGCTTGTGGAAAGGAC | 49 |
| | | GAAACA (SEQ ID NO: 182) | |
| | well bc.-F22 | GACAAGTGGCCACAAACCACCAGCGTACGCTTGTGGAAAGGAC | 49 |
| | | GAAACA (SEQ ID NO: 183) | |
| | well bc.-F23 | GACAAGTGGCCACAAACCACCAGGAGTGGCTTGTGGAAAGGA | 49 |
| | | CGAAACA (SEQ ID NO: 184) | |
| | well bc.-F24 | GACAAGTGGCCACAAACCACCAGGGTAGCCTTGTGGAAAGGAC | 49 |
| | | GAAACA (SEQ ID NO: 185) | |
| | well bc.-F25 | GACAAGTGGCCACAAACCACCAGACTGATCTTGTGGAAAGGAC | 49 |
| | | GAAACA (SEQ ID NO: 186) | |
| | well bc.-F26 | GACAAGTGGCCACAAACCACCAGATGAGCCTTGTGGAAAGGAC | 49 |
| | | GAAACA (SEQ ID NO: 187) | |
| | well bc.-F27 | GACAAGTGGCCACAAACCACCAGATTCCTCTTGTGGAAAGGAC | 49 |
| | | GAAACA (SEQ ID NO: 188) | |
| | well bc.-F28 | GACAAGTGGCCACAAACCACCAGCAAAAGCTTGTGGAAAGGA | 49 |
| | | CGAAACA (SEQ ID NO: 189) | |
| | well bc.-F29 | GACAAGTGGCCACAAACCACCAGCAACTACTTGTGGAAAGGAC | 49 |
| | | GAAACA (SEQ ID NO: 190) | |
| | well bc.-F30 | GACAAGTGGCCACAAACCACCAGCACCGGCTTGTGGAAAGGAC | 49 |
| | | GAAACA (SEQ ID NO: 191) | |
| | well bc.-F31 | GACAAGTGGCCACAAACCACCAGCACGATCTTGTGGAAAGGAC | 49 |
| | | GAAACA (SEQ ID NO: 192) | |
| | well bc.-F32 | GACAAGTGGCCACAAACCACCAGCACTCACTTGTGGAAAGGAC | 49 |
| | | GAAACA (SEQ ID NO: 193) | |
| | well bc.-F33 | GACAAGTGGCCACAAACCACCAGCAGGCGCTTGTGGAAAGGA | 49 |
| | | CGAAACA (SEQ ID NO: 194) | |
| | well bc.-F34 | GACAAGTGGCCACAAACCACCAGCATGGCCTTGTGGAAAGGAC | 49 |
| | | GAAACA (SEQ ID NO: 195) | |
| | well bc.-F35 | GACAAGTGGCCACAAACCACCAGCATTTTCTTGTGGAAAGGAC | 49 |
| | | GAAACA (SEQ ID NO: 196) | |
| | well bc.-F36 | GACAAGTGGCCACAAACCACCAGCCAACACTTGTGGAAAGGAC | 49 |
| | | GAAACA (SEQ ID NO: 197) | |
| | well bc.-F37 | GACAAGTGGCCACAAACCACCAGCGGAATCTTGTGGAAAGGAC | 49 |
| | | GAAACA (SEQ ID NO: 198) | |
| | well bc.-F38 | GACAAGTGGCCACAAACCACCAGCTAGCTCTTGTGGAAAGGAC | 49 |
| | | GAAACA (SEQ ID NO: 199) | |
| | well bc.-F39 | GACAAGTGGCCACAAACCACCAGCTATACCTTGTGGAAAGGAC | 49 |
| | | GAAACA (SEQ ID NO: 200) | |
| | well bc.-F40 | GACAAGTGGCCACAAACCACCAGCTCAGACTTGTGGAAAGGAC | 49 |
| | | GAAACA (SEQ ID NO: 201) | |
| | well bc.-F41 | GACAAGTGGCCACAAACCACCAGGACGACCTTGTGGAAAGGA | 49 |
| | | CGAAACA (SEQ ID NO: 202) | |
| | well bc.-F42 | GACAAGTGGCCACAAACCACCAGTAATCGCTTGTGGAAAGGAC | 49 |
| | | GAAACA (SEQ ID NO: 203) | |
| | well bc.-F43 | GACAAGTGGCCACAAACCACCAGTACAGCCTTGTGGAAAGGAC | 49 |
| | | GAAACA (SEQ ID NO: 204) | |
| | well bc.-F44 | GACAAGTGGCCACAAACCACCAGTATAATCTTGTGGAAAGGAC | 49 |
| | | GAAACA (SEQ ID NO: 205) | |
| | well bc.-F45 | GACAAGTGGCCACAAACCACCAGTCATTCCTTGTGGAAAGGAC | 49 |
| | | GAAACA (SEQ ID NO: 206) | |
| | well bc.-F46 | GACAAGTGGCCACAAACCACCAGTCCCGACTTGTGGAAAGGAC | 49 |
| | | GAAACA (SEQ ID NO: 207) | |
| | well bc.-F47 | GACAAGTGGCCACAAACCACCAGTCGAAGCTTGTGGAAAGGAC | 49 |
| | | GAAACA (SEQ ID NO: 208) | |
| | well bc.-F48 | GACAAGTGGCCACAAACCACCAGTCGGCACTTGTGGAAAGGAC | 49 |
| | | GAAACA (SEQ ID NO: 209) | |
| | well bc.-F49 | GACAAGTGGCCACAAACCACCAGGTACTTCTTGTGGAAAGGAC | 49 |
| | | GAAACA (SEQ ID NO: 210) | |
| | well bc.-F50 | GACAAGTGGCCACAAACCACCAGATGGCACTTGTGGAAAGGAC | 49 |
| | | GAAACA (SEQ ID NO: 211) | |
| | well bc.-F51 | GACAAGTGGCCACAAACCACCAGAAGGAGCTTGTGGAAAGGA | 49 |
| | | CGAAACA (SEQ ID NO: 212) | |
| | well bc.-F52 | GACAAGTGGCCACAAACCACCAGCTATCTCTTGTGGAAAGGAC | 49 |
| | | GAAACA (SEQ ID NO: 213) | |
| | well bc.-F53 | GACAAGTGGCCACAAACCACCAGTATATCCTTGTGGAAAGGAC | 49 |
| | | GAAACA (SEQ ID NO: 214) | |
| | well bc.-F54 | GACAAGTGGCCACAAACCACCAGTATTATCTTGTGGAAAGGAC | 49 |
| | | GAAACA (SEQ ID NO: 215) | |
| | well bc.-F55 | GACAAGTGGCCACAAACCACCAGAGCACCCTTGTGGAAAGGAC | 49 |
| | | GAAACA (SEQ ID NO: 216) | |
| | well bc.-F56 | GACAAGTGGCCACAAACCACCAGTCCACACTTGTGGAAAGGAC | 49 |
| | | GAAACA (SEQ ID NO: 217) | |
| | well bc.-F57 | GACAAGTGGCCACAAACCACCAGTTCTCGCTTGTGGAAAGGAC | 49 |
| | | GAAACA (SEQ ID NO: 218) | |
| | well bc.-F58 | GACAAGTGGCCACAAACCACCAGTCCTGCCTTGTGGAAAGGAC | 49 |
| | | GAAACA (SEQ ID NO: 219) | |
| | well bc.-F59 | GACAAGTGGCCACAAACCACCAGCGCGGCCTTGTGGAAAGGAC | 49 |
| | | GAAACA (SEQ ID NO: 220) | |
| | well bc.-F60 | GACAAGTGGCCACAAACCACCAGTGCGCTCTTGTGGAAAGGAC | 49 |
| | | GAAACA (SEQ ID NO: 221) | |
| | well bc.-F61 | GACAAGTGGCCACAAACCACCAGAACGGACTTGTGGAAAGGA | 49 |
| | | CGAAACA (SEQ ID NO: 222) | |
| | well bc.-F62 | GACAAGTGGCCACAAACCACCAGGACGATCTTGTGGAAAGGAC | 49 |
| | | GAAACA (SEQ ID NO: 223) | |
| | well bc.-F63 | GACAAGTGGCCACAAACCACCAGTTGCGGCTTGTGGAAAGGAC | 49 |
| | | GAAACA (SEQ ID NO: 224) | |
| | well bc.-F64 | GACAAGTGGCCACAAACCACCAGCAAGTTCTTGTGGAAAGGAC | 49 |
| | | GAAACA (SEQ ID NO: 225) | |
| | well bc.-F65 | GACAAGTGGCCACAAACCACCAGTACCTTCTTGTGGAAAGGAC | 49 |
| | | GAAACA (SEQ ID NO: 226) | |
| | well bc.-F66 | GACAAGTGGCCACAAACCACCAGGAGCGCCTTGTGGAAAGGA | 49 |
| | | CGAAACA (SEQ ID NO: 227) | |
| | well bc.-F67 | GACAAGTGGCCACAAACCACCAGACGAGGCTTGTGGAAAGGA | 49 |
| | | CGAAACA (SEQ ID NO: 228) | |
| | well bc.-F68 | GACAAGTGGCCACAAACCACCAGAATCGTCTTGTGGAAAGGAC | 49 |
| | | GAAACA (SEQ ID NO: 229) | |
| | well bc.-F69 | GACAAGTGGCCACAAACCACCAGGAGTCTCTTGTGGAAAGGAC | 49 |
| | | GAAACA (SEQ ID NO: 230) | |
| | well bc.-F70 | GACAAGTGGCCACAAACCACCAGAGACCACTTGTGGAAAGGA | 49 |
| | | CGAAACA (SEQ ID NO: 231) | |
| | well bc.-F71 | GACAAGTGGCCACAAACCACCAGAATTCCCTTGTGGAAAGGAC | 49 |
| | | GAAACA (SEQ ID NO: 232) | |
| | well bc.-F72 | GACAAGTGGCCACAAACCACCAGAGATGTCTTGTGGAAAGGAC | 49 |
| | | GAAACA (SEQ ID NO: 233) | |
| | well bc.-F73 | GACAAGTGGCCACAAACCACCAGGGAGAACTTGTGGAAAGGA | 49 |
| | | CGAAACA (SEQ ID NO: 234) | |
| | well bc.-F74 | GACAAGTGGCCACAAACCACCAGATAGATCTTGTGGAAAGGAC | 49 |
| | | GAAACA (SEQ ID NO: 235) | |
| | well bc.-F75 | GACAAGTGGCCACAAACCACCAGTATGCACTTGTGGAAAGGAC | 49 |
| | | GAAACA (SEQ ID NO: 236) | |
| | well bc.-F76 | GACAAGTGGCCACAAACCACCAGCCTATTCTTGTGGAAAGGAC | 49 |
| | | GAAACA (SEQ ID NO: 237) | |
| | well bc.-F77 | GACAAGTGGCCACAAACCACCAGTAACCGCTTGTGGAAAGGAC | 49 |
| | | GAAACA (SEQ ID NO: 238) | |
| | well bc.-F78 | GACAAGTGGCCACAAACCACCAGAGTCTCCTTGTGGAAAGGAC | 49 |
| | | GAAACA (SEQ ID NO: 239) | |
| | well bc.-F79 | GACAAGTGGCCACAAACCACCAGTGGAGCCTTGTGGAAAGGAC | 49 |
| | | GAAACA (SEQ ID NO: 240) | |
| | well bc.-F80 | GACAAGTGGCCACAAACCACCAGCTGCATCTTGTGGAAAGGAC | 49 |
| | | GAAACA (SEQ ID NO: 241) | |
| | well bc.-F81 | GACAAGTGGCCACAAACCACCAGCAGGCCCTTGTGGAAAGGAC | 49 |
| | | GAAACA (SEQ ID NO: 242) | |
| | well bc.-F82 | GACAAGTGGCCACAAACCACCAGCAGTAACTTGTGGAAAGGAC | 49 |
| | | GAAACA (SEQ ID NO: 243) | |
| | well bc.-F83 | GACAAGTGGCCACAAACCACCAGGTTACCCTTGTGGAAAGGAC | 49 |
| | | GAAACA (SEQ ID NO: 244) | |
| | well bc.-F84 | GACAAGTGGCCACAAACCACCAGACTGCTCTTGTGGAAAGGAC | 49 |
| | | GAAACA (SEQ ID NO: 245) | |
| | well bc.-F85 | GACAAGTGGCCACAAACCACCAGCTAAGGCTTGTGGAAAGGAC | 49 |
| | | GAAACA (SEQ ID NO: 246) | |
| | well bc.-F86 | GACAAGTGGCCACAAACCACCAGATCCAACTTGTGGAAAGGAC | 49 |
| | | GAAACA (SEQ ID NO: 247) | |
| | well bc.-F87 | GACAAGTGGCCACAAACCACCAGGTGAGACTTGTGGAAAGGA | 49 |
| | | CGAAACA (SEQ ID NO: 248) | |
| | well bc.-F88 | GACAAGTGGCCACAAACCACCAGTAAGACCTTGTGGAAAGGAC | 49 |
| | | GAAACA (SEQ ID NO: 249) | |
| | well bc.-F89 | GACAAGTGGCCACAAACCACCAGCGTTAGCTTGTGGAAAGGAC | 49 |
| | | GAAACA (SEQ ID NO: 250) | |
| | well bc.-F90 | GACAAGTGGCCACAAACCACCAGGTGTATCTTGTGGAAAGGAC | 49 |
| | | GAAACA (SEQ ID NO: 251) | |
| | well bc.-F91 | GACAAGTGGCCACAAACCACCAGGAATGCCTTGTGGAAAGGAC | 49 |
| | | GAAACA (SEQ ID NO: 252) | |
| | well bc.-F92 | GACAAGTGGCCACAAACCACCAGAGAGTACTTGTGGAAAGGA | 49 |
| | | CGAAACA (SEQ ID NO: 253) | |
| | well bc.-F93 | GACAAGTGGCCACAAACCACCAGAGTCCACTTGTGGAAAGGAC | 49 |
| | | GAAACA (SEQ ID NO: 254) | |
| | well bc.-F94 | GACAAGTGGCCACAAACCACCAGCTATGGCTTGTGGAAAGGAC | 49 |
| | | GAAACA (SEQ ID NO: 255) | |
| | well bc.-F95 | GACAAGTGGCCACAAACCACCAGACCTTACTTGTGGAAAGGAC | 49 |
| | | GAAACA (SEQ ID NO: 256) | |
| | well bc.-F96 | GACAAGTGGCCACAAACCACCAGCGCCAACTTGTGGAAAGGAC | 49 |
| | | GAAACA (SEQ ID NO: 257) | |
| Reverse | well bc.-R | TCTAAAGCGCATGCTCCAGAC (SEQ ID NO: 258) | 21 |
| primer | | | |

TABLE 6
Three-step PCR (step 2 primers)

| PCR | Forward | | Length |
|---|---|---|---|
| reaction | primer | PS-F whole sequence | (bp) |
| 1 | PS-F1 | ACACTCTTTCCCTACACGACGCTGTTCCGATCTTCGCATAGACAAG | 63 |
| | | TGGCCACAAACCACCAG (SEQ ID NO: 259) | |
| | PS-R1 | GTGACTGGAGTTCAGACGTGTGCTCTTCCGATCGTCTAAAGCGCAT | 55 |
| | | GCTCCAGAC (SEQ ID NO: 260) | |
| 2 | PS-F2 | ACACTCTTTCCCTACACGACGCTCTTCCGATCTCGCATAGACAAGT | 62 |
| | | GGCCACAAACCACCAG (SEQ ID NO: 261) | |
| | PS-R2 | GTGACTGGAGTTCAGACGTGTGCTCTTCCGATCTGTCTAAAGCGCA | 56 |
| | | TGCTCCAGAC (SEQ ID NO: 262) | |
| 3 | PS-F3 | ACACTCTTTCCCTACACGACGCTCTTCCGATCTGCATAGACAAGTG | 61 |
| | | GCCACAAACCACCAG (SEQ ID NO: 263) | |
| | PS-R3 | GTGACTGGAGTTCAGACGTGTGCTCTTCCGATCCTGTCTAAAGCGC | 57 |
| | | ATGCTCCAGAC (SEQ ID NO: 264) | |
| 4 | PS-F4 | ACACTCTTTCCCTACACGACGCTCTTCCGATCTCATAGACAAGTGG | 60 |
| | | CCACAAACCACCAG (SEQ ID NO: 265) | |
| | PS-R4 | GTGACTGGAGTTCAGACGTGTGCTCTTCCGATCACTGTCTAAAGCG | 58 |
| | | CATGCTCCAGAC (SEQ ID NO: 266) | |
| 5 | PS-F5 | ACACTCTTTCCCTACACGACGCTCTTCCGATCTATAGACAAGTGGC | 59 |
| | | CACAAACCACCAG (SEQ ID NO: 267) | |
| | PS-R5 | GTGACTGGAGTTCAGACGTGTGCTCTTCCGATCCACTGTCTAAAGC | 59 |
| | | GCATGCTCCAGAC (SEQ ID NO: 268) | |
| 6 | PS-F6 | ACACTCTTTCCCTACACGACGCTCTTCCGATCTTAGACAAGTGGCC | 58 |
| | | ACAAACCACCAG (SEQ ID NO: 269) | |
| | PS-R6 | GTGACTGGAGTTCAGACGTGTGCTCTTCCGATCTCACTGTCTAAAG | 60 |
| | | CGCATGCTCCAGAC (SEQ ID NO: 270) | |
| 7 | PS-F7 | ACACTCTTTCCCTACACGACGCTCTTCCGATCTAGACAAGTGGCCA | 57 |
| | | CAAACCACCAG (SEQ ID NO: 271) | |
| | PS-R7 | GTGACTGGAGTTCAGACGTGTGCTCTTCCGATCATCACTGTCTAAA | 61 |
| | | GCGCATGCTCCAGAC (SEQ ID NO: 272) | |
| 8 | PS-F8 | ACACTCTTTCCCTACACGACGCTCTTCCGATCTGACAAGTGGCCAC | 56 |
| | | AAACCACCAG (SEQ ID NO: 273) | |
| | PS-R8 | GTGACTGGAGTTCAGACGTGTGCTCTTCCGATCGATCACTGTCTAA | 62 |
| | | AGCGCATGCTCCAGAC (SEQ ID NO: 274) | |

### Sequencing Data Analysis.

For extraction and quantification of well barcode (well bc), antibody-specific barcode (Ab bc) and UMI, we devised a customed pipeline using a combination of UMI-tools and unix commands. This pipeline efficiently quantifies barcode and UMI combinations, while accounting for potential sequencing errors.

First, the “UMI-tools whitelist” utility was used to generate a whitelist of barcode and UMI combinations from the position 1-6, 21-26, 27-41 of the fastq reads. The whitelist was then washed with respect to a ground truth barcode list to reduce false-positive rates of the read quantification. Then the “UMI-tools extract” utility was used to extract barcode and UMI, while base pair corrections were performed on barcode-UMI pairs 2 Hamming Distance from the whitelist barcode-UMI pairs. Finally, unix commands were used to demultiplex the well barcode, antibody-specific barcode, and UMI to acquire the final count table that stores the barcode-UMI quantification.

### EXAMPLES

### Example 1. Design of MaMBA

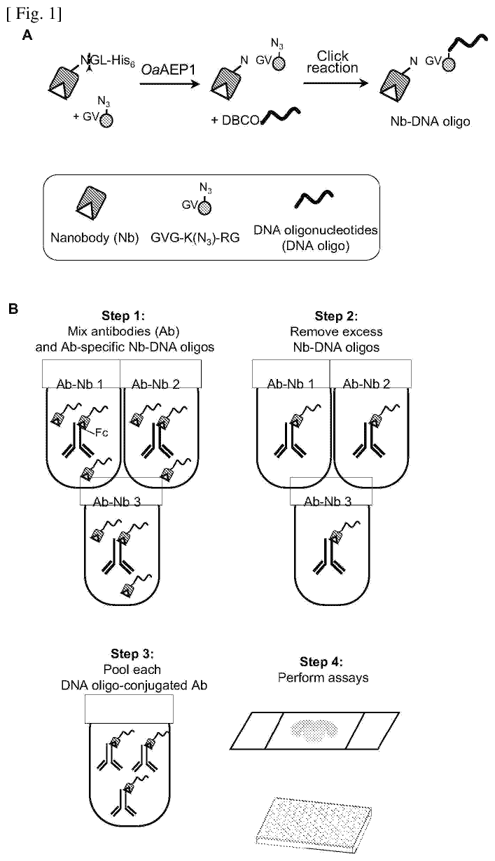

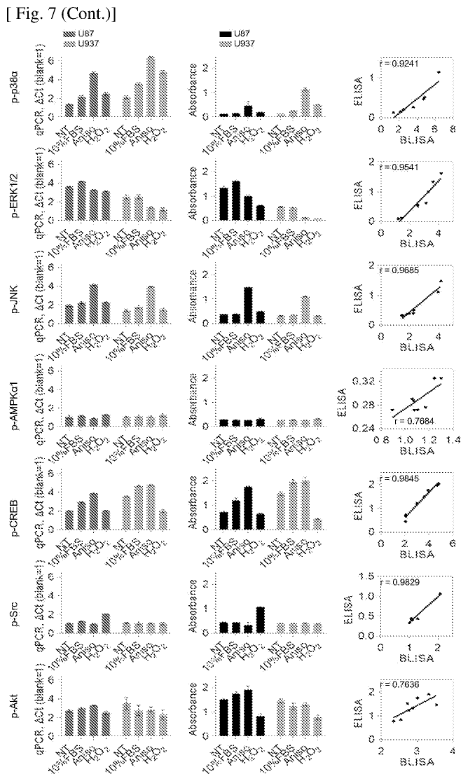

As the adaptors, nanobodies targeting the fragment crystallizable (Fc) region of IgGs are recombinantly expressed, fused with Asn-Gly-Leu (NGL) tripeptide recognition motif and 6×His tag (for protein purification) at the C-termini. To functionalize nanobodies, an enzymatic reaction is utilized which the NGL motifs of nanobodies are recognized by the protein ligase, a recombinant Oldenlandia affinis asparaginyl endopeptidase (OaAEP1). OaAEP1 cuts the NGL motif, ligates the GV-based azide-modified peptide, GVGK (N₃) RG, and yields nanobody-NGVGK (N₃) RG products which are poorly recognized by OaAEP1, making it efficient for the production. Then the azide-functionalized nanobodies are conjugated with DBCO-modified (azide-reactive) DNA oligonucleotides (DNA oligos) via the click reaction (FIG. 8A and FIG. 1A). The DNA oligo-conjugated nanobodies (Nb-DNA oligos) are ready for barcoding antibodies to execute multiplexed detection.

For antibodies barcoding, each antibody is mixed with an antibody-specific Nb-DNA oligo (specific DNA sequence). DNA oligos are conjugated to the Fc domain of antibodies through the site-specific binding of nanobodies to form the DNA-barcoded complex (Ab-Nb1 to Ab-Nbn), and the excess Nb-DNA oligos are removed by filtration. Then each DNA oligo-conjugated antibody could be directly pooled and applied to different assay such as immunostaining, quantitative protein detection and the like (FIG. 1B).

### Example 2. MaMBA Enables Multiplexed In Situ Protein Imaging by misHCR

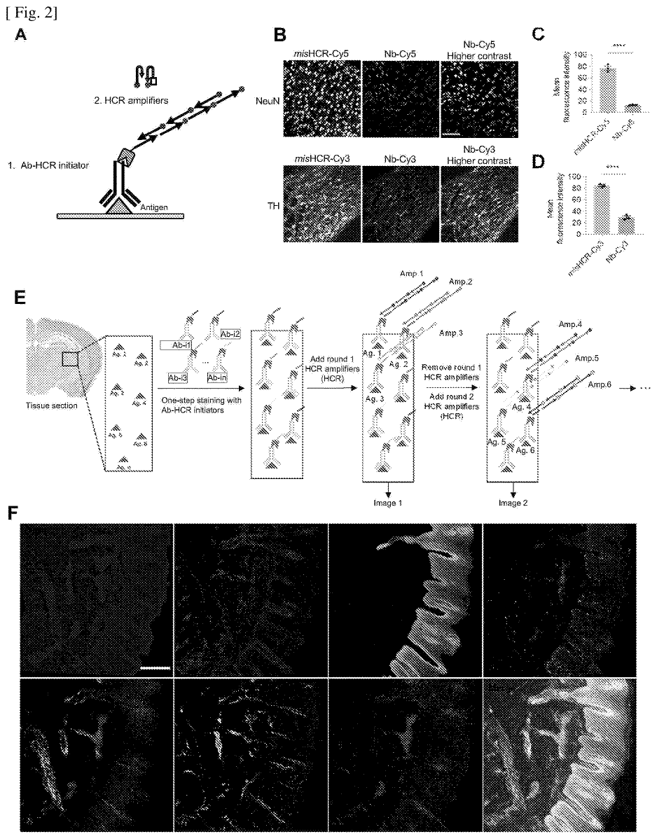

Firstly, we applied MaMBA to immunostaining for multiplexed in situ protein imaging. Previously, we developed isHCR, a DNA-based method for amplification of immunofluorescence signals, which utilizes iterative DNA hairpins opening by a pair of fluorophore-modified DNA hairpins (HCR amplifiers) triggered by an initiating DNA strand (HCR initiator) bound to the epitope (the antigen target of antibody) through multiple-step binding. However, the redundant protocol makes it laborious and time-consuming to anchor the HCR initiator to the epitope, and the secondary antibodies staining strategy limits the multiplexity. After barcoding antibodies with different orthogonal HCR initiators (Ab-HCR initiators), we could easily minimize HCR initiators anchoring procedure to only one step, and expand the multiplexity utilizing the power of DNA (FIG. 2A).

To examine the performance of misHCR, we immunostained mouse brain sections with antibodies against NeuN or tyrosine hydroxylase (Th), bound with equal amounts of HCR initiator-conjugated nanobodies and fluorophore-conjugated nanobodies. Compared with fluorophore-conjugated nanobodies (Nb-Cy5/Cy3), HCR initiator-conjugated nanobodies following the signal amplification (misHCR-Cy5/Cy3) showed dramatical signal increase, and the target specificity was confirmed by the same pattern displayed in the higher contrast images of Nb-Cy5/Cy3 staining (FIGS. 2B-2D). We also confirmed that misHCR did not affect the spatial resolution in diffraction-limited confocal imaging as performed by the traditional method (FIGS. 8B-8D).

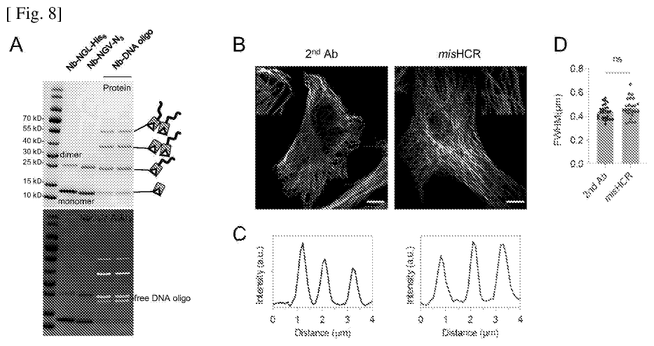

Next, we performed misHCR for multiplexed immunostaining, and imaged through several rounds of HCR and washing (using formamide to dehybridize HCR amplifiers). Based on the HCR initiator-barcoded antibodies by MaMBA, we can do one-step antibodies incubation independent of the host species of the antibodies, and then image sequentially for multiple epitopes by several rounds of hybridization and dehybridization of HCR amplifiers (FIG. 2E). We compared the pre- and post-washing (dehybridization of HCR amplifiers) images between image rounds, showing that signals were efficiently removed after HCR amplifiers removal (FIG. 9A). We also evaluated the strand-binding integrity of misHCR for antibodies against NeuN, NF-H and Th by repetitively going through five times of HCR-and-washing cycle. It showed highly reproducible localization patterns of the signal at each time of HCR, and we found that over 80% of the signal was recovered even after four times of HCR amplifiers washing (FIGS. 9C and 9D).

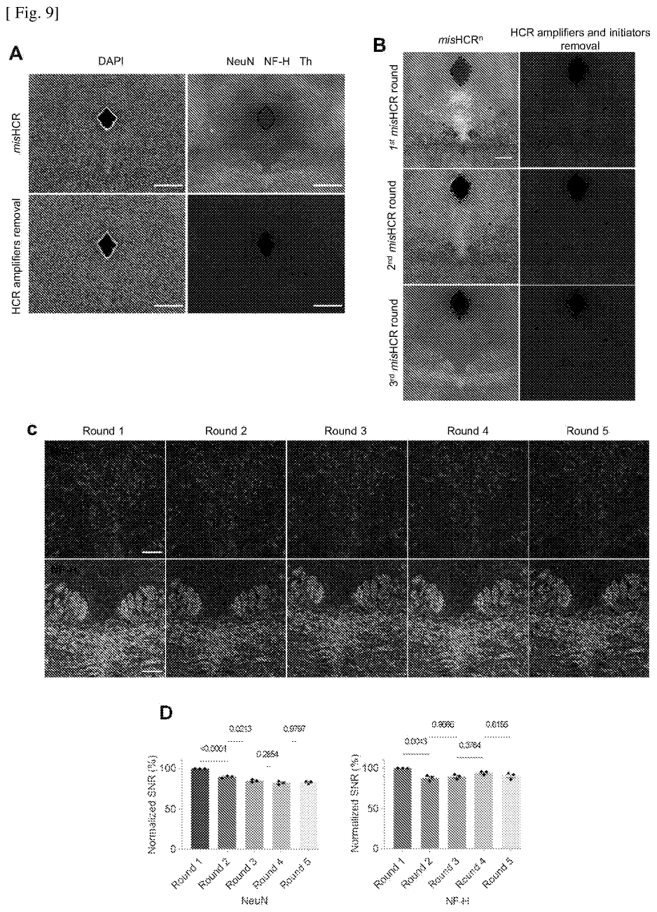

Next, we applied misHCR on healthy and psoriatic human skin sections against six markers, including platelet-derived growth factor receptor A (PDGFRa) as a progenitor marker for mesenchymal cells, keratin 14 (KRT14) as a maker for keratinocytes in the epidermis, dopachrome tautomerase (DCT) as a marker for melanoblasts, CD31 as a marker for endothelial cells, alpha smooth muscle actin (aSMA) as a marker for myofibroblasts, and CD45 as a marker for hematopoietic cells except erythrocytes and platelets, that consists of four rabbit (Rb) IgG antibodies and two mouse (Mus) IgG1 antibodies (FIG. 10 and FIG. 2F). Each showed defined imaging patterns in both healthy and psoriatic skins. The psoriatic skin displayed dilated blood vessels, accumulation of white blood cells, and abnormal proliferation of keratinocytes compared with the healthy skin, which was consistent with previous reports. Thus, misHCR utilizing MaMBA for antibodies barcoding with orthogonal HCR initiators enables specific and multiplexed in situ protein imaging in a streamlined workflow.

### Example 3. Cleavable MaMBA Enables Increased Multiplexity with Multi-Round misHCR (misHCRⁿ)

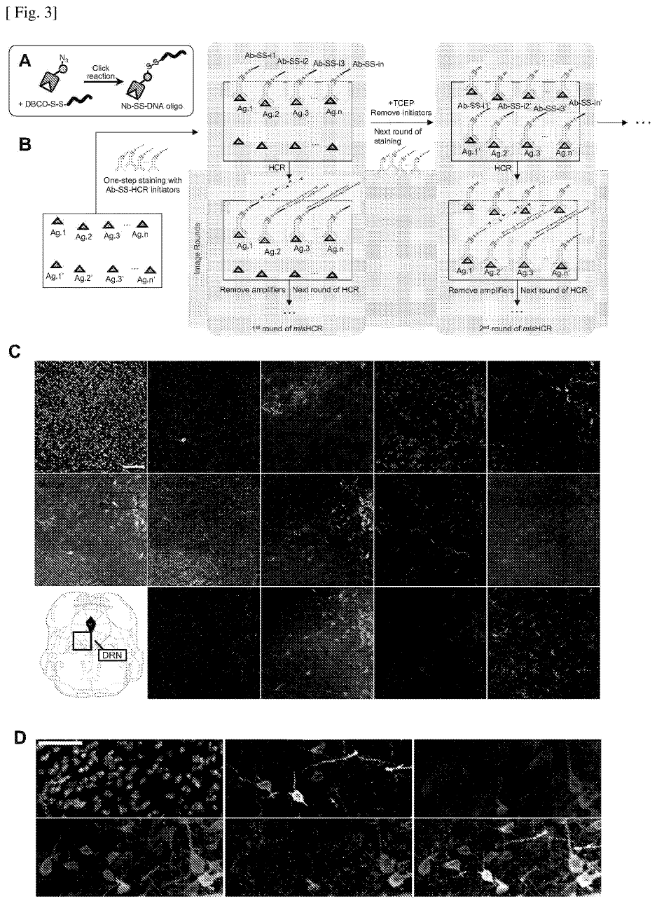

Considering the challenge of designing orthogonal DNA hairpins, it is unrealistic to produce abundant sets of HCR amplifiers to satisfy the needs of higher imaging throughput. Furthermore, antibodies stripping technique is also challenging due to the risk of cross-reactivity and impaired epitope integrity. Thus, direct removal of HCR initiators from antibodies would be a feasible way. To obtain enhanced multiplexity and improve imaging throughput, we developed a cleavable MaMBA that the azide-modified nanobodies are conjugated with DNA oligos bearing a disulfide linker and DBCO. The disulfide linker could be cleaved by reductant (FIG. 3A).

After one imaging round of misHCR, HCR initiators could be released via reductive cleavage and washed away. Then the next round of misHCR could be performed and re-employed the same set of HCR initiators (FIG. 3B). We compared the pre- and post-washing images between misHCR rounds, and signals were efficiently removed by removing the HCR initiators (FIG. 9B).

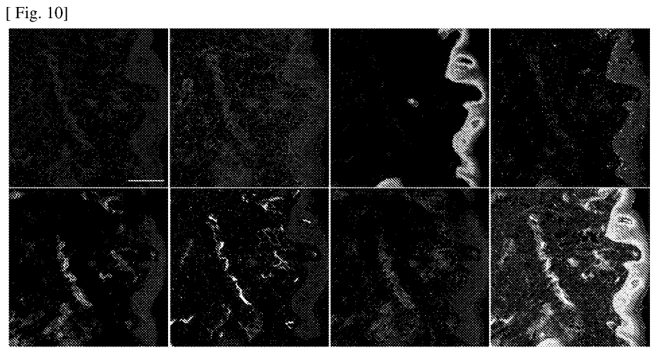

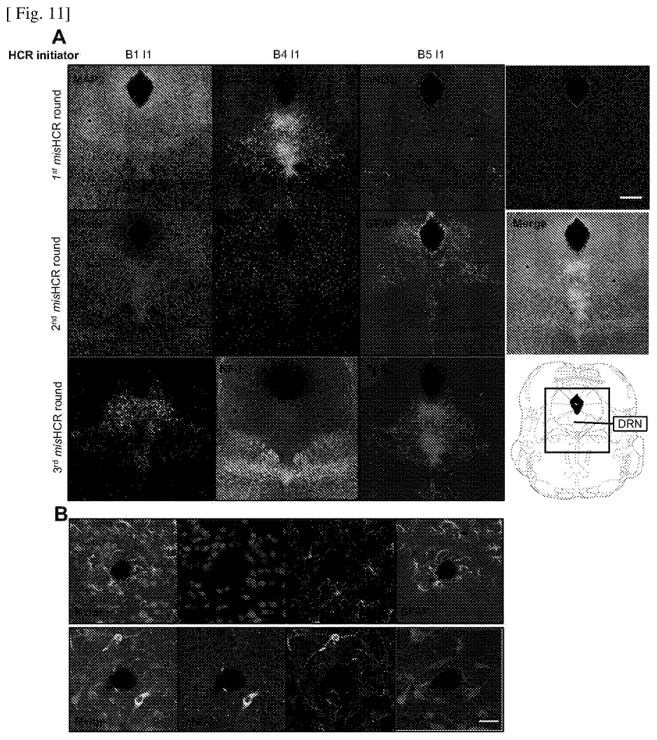

We then validated the multi-round misHCR (misHCRⁿ) in 35-μm-thick mouse brain cryosections and imaged nine targets by three rounds of misHCR that repetitively employed three orthogonal HCR initiators (B1 I1, B4 I1 and B5 I1) (FIG. 11A). We selected antibodies, all from Rb host species, against several targets that have defined imaging distribution patterns such as NeuN, glial fibrillary acidic protein (GFAP), neurofilament-H (NF-H), tryptophan hydroxylase 2 (Tph2), and ionized calcium binding adaptor molecule 1 (Iba1). We validated the specificity of the barcoded antibodies by comparing the staining colocalization between misHCR and conventional indirect immunofluorescence (FIG. 12). We also compared the staining patterns of different cell-type markers (NeuN for neuron, Iba1 for microglia, and GFAP for astrocyte) as well as different neuronal markers (nNOS for nNOS-expressing neuron, Th for dopaminergic neuron, and Tph2 for serotonergic neuron), and we found no cross-reaction among the barcoded antibodies for intra- or inter-misHCR round (FIG. 11B).

We further demonstrated a twelve-target protein imaging in dorsal raphe nucleus (DRN) of mouse brain by two rounds of misHCR, employing nine orthogonal HCR initiators conjugated to antibodies that were all from Rb host species (FIG. 3C). Through comparison of the staining patterns, we observed three types of DDC-expressing neurons in DRN, including dopaminergic neuron (DDC⁺Th⁺Tph2⁻5-HT⁻ cell), serotonergic neuron (DDC⁺Th⁻Tph2⁺5-HT⁺ cell), and so-called D-neuron (DDC⁺Th⁻Tph2⁻5-HT⁻ cell) (FIG. 3D). Thus, misHCRⁿ increases the imaging throughput by performing multi-round misHCR, which efficiently recycles the HCR initiators by utilizing cleavable MaMBA to easily remove anchored HCR initiators via reductive cleavage.

### Example 4. Expanding Cleavable MaMBA to Multiplexed Quantitative Protein Detection by BLISA

To further demonstrate the flexibility and diversity of MaMBA application, we adapted cleavable MaMBA to ELISA-like assay and developed BLISA for multiplexed quantitative protein detection. Taking advantages of the DNA-based barcoding property and its site-specific conjugation to antibody, BLISA enables detection and quantification of multiple target proteins at the same time. DNA barcodes are released by reductive cleavage, which decreases the interference from other DNA sticked or non-specifically bound to the immobilized surface, and then quantified by qPCR or sequencing technique.

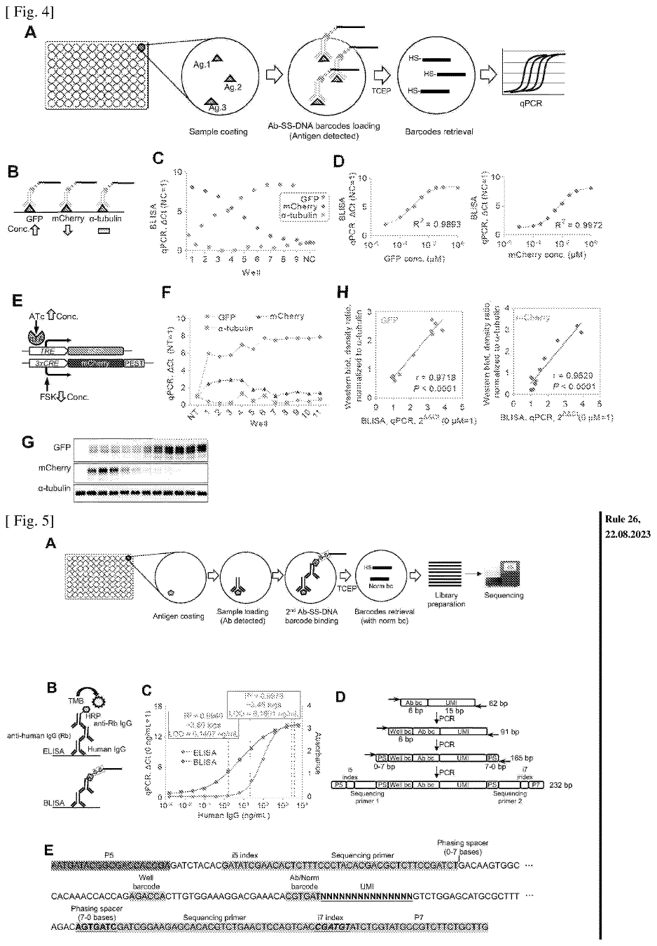

We first verified BLISA in cell lysates by directly detecting samples immobilized on microplate surfaces. DNA-barcoded antibodies are then loaded to detect their targets on the plate. After washing away the unbound antibodies, the DNA barcodes are retrieved by reductant (e.g. TCEP) and quantified by qPCR (FIG. 4A).

Considering the background of BLISA created not only from non-specific antibody binding but also from non-specific binding of the DNA conjugates, we used protein-free blocking buffer (PFBB) and added sheared Salmon sperm DNA (ssDNA) in antibody incubation solution to block the unwanted binding of antibodies and DNA barcodes. To assess whether BLISA could distinguish different target proteins in a single well, we immobilized purified GFP and mCherry with known concentration gradients on the microplate, mixed with 1 μg/mL wild-type cell lysates (to mimic the cellular matrix environment), and then detected three targets including GFP, mCherry, and α-tubulin (from cell lysates) (FIG. 4B). Both GFP and mCherry detected by BLISA correlated well with their designed gradient (R²=0.9893 and 0.9972, respectively) without significant disturbance by each other (FIGS. 4C and 4D).

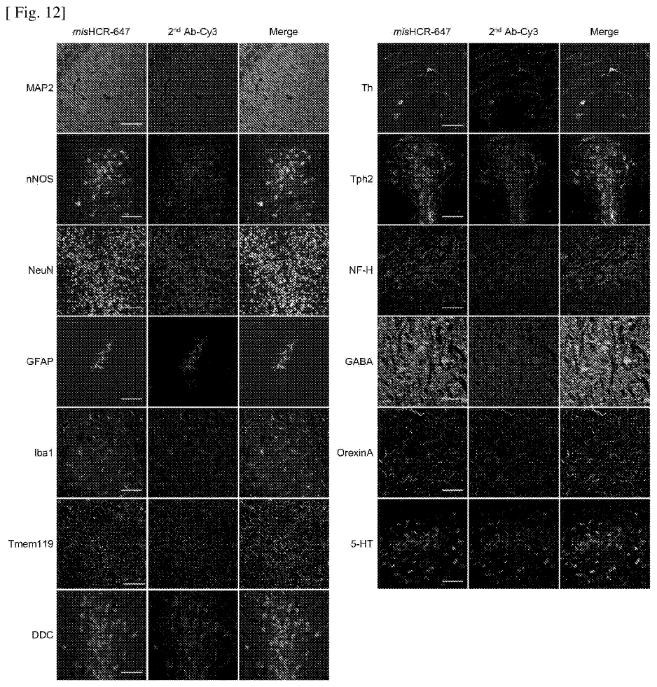

We also applied BLISA for endogenously expressed proteins in cells by transient transfection of target genes leaded by drug-inducible promoters. We introduced the Tet-ON system (TRE promoter triggered by drug-bound rtTA transcription factor) for GFP expression controlled by anhydrotetracycline hydrochloride (ATc, a tetracycline derivative), and c-AMP response element (3×CRE) for mCherry expression induced by forskolin (FSK) (FIG. 4E, FIGS. 13A and 13C). We then detected GFP, mCherry and α-tubulin (as a loading control for normalization) simultaneously by BLISA, meanwhile compared with Western blot, which acted as our ‘gold standard’ to measure the relative expression levels of GFP and mCherry with a series of triggers by ATc and FSK. We expressed these two drug-triggered systems separately in HEK293T cells. The results of BLISA (α-tubulin-normalized ΔCt) and Western blot (α-tubulin-normalized density ratio) were highly correlated (r=0.9724, P<0.0001 for GFP; r=0.9647, P<0.0001 for mCherry) (FIGS. 13B and 13D). Then we combined them by simultaneous transfection in HEK293T cells, and it showed that the results of the 3-plex BLISA detection were still highly correlated with that of Western blot (r=0.9718, P<0.0001 for GFP and r=0.9520, P<0.0001 for mCherry) (FIGS. 4F-4H). Therefore, taking advantage of the feasibility of MaMBA, BLISA was capable of executing multiplexed quantitative protein detection assay.

To combine BLISA with high-throughput sequencing, the DNA barcode was designed to contain a 6-bp antibody-dedicated barcode (Ab-bc) and a 15-bp unique molecular identifier (UMI). Retrieved DNA barcodes are added by a well-specific barcode (well-bc) through PCR amplification. Then each well-specific DNA barcodes are pooled to prepare indexed library for sequencing (FIG. 13E). Each target proteins of each well are identified by their antibody barcode and well barcode, and quantified by UMI counts. We assessed the sequencing readout of BLISA for the samples as designed in FIG. 4C, and validated the dose-dependent curves of GFP and mCherry (R²=0.9827 and 0.9843, respectively) (FIGS. 13F and 13G), demonstrating the availability of sequencing-combined BLISA.

### Example 5. Sequencing-Combined BLISA for Anti-SARS-CoV-2 Spike RBD IgG Detection in Human Serum

To investigate whether BLISA combined with high-throughput sequencing could accurately detect proteins level in human samples, we first performed BLISA for anti-severe acute respiratory syndrome coronavirus 2 (SARS-CoV-2) Spike receptor-binding-domain (RBD) IgG detection in human serum. SARS-CoV-2 uses Spike RBD to bind to the cell receptor ACE2 and triggers the viral infection into the cell. Thus, a high-throughput serological test for antibodies against SARS-CoV-2 Spike RBD is an essential component for estimating SARS-CoV-2 vaccine efficacy, especially in the vulnerable and high-risk population subgroups such as healthcare workers.

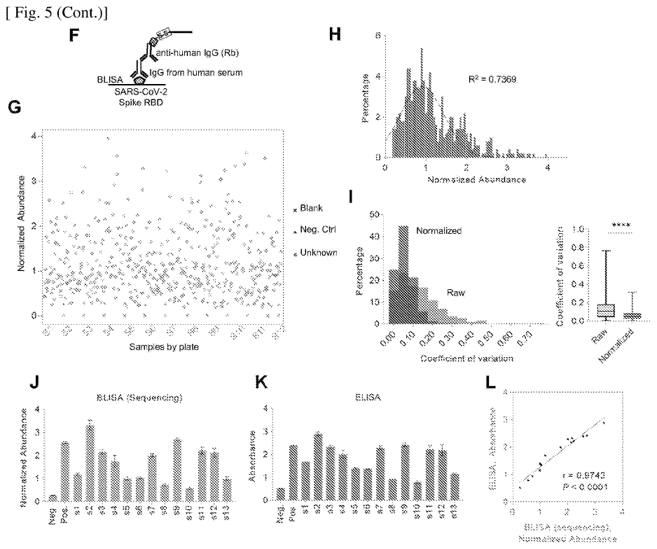

To achieve higher sensitivity, we applied sandwich-based immunoassay in which the analytes (the anti-SARS-CoV-2 Spike RBD IgGs in serum) are captured by the immobilized antigens (the purified SARS-CoV-2 Spike RBD proteins) and detected by the DNA-barcoded secondary (2^{nd}) antibodies (the anti-human IgG antibodies). DNA barcodes are retrieved by TCEP with a known amount of normalization barcodes (norm bc), following library preparation for sequencing (FIGS. 5A and 5F).

To assess the sensitivity of BLISA, we compared the human IgG detection curves between BLISA and ELISA. For BLISA, known amounts of human IgGs are immobilized on microplate and detected by DNA-barcoded anti-human IgG antibodies following qPCR readouts. For ELISA, immobilized human IgGs are detected by the anti-human IgG antibodies (from Rb host species) which are detected by horseradish peroxidase (HRP)-conjugated anti-Rb IgG antibodies following colorimetric readouts with 3,3′,5,5′-Tetramethylbenzidine (TMB) substrates (FIG. 5B). The results showed ~44-fold higher sensitivity and ~0.15 logs wider dynamic range in BLISA, as compared to ELISA (FIG. 5C).

As the library preparation for sequencing, the retrieved DNA barcodes and normalization barcodes were further barcoded by three-step PCR amplifications that sequentially adding well bc, phasing spacers and sequencing adaptors (FIGS. 5D and 5E). In phasing spacers addition, well-barcoded DNA were pooled and separated to 8 PCR reactions for adding various numbers of bases as spacers to both ends with 7 bases in total (0 base and 7 bases, 1 base and 6 bases, 2 bases and 5 bases, etc.), which allows higher input for the amplicon sequencing by enriching the base diversity. We performed the sequencing-combined BLISA against anti-SARS-CoV-2 Spike RBD IgG with a throughput of over 1000 wells in 12 microplates (S1 to S12) for ~500 human serum samples in duplicates (FIG. 5G). Results from the sequencing data fitted a Gaussian distribution (R²=0.7369) (FIG. 5H).

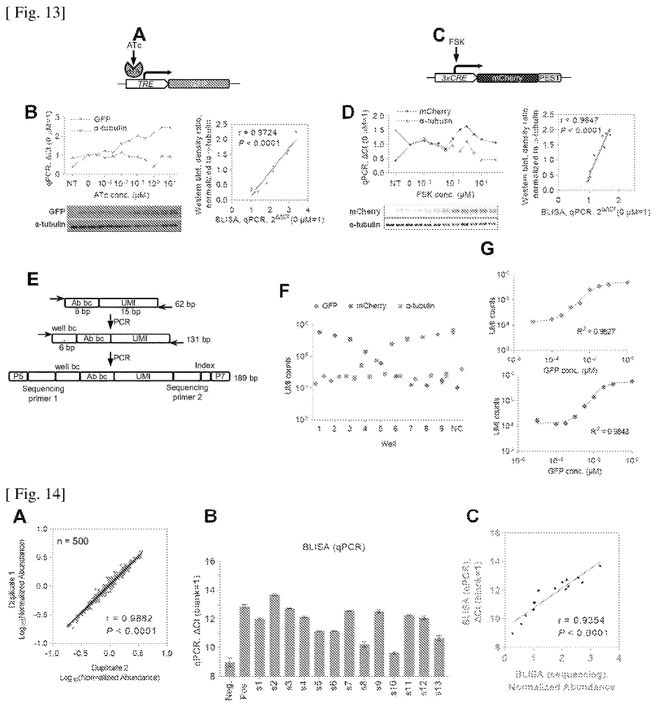

For displaying the IgG abundance, data were normalized by dividing IgG's UMI counts by UMI counts of normalization barcodes. We found that the variability of the signals was dramatically reduced after normalization, with 98.4% of CV lower than 0.2 and high correlation between the duplicates (r=0.9882, P<0.0001), demonstrating the reproductivity of sequencing-combined BLISA with the participation of normalization barcodes (FIGS. 5I, and FIG. 14A). Meanwhile, we randomly selected a portion of the serum (n=13; s1 to s13) and positioned them to different plates for sequencing. Results from two BLISA outputs (qPCR and sequencing) were consistent (r=0.9354, P<0.0001), and the IgG abundances displayed by sequencing were excellently correlated with the ELISA results (r=0.9743, P<0.0001), showing the high accuracy and low plate-to-plate variability of BLISA (FIGS. 5J-L and FIGS. 14B and 14C).

### Example 6. 2-Plex Sequencing-Combined BLISA for Detecting Hepatitis B Virus (HBV) Antigens in Human Serum

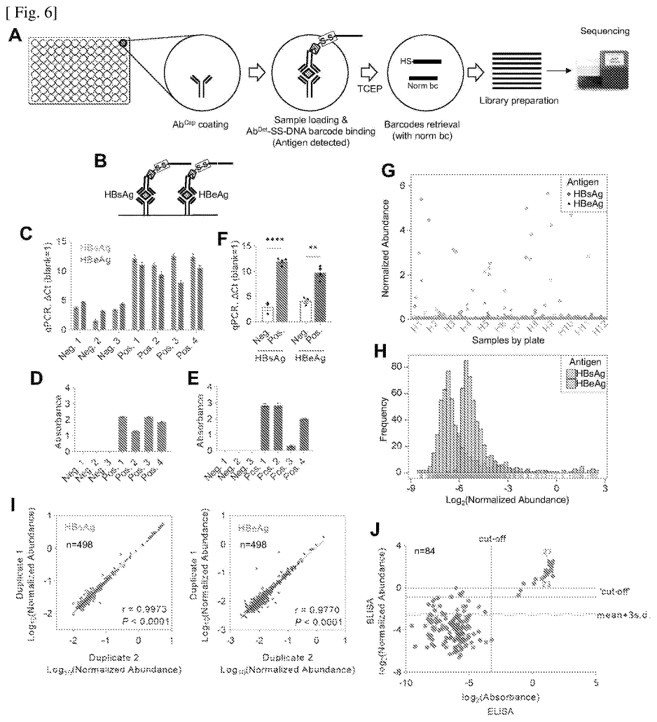

To further examine whether sequencing-combined BLISA is compatible to simultaneously detect more than one target in large-scale human samples, we performed 2-plex BLISA for detecting hepatitis B surface antigen (HBsAg) and hepatitis B e antigen (HBeAg) in human serum which were essential antigen markers for hepatitis B diagnosis. While anti-SARS-CoV-2 Spike RBD IgG detection used an IgG-detected sandwich strategy, HBsAg and HBeAg detection employed an antigen-detected sandwich strategy using a pair of antibodies, namely, capture antibodies (Ab^{Cap}) and detection antibodies (Ab^{Det}) (FIGS. 6A and 6B).

Analytes (HBsAg or HBeAg) are captured by the immobilized Ab^{Cap} and detected by the DNA-barcoded Ab^{Det}, which are distinguished by the antibody-dedicated barcodes. We firstly verified 2-plex BLISA via qPCR in double-negative and double-positive human serum samples that had been defined by commercial ELISA kits (FIGS. 6C-6E). And it showed significant distinction between negative and positive samples in both HBsAg and HBeAg detection (FIG. 6F). We next performed the 2-plex sequencing-combined BLISA against HBsAg and HBeAg with a throughput of over 1000 wells in 12 microplates (H1 to H12) for ~500 human serum samples in duplicates (FIGS. 6G and 6H). Results between duplicates in both HBsAg and HBeAg groups were highly correlated (r=0.9973, P<0.0001 for HBsAg; r=0.9770, P<0.0001 for HBeAg) (FIG. 61). With the abundances over the total average either in HBsAg or HBeAg group, we verified 84 samples by ELISA and found 22 HBsAg⁺HBeAg⁻ samples and 1 HBsAg⁺HBeAg⁺ samples (#23), correlated with the BLISA results (HBsAg, r=0.9517, P<0.0001; HBeAg, r=0.7390, P<0.0001) (FIG. 6J).

### Example 7. Multiplexed Phospho-Proteins Detection in Cell Lysates by BLISA Based on Magnetic Beads

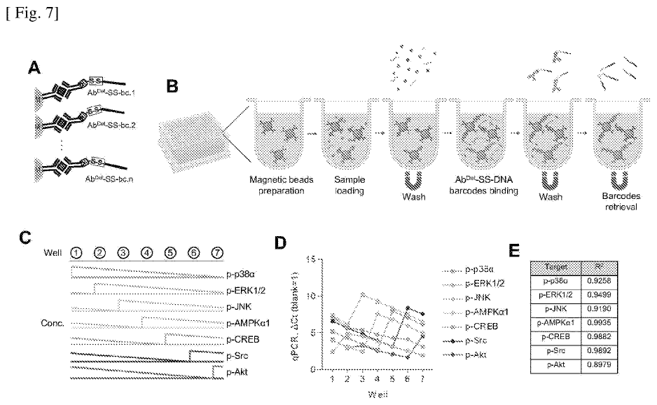

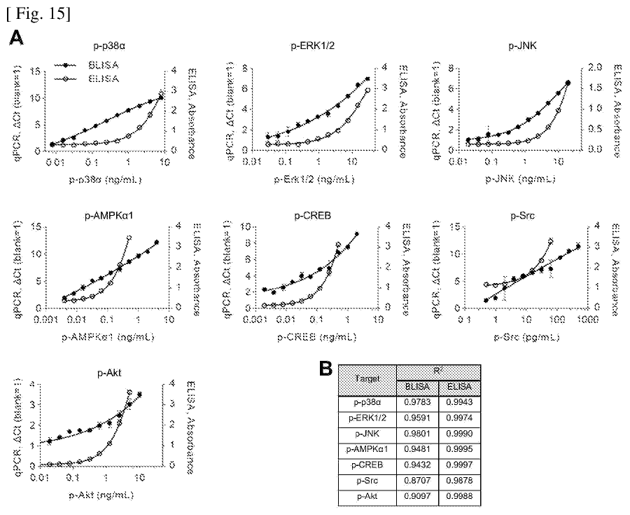

To enhance the multiplexity of BLISA, we switched to the magnetic beads (MB) as the assay binding surface. We performed sandwich-based immunoassay in which capture antibodies were bound to the Epoxy-modified MB, and antigens were identified by their unique DNA barcodes retrieved from the detection antibodies (FIG. 7A). Firstly, MB conjugated with capture antibodies for each target proteins are mixed and aliquoted into each wells of the deep-well plate. Samples were loaded, and the target proteins were captured, followed by washing steps to remove the unbound biomolecules. Then, the target proteins were detected by the Ab^{Det}-SS-DNA barcodes mixtures. After washing away the excess Ab^{Det}-SS-DNA barcodes, DNA barcodes were retrieved by TCEP and quantified (FIG. 7B). We confirmed the standard curves of seven phospho-proteins in BLISA, which indicated higher sensitivity of BLISA compared with ELISA (FIG. 15). To verified the multiplexing of MB-based BLISA, we simultaneously detected the seven purified phospho-proteins (p-p38α, p-ERK1/2, p-JNK, p-AMPKα1, p-CREB, p-Src, and p-Akt), which were loaded within seven sample wells in different concentration gradient frames (FIG. 7C). All phospho-proteins detected by MB-based BLISA highly correlated with their designed gradient frames (R²=0.9258, 0.9499, 0.9190, 0.9935, 0.9882, 0.9892, and 0.8979, respectively) without significant disturbance by each other (FIGS. 7D and 7E). We next performed the 7-plex BLISA for the phospho-proteins in cell lysates of U87 (adherent) and U937 (suspension) cell lines with different culture conditions, and measured their levels of the seven targets, while performed the corresponding ELISA (FIG. 7F). Results displayed by the 7-plex BLISA were well correlated with the ELISA results (r=0.9241, P=0.0010; r=0.9541, P=0.0002; r=9685, P<0.0001; r=7684, P=0.0259; r=0.9845, P<0.0001; r=0.9829, P<0.0001; r=0.7636, P=0.0275), showing the feasibility of multiplexed detection in cell lysates by MB-based BLISA.

1. A composition comprising an antibody linked with an oligonucleotide via a disulfide bond, wherein the oligonucleotide is specific for the antibody.

2. The composition according to claim 1, wherein the disulfide bond is capable of being cut by a reducing agent, preferably by Tris(2-carboxyethyl)phosphine, dithiothreitol or beta-mercaptoethanol, so as to release the oligonucleotide.

3. The composition according to claim 1, wherein the oligonucleotide comprises a hybridization chain reaction (HCR) initiator.

4. The composition according to claim 1, wherein the oligonucleotide comprises a first barcode sequence which is specific for the antibody that it is linked therewith.

5. The composition according to claim 4, wherein the oligonucleotide further comprises a unique molecular identifier (UMI) sequence, preferably, the UMI sequence comprises 10 to 20 nucleotides in length, more preferably 12 to 18 nucleotides in length.

6. The composition according to claim 4, wherein the first barcode sequence comprises 5 to 15 nucleotides in length, preferably 5 to 10 nucleotides in length.

7. The composition according to claim 4, wherein the oligonucleotide further comprises a second barcode sequence, which is specific for an information associated with a sample to be detected.

8. The composition according to claim 1, wherein the antibody is a secondary antibody, which is selected from a group consisting of an anti-IgE antibody, an anti-IgM antibody, an anti-IgG antibody, an anti-IgA antibody, and an anti-IgD antibody,
preferably, the oligonucleotide-associated antibody is a nanobody.

9. The composition according to claim 8, wherein the oligonucleotide-associated antibody specifically binds to an Fc region of the anti-IgE, anti-IgM, anti-IgG, anti-IgA or anti-IgD antibody.

10. A kit for detecting a plurality of targets, comprising the composition of claim 1.

11. The kit according to claim 10, wherein the kit comprises a plurality of oligonucleotide-associated antibodies each comprising an antibody associated with a specific HCR initiator, and one or more amplifiers,
preferably, each amplifier is conjugated with a detectable label selected from a group consisting of a chemical group, such as biotin, digoxigenin, acrydite, amine, succinimidyl ester, thiol, azide, TCO, Tetrazine, Alkyne and DBCO; and/or a fluorophore such as FITC, Cyanine dyes, Dylight fluors, Atto dyes, Janelia Fluor dyes, Alexa Fluor dyes.

12. A method for detecting one or more target in a sample, comprising a step of a) incubating a first primary antibody with the composition as defined in claim 1, to form a complex having the first primary antibody associated with the composition.

13. The method according to claim 12, wherein the antibody of the composition is a secondary antibody, and the complex is formed by the interaction between the first primary antibody and the secondary antibody.

14. The method according to claim 12, further comprising the steps of:
b) contacting the sample with the complex;
c) performing an amplification reaction to produce detectable amplified products; and
d) optionally, adding a reducing agent and incubating to cut the disulfide bond, and repeating the steps a) to c).

15. The method according to claim 14, wherein the oligonucleotide comprises an HCR initiator, and the amplification reaction in step c) is a hybridization chain reaction.

16. The method according to claim 12, further comprising the steps of:
b) contacting the sample with the complex;
c) adding a reducing agent and incubating to cut the disulfide bond;
d) collecting the specific oligonucleotides and performing an amplification reaction to detect the one or more targets; and
e) optionally, repeating steps a) to d),
wherein the oligonucleotide comprises a first barcode sequence.

17. The method according to claim 12, wherein the method comprises, before step b), a step of incubating the sample with a support which is associated with a target-specific binding substance, to capture the target in the sample, preferably the target-specific binding substance is a second primary antibody or an antigen.

18. The method according to claim 17, wherein the support is selected from a group consisting of a microplate and magnetic beads.

19. The method according to claim 12, wherein, in step a), one or more first primary antibodies incubate with one or more compositions each linked with an oligonucleotide specific for the antibody that it is linked therewith.

20. The method according to claim 12, wherein the method is used for high-throughput sequencing.


SEQUENCE LISTING (274 sequences)

SEQ ID NO: 1 - DNA, 41 bp, synthetic construct
  source 1..41: mol_type = other DNA
atatagcattctttcttgaggagggcagcaaacgggaagag

SEQ ID NO: 2 - DNA, 41 bp, synthetic construct
  source 1..41: mol_type = other DNA
atataagctcagtccatcctcgtaaatcctcatcaatcatc

SEQ ID NO: 3 - DNA, 41 bp, synthetic construct
  source 1..41: mol_type = other DNA
atataaaagtctaatccgtccctgcctctatatctccactc

SEQ ID NO: 4 - DNA, 41 bp, synthetic construct
  source 1..41: mol_type = other DNA
atatacacatttacagacctcaacctacctccaactctcac

SEQ ID NO: 5 - DNA, 41 bp, synthetic construct
  source 1..41: mol_type = other DNA
atatacacttcatatcactcactcccaatctctatctaccc

SEQ ID NO: 6 - DNA, 41 bp, synthetic construct
  source 1..41: mol_type = other DNA
atataccttgattatctcgtctccatcactcgcactctacc

SEQ ID NO: 7 - DNA, 41 bp, synthetic construct
  source 1..41: mol_type = other DNA
atatacacgtatctactccactctcagcacactcccaaccc

SEQ ID NO: 8 - DNA, 41 bp, synthetic construct
  source 1..41: mol_type = other DNA
atatacctcaagatactcctctacctactcgactaccctag

SEQ ID NO: 9 - DNA, 41 bp, synthetic construct
  source 1..41: mol_type = other DNA
atatacgcttagatatcactcctacgtcgaccacactcatc

SEQ ID NO: 10 - DNA, 41 bp, synthetic construct
  source 1..41: mol_type = other DNA
atataaggtaacgccttcctgctttatgctcaacatacaac

SEQ ID NO: 11 - DNA, 41 bp, synthetic construct
  source 1..41: mol_type = other DNA
atataaatgtcaatagcgagcgaccctatatttctgcacag

SEQ ID NO: 12 - DNA, 41 bp, synthetic construct
  source 1..41: mol_type = other DNA
atatacagattaacacaccacaaggtatctcgaacactctc

SEQ ID NO: 13 - DNA, 41 bp, synthetic construct
  source 1..41: mol_type = other DNA
atatacgattgtttgttgtggacgcatgctaatcggatgag

SEQ ID NO: 14 - DNA, 72 bp, synthetic construct
  source 1..72: mol_type = other DNA
cgtaaaggaagactcttcccgtttgctgccctcctcgcattctttcttgaggagggcagcaaacgggaagag

SEQ ID NO: 15 - DNA, 72 bp, synthetic construct
  source 1..72: mol_type = other DNA
gaggagggcagcaaacgggaagagtcttcctttacgctcttcccgtttgctgccctcctcaagaaagaatgc

SEQ ID NO: 16 - DNA, 72 bp, synthetic construct
  source 1..72: mol_type = other DNA
ggcggtttactggatgattgatgaggatttacgaggagctcagtccatcctcgtaaatcctcatcaatcatc

SEQ ID NO: 17 - DNA, 72 bp, synthetic construct
  source 1..72: mol_type = other DNA
cctcgtaaatcctcatcaatcatccagtaaaccgccgatgattgatgaggatttacgaggatggactgagct

SEQ ID NO: 18 - DNA, 72 bp, synthetic construct
  source 1..72: mol_type = other DNA
cgggttaaagttgagtggagatatagaggcagggacaaagtctaatccgtccctgcctctatatctccactc

SEQ ID NO: 19 - DNA, 72 bp, synthetic construct
  source 1..72: mol_type = other DNA
gtccctgcctctatatctccactcaactttaacccggagtggagatatagaggcagggacggattagacttt

SEQ ID NO: 20 - DNA, 72 bp, synthetic construct
  source 1..72: mol_type = other DNA
gaagcgaatatggtgagagttggaggtaggttgaggcacatttacagacctcaacctacctccaactctcac

SEQ ID NO: 21 - DNA, 72 bp, synthetic construct
  source 1..72: mol_type = other DNA
cctcaacctacctccaactctcaccatattcgcttcgtgagagttggaggtaggttgaggtctgtaaatgtg

SEQ ID NO: 22 - DNA, 72 bp, synthetic construct
  source 1..72: mol_type = other DNA
attggatttgtagggtagatagagattgggagtgagcacttcatatcactcactcccaatctctatctaccc

SEQ ID NO: 23 - DNA, 72 bp, synthetic construct
  source 1..72: mol_type = other DNA
ctcactcccaatctctatctaccctacaaatccaatgggtagatagagattgggagtgagtgatatgaagtg

SEQ ID NO: 24 - DNA, 72 bp, synthetic construct
  source 1..72: mol_type = other DNA
cgtctccatcactcgcactctaccgtcaagtcaaacggtagagtgcgagtgatggagacgagataatcaagg

SEQ ID NO: 25 - DNA, 72 bp, synthetic construct
  source 1..72: mol_type = other DNA
gtttgacttgacggtagagtgcgagtgatggagacgccttgattatctcgtctccatcactcgcactctacc

SEQ ID NO: 26 - DNA, 72 bp, synthetic construct
  source 1..72: mol_type = other DNA
ccactctcagcacactcccaaccctactacaagctcgggttgggagtgtgctgagagtggagtagatacgtg

SEQ ID NO: 27 - DNA, 72 bp, synthetic construct
  source 1..72: mol_type = other DNA
gagcttgtagtagggttgggagtgtgctgagagtggcacgtatctactccactctcagcacactcccaaccc

SEQ ID NO: 28 - DNA, 72 bp, synthetic construct
  source 1..72: mol_type = other DNA
cctctacctactcgactaccctagccgtaacttcacctagggtagtcgagtaggtagaggagtatcttgagg

SEQ ID NO: 29 - DNA, 72 bp, synthetic construct
  source 1..72: mol_type = other DNA
gtgaagttacggctagggtagtcgagtaggtagaggcctcaagatactcctctacctactcgactaccctag

SEQ ID NO: 30 - DNA, 72 bp, synthetic construct
  source 1..72: mol_type = other DNA
actcctacgtcgaccacactcatcctgcatgttcccgatgagtgtggtcgacgtaggagtgatatctaagcg

SEQ ID NO: 31 - DNA, 72 bp, synthetic construct
  source 1..72: mol_type = other DNA
gggaacatgcaggatgagtgtggtcgacgtaggagtcgcttagatatcactcctacgtcgaccacactcatc

SEQ ID NO: 32 - DNA, 72 bp, synthetic construct
  source 1..72: mol_type = other DNA
cctgctttatgctcaacatacaaccagaaatgcggcgttgtatgttgagcataaagcaggaaggcgttacct

SEQ ID NO: 33 - DNA, 72 bp, synthetic construct
  source 1..72: mol_type = other DNA
gccgcatttctggttgtatgttgagcataaagcaggaggtaacgccttcctgctttatgctcaacatacaac

SEQ ID NO: 34 - DNA, 72 bp, synthetic construct
  source 1..72: mol_type = other DNA
gagcgaccctatatttctgcacagaagttataccggctgtgcagaaatatagggtcgctcgctattgacatt

SEQ ID NO: 35 - DNA, 72 bp, synthetic construct
  source 1..72: mol_type = other DNA
ccggtataacttctgtgcagaaatatagggtcgctcaatgtcaatagcgagcgaccctatatttctgcacag

SEQ ID NO: 36 - DNA, 72 bp, synthetic construct
  source 1..72: mol_type = other DNA
ccacaaggtatctcgaacactctccaaattggctacgagagtgttcgagataccttgtggtgtgttaatctg

SEQ ID NO: 37 - DNA, 72 bp, synthetic construct
  source 1..72: mol_type = other DNA
gtagccaatttggagagtgttcgagataccttgtggcagattaacacaccacaaggtatctcgaacactctc

SEQ ID NO: 38 - DNA, 72 bp, synthetic construct
  source 1..72: mol_type = other DNA
gtggacacctgctaatcggatgagtgttcgttatcgctcatccgattagcaggtgtccacaacaaacaatcg

SEQ ID NO: 39 - DNA, 72 bp, synthetic construct
  source 1..72: mol_type = other DNA
cgataacgaacactcatccgattagcaggtgtccaccgattgtttgttgtggacacctgctaatcggatgag

SEQ ID NO: 40 - DNA, 62 bp, synthetic construct
  source 1..62: mol_type = other DNA
tcttgtggaaaggacgaaacacgtgatnnnnnnnnnnnnnnngtctggagcatgcgctttag

SEQ ID NO: 41 - DNA, 62 bp, synthetic construct
  source 1..62: mol_type = other DNA
tacacgacgctcttccgatctcgtgatnnnnnnnnnnnnnnnttgaaaaagtggcaccgagt

SEQ ID NO: 42 - DNA, 62 bp, synthetic construct
  source 1..62: mol_type = other DNA
acacgtctgaactccagtcaccgtgatnnnnnnnnnnnnnnncgtatgccgtcttctgcttg

SEQ ID NO: 43 - DNA, 62 bp, synthetic construct
  source 1..62: mol_type = other DNA
gacagttcgagtttgaagcgcgtgatnnnnnnnnnnnnnnnctagacgtgggagtgcatact

SEQ ID NO: 44 - DNA, 62 bp, synthetic construct
  source 1..62: mol_type = other DNA
gaaagatctggctgccatgccgtgatnnnnnnnnnnnnnnntcgcaaacctggttggaatca

SEQ ID NO: 45 - DNA, 62 bp, synthetic construct
  source 1..62: mol_type = other DNA
agatgacgtcgattgttggtcgtgatnnnnnnnnnnnnnnncatggaggttgtgtcaccgta

SEQ ID NO: 46 - DNA, 62 bp, synthetic construct
  source 1..62: mol_type = other DNA
tcaggtgcataggagtcagccgtgatnnnnnnnnnnnnnnnatgctgtcagttcatggctcc

SEQ ID NO: 47 - DNA, 62 bp, synthetic construct
  source 1..62: mol_type = other DNA
aacgttagtggacagaggctcgtgatnnnnnnnnnnnnnnnggtagagttcaaggacgcaca

SEQ ID NO: 48 - DNA, 62 bp, synthetic construct; identical to SEQ ID NO: 40
  source 1..62: mol_type = other DNA

SEQ ID NO: 49 - DNA, 62 bp, synthetic construct
  source 1..62: mol_type = other DNA
tcttgtggaaaggacgaaacaatcacgnnnnnnnnnnnnnnngtctggagcatgcgctttag

SEQ ID NO: 50 - DNA, 62 bp, synthetic construct
  source 1..62: mol_type = other DNA
tcttgtggaaaggacgaaacacgatgtnnnnnnnnnnnnnnngtctggagcatgcgctttag

SEQ ID NO: 51 - DNA, 62 bp, synthetic construct
  source 1..62: mol_type = other DNA
tcttgtggaaaggacgaaacattaggcnnnnnnnnnnnnnnngtctggagcatgcgctttag

SEQ ID NO: 52 - DNA, 62 bp, synthetic construct
  source 1..62: mol_type = other DNA
tcttgtggaaaggacgaaacatgaccannnnnnnnnnnnnnngtctggagcatgcgctttag

SEQ ID NO: 53 - DNA, 62 bp, synthetic construct
  source 1..62: mol_type = other DNA
tcttgtggaaaggacgaaacaacagtgnnnnnnnnnnnnnnngtctggagcatgcgctttag

SEQ ID NO: 54 - DNA, 62 bp, synthetic construct
  source 1..62: mol_type = other DNA
tcttgtggaaaggacgaaacagccaatnnnnnnnnnnnnnnngtctggagcatgcgctttag

SEQ ID NO: 55 - DNA, 62 bp, synthetic construct
  source 1..62: mol_type = other DNA
tcttgtggaaaggacgaaacacagatcnnnnnnnnnnnnnnngtctggagcatgcgctttag

SEQ ID NO: 56 - DNA, 62 bp, synthetic construct; identical to SEQ ID NO: 53
  source 1..62: mol_type = other DNA

SEQ ID NO: 57 - DNA, 62 bp, synthetic construct; identical to SEQ ID NO: 54
  source 1..62: mol_type = other DNA

SEQ ID NO: 58 - DNA, 62 bp, synthetic construct; identical to SEQ ID NO: 55
  source 1..62: mol_type = other DNA

SEQ ID NO: 59 - DNA, 62 bp, synthetic construct
  source 1..62: mol_type = other DNA
tcttgtggaaaggacgaaacaacttgannnnnnnnnnnnnnngtctggagcatgcgctttag

SEQ ID NO: 60 - DNA, 62 bp, synthetic construct
  source 1..62: mol_type = other DNA
tcttgtggaaaggacgaaacagatcagnnnnnnnnnnnnnnngtctggagcatgcgctttag

SEQ ID NO: 61 - protein, 126 aa, synthetic construct
MQVQLVECGGGLVQAGDSLRLSCVASGRSLDGATMRWYRQAPGKEREFVAGIFWDEIGTEYADTAKGRFTISRDNAKNTIYLQMTNLRSEDTAMYYCNGLVFGGEYWGKGTLVTVSSNGLHHHHHH

SEQ ID NO: 62 - protein, 130 aa, synthetic construct
MQVQLVECGGGLVQPGGSLRLSCAASGFTFSDTWMNWVRQAPGKGLYWISAINPDGGNTAYADSVKGRFTISRDNAKNMVYLQMDNLRPEDTAMYYCAKGWVRLPDPDLVRGQGTQVTVSSNGLHHHHHH

SEQ ID NO: 63 - DNA, 46 bp, synthetic construct
  source 1..46: mol_type = other DNA
aatgatacggcgaccaccgagatctacactctttccctacacgacg

SEQ ID NO: 64 - DNA, 48 bp, synthetic construct
  source 1..48: mol_type = other DNA
caagcagaagacggcatacgagatgcctaagtgactggagttcagacg

SEQ ID NO: 65 - DNA, 56 bp, synthetic construct
  source 1..56: mol_type = other DNA
ctctttccctacacgacgctcttccgatctatcacgcttgtggaaaggacgaaaca

SEQ ID NO: 66 - DNA, 56 bp, synthetic construct
  source 1..56: mol_type = other DNA
ctctttccctacacgacgctcttccgatctcgatgtcttgtggaaaggacgaaaca

SEQ ID NO: 67 - DNA, 56 bp, synthetic construct
  source 1..56: mol_type = other DNA
ctctttccctacacgacgctcttccgatctttaggccttgtggaaaggacgaaaca

SEQ ID NO: 68 - DNA, 56 bp, synthetic construct
  source 1..56: mol_type = other DNA
ctctttccctacacgacgctcttccgatcttgaccacttgtggaaaggacgaaaca

SEQ ID NO: 69 - DNA, 56 bp, synthetic construct
  source 1..56: mol_type = other DNA
ctctttccctacacgacgctcttccgatctacagtgcttgtggaaaggacgaaaca

SEQ ID NO: 70 - DNA, 56 bp, synthetic construct
  source 1..56: mol_type = other DNA
ctctttccctacacgacgctcttccgatctgccaatcttgtggaaaggacgaaaca

SEQ ID NO: 71 - DNA, 56 bp, synthetic construct
  source 1..56: mol_type = other DNA
ctctttccctacacgacgctcttccgatctcagatccttgtggaaaggacgaaaca

SEQ ID NO: 72 - DNA, 56 bp, synthetic construct
  source 1..56: mol_type = other DNA
ctctttccctacacgacgctcttccgatctacttgacttgtggaaaggacgaaaca

SEQ ID NO: 73 - DNA, 56 bp, synthetic construct
  source 1..56: mol_type = other DNA
ctctttccctacacgacgctcttccgatctgatcagcttgtggaaaggacgaaaca

SEQ ID NO: 74 - DNA, 56 bp, synthetic construct
  source 1..56: mol_type = other DNA
ctctttccctacacgacgctcttccgatcttagcttcttgtggaaaggacgaaaca

SEQ ID NO: 75 - DNA, 56 bp, synthetic construct
  source 1..56: mol_type = other DNA
ctctttccctacacgacgctcttccgatctggctaccttgtggaaaggacgaaaca

SEQ ID NO: 76 - DNA, 56 bp, synthetic construct
  source 1..56: mol_type = other DNA
ctctttccctacacgacgctcttccgatctcttgtacttgtggaaaggacgaaaca

SEQ ID NO: 77 - DNA, 56 bp, synthetic construct
  source 1..56: mol_type = other DNA
ctctttccctacacgacgctcttccgatctagtcaacttgtggaaaggacgaaaca

SEQ ID NO: 78 - DNA, 56 bp, synthetic construct
  source 1..56: mol_type = other DNA
ctctttccctacacgacgctcttccgatctagttcccttgtggaaaggacgaaaca

SEQ ID NO: 79 - DNA, 56 bp, synthetic construct
  source 1..56: mol_type = other DNA
ctctttccctacacgacgctcttccgatctatgtcacttgtggaaaggacgaaaca

SEQ ID NO: 80 - DNA, 56 bp, synthetic construct
  source 1..56: mol_type = other DNA
ctctttccctacacgacgctcttccgatctccgtcccttgtggaaaggacgaaaca

SEQ ID NO: 81 - DNA, 56 bp, synthetic construct
  source 1..56: mol_type = other DNA
ctctttccctacacgacgctcttccgatctgtagagcttgtggaaaggacgaaaca

SEQ ID NO: 82 - DNA, 56 bp, synthetic construct
  source 1..56: mol_type = other DNA
ctctttccctacacgacgctcttccgatctgtccgccttgtggaaaggacgaaaca

SEQ ID NO: 83 - DNA, 56 bp, synthetic construct
  source 1..56: mol_type = other DNA
ctctttccctacacgacgctcttccgatctgtgaaacttgtggaaaggacgaaaca

SEQ ID NO: 84 - DNA, 56 bp, synthetic construct
  source 1..56: mol_type = other DNA
ctctttccctacacgacgctcttccgatctgtggcccttgtggaaaggacgaaaca

SEQ ID NO: 85 - DNA, 56 bp, synthetic construct
  source 1..56: mol_type = other DNA
ctctttccctacacgacgctcttccgatctgtttcgcttgtggaaaggacgaaaca

SEQ ID NO: 86 - DNA, 56 bp, synthetic construct
  source 1..56: mol_type = other DNA
ctctttccctacacgacgctcttccgatctcgtacgcttgtggaaaggacgaaaca

SEQ ID NO: 87 - DNA, 56 bp, synthetic construct
  source 1..56: mol_type = other DNA
ctctttccctacacgacgctcttccgatctgagtggcttgtggaaaggacgaaaca

SEQ ID NO: 88 - DNA, 56 bp, synthetic construct
  source 1..56: mol_type = other DNA
ctctttccctacacgacgctcttccgatctggtagccttgtggaaaggacgaaaca

SEQ ID NO: 89 - DNA, 56 bp, synthetic construct
  source 1..56: mol_type = other DNA
ctctttccctacacgacgctcttccgatctactgatcttgtggaaaggacgaaaca

SEQ ID NO: 90 - DNA, 56 bp, synthetic construct
  source 1..56: mol_type = other DNA
ctctttccctacacgacgctcttccgatctatgagccttgtggaaaggacgaaaca

SEQ ID NO: 91 - DNA, 56 bp, synthetic construct
  source 1..56: mol_type = other DNA
ctctttccctacacgacgctcttccgatctattcctcttgtggaaaggacgaaaca

SEQ ID NO: 92 - DNA, 56 bp, synthetic construct
  source 1..56: mol_type = other DNA
ctctttccctacacgacgctcttccgatctcaaaagcttgtggaaaggacgaaaca

SEQ ID NO: 93 - DNA, 56 bp, synthetic construct
  source 1..56: mol_type = other DNA
ctctttccctacacgacgctcttccgatctcaactacttgtggaaaggacgaaaca

SEQ ID NO: 94 - DNA, 56 bp, synthetic construct
  source 1..56: mol_type = other DNA
ctctttccctacacgacgctcttccgatctcaccggcttgtggaaaggacgaaaca

SEQ ID NO: 95 - DNA, 56 bp, synthetic construct
  source 1..56: mol_type = other DNA
ctctttccctacacgacgctcttccgatctcacgatcttgtggaaaggacgaaaca

SEQ ID NO: 96 - DNA, 56 bp, synthetic construct
  source 1..56: mol_type = other DNA
ctctttccctacacgacgctcttccgatctcactcacttgtggaaaggacgaaaca

SEQ ID NO: 97 - DNA, 56 bp, synthetic construct
  source 1..56: mol_type = other DNA
ctctttccctacacgacgctcttccgatctcaggcgcttgtggaaaggacgaaaca

SEQ ID NO: 98 - DNA, 56 bp, synthetic construct
  source 1..56: mol_type = other DNA
ctctttccctacacgacgctcttccgatctcatggccttgtggaaaggacgaaaca

SEQ ID NO: 99 - DNA, 56 bp, synthetic construct
  source 1..56: mol_type = other DNA
ctctttccctacacgacgctcttccgatctcattttcttgtggaaaggacgaaaca

SEQ ID NO: 100 - DNA, 56 bp, synthetic construct
  source 1..56: mol_type = other DNA
ctctttccctacacgacgctcttccgatctccaacacttgtggaaaggacgaaaca

SEQ ID NO: 101 - DNA, 56 bp, synthetic construct
  source 1..56: mol_type = other DNA
ctctttccctacacgacgctcttccgatctcggaatcttgtggaaaggacgaaaca

SEQ ID NO: 102 - DNA, 56 bp, synthetic construct
  source 1..56: mol_type = other DNA
ctctttccctacacgacgctcttccgatctctagctcttgtggaaaggacgaaaca

SEQ ID NO: 103 - DNA, 56 bp, synthetic construct
  source 1..56: mol_type = other DNA
ctctttccctacacgacgctcttccgatctctataccttgtggaaaggacgaaaca

SEQ ID NO: 104 - DNA, 56 bp, synthetic construct
  source 1..56: mol_type = other DNA
ctctttccctacacgacgctcttccgatctctcagacttgtggaaaggacgaaaca

SEQ ID NO: 105 - DNA, 56 bp, synthetic construct
  source 1..56: mol_type = other DNA
ctctttccctacacgacgctcttccgatctgacgaccttgtggaaaggacgaaaca

SEQ ID NO: 106 - DNA, 56 bp, synthetic construct
  source 1..56: mol_type = other DNA
ctctttccctacacgacgctcttccgatcttaatcgcttgtggaaaggacgaaaca

SEQ ID NO: 107 - DNA, 56 bp, synthetic construct
  source 1..56: mol_type = other DNA
ctctttccctacacgacgctcttccgatcttacagccttgtggaaaggacgaaaca

SEQ ID NO: 108 - DNA, 56 bp, synthetic construct
  source 1..56: mol_type = other DNA
ctctttccctacacgacgctcttccgatcttataatcttgtggaaaggacgaaaca

SEQ ID NO: 109 - DNA, 56 bp, synthetic construct
  source 1..56: mol_type = other DNA
ctctttccctacacgacgctcttccgatcttcattccttgtggaaaggacgaaaca

SEQ ID NO: 110 - DNA, 56 bp, synthetic construct
  source 1..56: mol_type = other DNA
ctctttccctacacgacgctcttccgatcttcccgacttgtggaaaggacgaaaca

SEQ ID NO: 111 - DNA, 56 bp, synthetic construct
  source 1..56: mol_type = other DNA
ctctttccctacacgacgctcttccgatcttcgaagcttgtggaaaggacgaaaca

SEQ ID NO: 112 - DNA, 56 bp, synthetic construct
  source 1..56: mol_type = other DNA
ctctttccctacacgacgctcttccgatcttcggcacttgtggaaaggacgaaaca

SEQ ID NO: 113 - DNA, 56 bp, synthetic construct
  source 1..56: mol_type = other DNA
ctctttccctacacgacgctcttccgatctgtacttcttgtggaaaggacgaaaca

SEQ ID NO: 114 - DNA, 56 bp, synthetic construct
  source 1..56: mol_type = other DNA
ctctttccctacacgacgctcttccgatctatggcacttgtggaaaggacgaaaca

SEQ ID NO: 115 - DNA, 56 bp, synthetic construct
  source 1..56: mol_type = other DNA
ctctttccctacacgacgctcttccgatctaaggagcttgtggaaaggacgaaaca

SEQ ID NO: 116 - DNA, 56 bp, synthetic construct
  source 1..56: mol_type = other DNA
ctctttccctacacgacgctcttccgatctctatctcttgtggaaaggacgaaaca

SEQ ID NO: 117 - DNA, 56 bp, synthetic construct
  source 1..56: mol_type = other DNA
ctctttccctacacgacgctcttccgatcttatatccttgtggaaaggacgaaaca

SEQ ID NO: 118 - DNA, 56 bp, synthetic construct
  source 1..56: mol_type = other DNA
ctctttccctacacgacgctcttccgatcttattatcttgtggaaaggacgaaaca

SEQ ID NO: 119 - DNA, 56 bp, synthetic construct
  source 1..56: mol_type = other DNA
ctctttccctacacgacgctcttccgatctagcacccttgtggaaaggacgaaaca

SEQ ID NO: 120 - DNA, 56 bp, synthetic construct
  source 1..56: mol_type = other DNA
ctctttccctacacgacgctcttccgatcttccacacttgtggaaaggacgaaaca

SEQ ID NO: 121 - DNA, 56 bp, synthetic construct
  source 1..56: mol_type = other DNA
ctctttccctacacgacgctcttccgatctttctcgcttgtggaaaggacgaaaca

SEQ ID NO: 122 - DNA, 56 bp, synthetic construct
  source 1..56: mol_type = other DNA
ctctttccctacacgacgctcttccgatcttcctgccttgtggaaaggacgaaaca

SEQ ID NO: 123 - DNA, 56 bp, synthetic construct
  source 1..56: mol_type = other DNA
ctctttccctacacgacgctcttccgatctcgcggccttgtggaaaggacgaaaca

SEQ ID NO: 124 - DNA, 56 bp, synthetic construct
  source 1..56: mol_type = other DNA
ctctttccctacacgacgctcttccgatcttgcgctcttgtggaaaggacgaaaca

SEQ ID NO: 125 - DNA, 56 bp, synthetic construct
  source 1..56: mol_type = other DNA
ctctttccctacacgacgctcttccgatctaacggacttgtggaaaggacgaaaca

SEQ ID NO: 126 - DNA, 56 bp, synthetic construct
  source 1..56: mol_type = other DNA
ctctttccctacacgacgctcttccgatctgacgatcttgtggaaaggacgaaaca

SEQ ID NO: 127 - DNA, 56 bp, synthetic construct
  source 1..56: mol_type = other DNA
ctctttccctacacgacgctcttccgatctttgcggcttgtggaaaggacgaaaca

SEQ ID NO: 128 - DNA, 56 bp, synthetic construct
  source 1..56: mol_type = other DNA
ctctttccctacacgacgctcttccgatctcaagttcttgtggaaaggacgaaaca

SEQ ID NO: 129 - DNA, 56 bp, synthetic construct
  source 1..56: mol_type = other DNA
ctctttccctacacgacgctcttccgatcttaccttcttgtggaaaggacgaaaca

SEQ ID NO: 130 - DNA, 56 bp, synthetic construct
  source 1..56: mol_type = other DNA
ctctttccctacacgacgctcttccgatctgagcgccttgtggaaaggacgaaaca

SEQ ID NO: 131 - DNA, 56 bp, synthetic construct
  source 1..56: mol_type = other DNA
ctctttccctacacgacgctcttccgatctacgaggcttgtggaaaggacgaaaca

SEQ ID NO: 132 - DNA, 56 bp, synthetic construct
  source 1..56: mol_type = other DNA
ctctttccctacacgacgctcttccgatctaatcgtcttgtggaaaggacgaaaca

SEQ ID NO: 133 - DNA, 56 bp, synthetic construct
  source 1..56: mol_type = other DNA
ctctttccctacacgacgctcttccgatctgagtctcttgtggaaaggacgaaaca

SEQ ID NO: 134 - DNA, 56 bp, synthetic construct
  source 1..56: mol_type = other DNA
ctctttccctacacgacgctcttccgatctagaccacttgtggaaaggacgaaaca

SEQ ID NO: 135 - DNA, 56 bp, synthetic construct
  source 1..56: mol_type = other DNA
ctctttccctacacgacgctcttccgatctaattcccttgtggaaaggacgaaaca

SEQ ID NO: 136 - DNA, 56 bp, synthetic construct
  source 1..56: mol_type = other DNA
ctctttccctacacgacgctcttccgatctagatgtcttgtggaaaggacgaaaca

SEQ ID NO: 137 - DNA, 56 bp, synthetic construct
  source 1..56: mol_type = other DNA
ctctttccctacacgacgctcttccgatctggagaacttgtggaaaggacgaaaca

SEQ ID NO: 138 - DNA, 56 bp, synthetic construct
  source 1..56: mol_type = other DNA
ctctttccctacacgacgctcttccgatctatagatcttgtggaaaggacgaaaca

SEQ ID NO: 139 - DNA, 56 bp, synthetic construct
  source 1..56: mol_type = other DNA
ctctttccctacacgacgctcttccgatcttatgcacttgtggaaaggacgaaaca

SEQ ID NO: 140 - DNA, 56 bp, synthetic construct
  source 1..56: mol_type = other DNA
ctctttccctacacgacgctcttccgatctcctattcttgtggaaaggacgaaaca

SEQ ID NO: 141 - DNA, 56 bp, synthetic construct
  source 1..56: mol_type = other DNA
ctctttccctacacgacgctcttccgatcttaaccgcttgtggaaaggacgaaaca

SEQ ID NO: 142 - DNA, 56 bp, synthetic construct
  source 1..56: mol_type = other DNA
ctctttccctacacgacgctcttccgatctagtctccttgtggaaaggacgaaaca

SEQ ID NO: 143 - DNA, 56 bp, synthetic construct
  source 1..56: mol_type = other DNA
ctctttccctacacgacgctcttccgatcttggagccttgtggaaaggacgaaaca

SEQ ID NO: 144 - DNA, 56 bp, synthetic construct
  source 1..56: mol_type = other DNA
ctctttccctacacgacgctcttccgatctctgcatcttgtggaaaggacgaaaca

SEQ ID NO: 145 - DNA, 56 bp, synthetic construct
  source 1..56: mol_type = other DNA
ctctttccctacacgacgctcttccgatctcaggcccttgtggaaaggacgaaaca

SEQ ID NO: 146 - DNA, 56 bp, synthetic construct
  source 1..56: mol_type = other DNA
ctctttccctacacgacgctcttccgatctcagtaacttgtggaaaggacgaaaca

SEQ ID NO: 147 - DNA, 56 bp, synthetic construct
  source 1..56: mol_type = other DNA
ctctttccctacacgacgctcttccgatctgttacccttgtggaaaggacgaaaca

SEQ ID NO: 148 - DNA, 56 bp, synthetic construct
  source 1..56: mol_type = other DNA
ctctttccctacacgacgctcttccgatctactgctcttgtggaaaggacgaaaca

SEQ ID NO: 149 - DNA, 56 bp, synthetic construct
  source 1..56: mol_type = other DNA
ctctttccctacacgacgctcttccgatctctaaggcttgtggaaaggacgaaaca

SEQ ID NO: 150 - DNA, 56 bp, synthetic construct
  source 1..56: mol_type = other DNA
ctctttccctacacgacgctcttccgatctatccaacttgtggaaaggacgaaaca

SEQ ID NO: 151 - DNA, 56 bp, synthetic construct
  source 1..56: mol_type = other DNA
ctctttccctacacgacgctcttccgatctgtgagacttgtggaaaggacgaaaca

SEQ ID NO: 152 - DNA, 56 bp, synthetic construct
  source 1..56: mol_type = other DNA
ctctttccctacacgacgctcttccgatcttaagaccttgtggaaaggacgaaaca

SEQ ID NO: 153 - DNA, 56 bp, synthetic construct
  source 1..56: mol_type = other DNA
ctctttccctacacgacgctcttccgatctcgttagcttgtggaaaggacgaaaca

SEQ ID NO: 154 - DNA, 56 bp, synthetic construct
  source 1..56: mol_type = other DNA
ctctttccctacacgacgctcttccgatctgtgtatcttgtggaaaggacgaaaca

SEQ ID NO: 155 - DNA, 56 bp, synthetic construct
  source 1..56: mol_type = other DNA
ctctttccctacacgacgctcttccgatctgaatgccttgtggaaaggacgaaaca

SEQ ID NO: 156 - DNA, 56 bp, synthetic construct
  source 1..56: mol_type = other DNA
ctctttccctacacgacgctcttccgatctagagtacttgtggaaaggacgaaaca

SEQ ID NO: 157 - DNA, 56 bp, synthetic construct
  source 1..56: mol_type = other DNA
ctctttccctacacgacgctcttccgatctagtccacttgtggaaaggacgaaaca

SEQ ID NO: 158 - DNA, 56 bp, synthetic construct
  source 1..56: mol_type = other DNA
ctctttccctacacgacgctcttccgatctctatggcttgtggaaaggacgaaaca

SEQ ID NO: 159 - DNA, 56 bp, synthetic construct
  source 1..56: mol_type = other DNA
ctctttccctacacgacgctcttccgatctaccttacttgtggaaaggacgaaaca

SEQ ID NO: 160 - DNA, 56 bp, synthetic construct
  source 1..56: mol_type = other DNA
ctctttccctacacgacgctcttccgatctcgccaacttgtggaaaggacgaaaca

SEQ ID NO: 161 - DNA, 54 bp, synthetic construct
  source 1..54: mol_type = other DNA
gtgactggagttcagacgtgtgctcttccgatctctaaagcgcatgctccagac

SEQ ID NO: 162 - DNA, 49 bp, synthetic construct
  source 1..49: mol_type = other DNA
gacaagtggccacaaaccaccagatcacgcttgtggaaaggacgaaaca

SEQ ID NO: 163 - DNA, 49 bp, synthetic construct
  source 1..49: mol_type = other DNA
gacaagtggccacaaaccaccagcgatgtcttgtggaaaggacgaaaca

SEQ ID NO: 164 - DNA, 49 bp, synthetic construct
  source 1..49: mol_type = other DNA
gacaagtggccacaaaccaccagttaggccttgtggaaaggacgaaaca

SEQ ID NO: 165 - DNA, 49 bp, synthetic construct
  source 1..49: mol_type = other DNA
gacaagtggccacaaaccaccagtgaccacttgtggaaaggacgaaaca

SEQ ID NO: 166 - DNA, 49 bp, synthetic construct
  source 1..49: mol_type = other DNA
gacaagtggccacaaaccaccagacagtgcttgtggaaaggacgaaaca

SEQ ID NO: 167 - DNA, 49 bp, synthetic construct
  source 1..49: mol_type = other DNA
gacaagtggccacaaaccaccaggccaatcttgtggaaaggacgaaaca

SEQ ID NO: 168 - DNA, 49 bp, synthetic construct
  source 1..49: mol_type = other DNA
gacaagtggccacaaaccaccagcagatccttgtggaaaggacgaaaca

SEQ ID NO: 169 - DNA, 49 bp, synthetic construct
  source 1..49: mol_type = other DNA
gacaagtggccacaaaccaccagacttgacttgtggaaaggacgaaaca

SEQ ID NO: 170 - DNA, 49 bp, synthetic construct
  source 1..49: mol_type = other DNA
gacaagtggccacaaaccaccaggatcagcttgtggaaaggacgaaaca

SEQ ID NO: 171 - DNA, 49 bp, synthetic construct
  source 1..49: mol_type = other DNA
gacaagtggccacaaaccaccagtagcttcttgtggaaaggacgaaaca

SEQ ID NO: 172 - DNA, 49 bp, synthetic construct
  source 1..49: mol_type = other DNA
gacaagtggccacaaaccaccagggctaccttgtggaaaggacgaaaca

SEQ ID NO: 173 - DNA, 49 bp, synthetic construct
  source 1..49: mol_type = other DNA
gacaagtggccacaaaccaccagcttgtacttgtggaaaggacgaaaca

SEQ ID NO: 174 - DNA, 49 bp, synthetic construct
  source 1..49: mol_type = other DNA
gacaagtggccacaaaccaccagagtcaacttgtggaaaggacgaaaca

SEQ ID NO: 175 - DNA, 49 bp, synthetic construct
  source 1..49: mol_type = other DNA
gacaagtggccacaaaccaccagagttcccttgtggaaaggacgaaaca

SEQ ID NO: 176 - DNA, 49 bp, synthetic construct
  source 1..49: mol_type = other DNA
gacaagtggccacaaaccaccagatgtcacttgtggaaaggacgaaaca

SEQ ID NO: 177 - DNA, 49 bp, synthetic construct
  source 1..49: mol_type = other DNA
gacaagtggccacaaaccaccagccgtcccttgtggaaaggacgaaaca

SEQ ID NO: 178 - DNA, 49 bp, synthetic construct
  source 1..49: mol_type = other DNA
gacaagtggccacaaaccaccaggtagagcttgtggaaaggacgaaaca

SEQ ID NO: 179 - DNA, 49 bp, synthetic construct
  source 1..49: mol_type = other DNA
gacaagtggccacaaaccaccaggtccgccttgtggaaaggacgaaaca

SEQ ID NO: 180 - DNA, 49 bp, synthetic construct
  source 1..49: mol_type = other DNA
gacaagtggccacaaaccaccaggtgaaacttgtggaaaggacgaaaca

SEQ ID NO: 181 - DNA, 49 bp, synthetic construct
  source 1..49: mol_type = other DNA
gacaagtggccacaaaccaccaggtggcccttgtggaaaggacgaaaca

SEQ ID NO: 182 - DNA, 49 bp, synthetic construct
  source 1..49: mol_type = other DNA
gacaagtggccacaaaccaccaggtttcgcttgtggaaaggacgaaaca

SEQ ID NO: 183 - DNA, 49 bp, synthetic construct
  source 1..49: mol_type = other DNA
gacaagtggccacaaaccaccagcgtacgcttgtggaaaggacgaaaca

SEQ ID NO: 184 - DNA, 49 bp, synthetic construct
  source 1..49: mol_type = other DNA
gacaagtggccacaaaccaccaggagtggcttgtggaaaggacgaaaca

SEQ ID NO: 185 - DNA, 49 bp, synthetic construct
  source 1..49: mol_type = other DNA
gacaagtggccacaaaccaccagggtagccttgtggaaaggacgaaaca

SEQ ID NO: 186 - DNA, 49 bp, synthetic construct
  source 1..49: mol_type = other DNA
gacaagtggccacaaaccaccagactgatcttgtggaaaggacgaaaca

SEQ ID NO: 187 - DNA, 49 bp, synthetic construct
  source 1..49: mol_type = other DNA
gacaagtggccacaaaccaccagatgagccttgtggaaaggacgaaaca

SEQ ID NO: 188 - DNA, 49 bp, synthetic construct
  source 1..49: mol_type = other DNA
gacaagtggccacaaaccaccagattcctcttgtggaaaggacgaaaca

SEQ ID NO: 189 - DNA, 49 bp, synthetic construct
  source 1..49: mol_type = other DNA
gacaagtggccacaaaccaccagcaaaagcttgtggaaaggacgaaaca

SEQ ID NO: 190 - DNA, 49 bp, synthetic construct
  source 1..49: mol_type = other DNA
gacaagtggccacaaaccaccagcaactacttgtggaaaggacgaaaca

SEQ ID NO: 191 - DNA, 49 bp, synthetic construct
  source 1..49: mol_type = other DNA
gacaagtggccacaaaccaccagcaccggcttgtggaaaggacgaaaca

SEQ ID NO: 192 - DNA, 49 bp, synthetic construct
  source 1..49: mol_type = other DNA
gacaagtggccacaaaccaccagcacgatcttgtggaaaggacgaaaca

SEQ ID NO: 193 - DNA, 49 bp, synthetic construct
  source 1..49: mol_type = other DNA
gacaagtggccacaaaccaccagcactcacttgtggaaaggacgaaaca

SEQ ID NO: 194 - DNA, 49 bp, synthetic construct
  source 1..49: mol_type = other DNA
gacaagtggccacaaaccaccagcaggcgcttgtggaaaggacgaaaca

SEQ ID NO: 195 - DNA, 49 bp, synthetic construct
  source 1..49: mol_type = other DNA
gacaagtggccacaaaccaccagcatggccttgtggaaaggacgaaaca

SEQ ID NO: 196 - DNA, 49 bp, synthetic construct
  source 1..49: mol_type = other DNA
gacaagtggccacaaaccaccagcattttcttgtggaaaggacgaaaca

SEQ ID NO: 197 - DNA, 49 bp, synthetic construct
  source 1..49: mol_type = other DNA
gacaagtggccacaaaccaccagccaacacttgtggaaaggacgaaaca

SEQ ID NO: 198 - DNA, 49 bp, synthetic construct
  source 1..49: mol_type = other DNA
gacaagtggccacaaaccaccagcggaatcttgtggaaaggacgaaaca

SEQ ID NO: 199 - DNA, 49 bp, synthetic construct
  source 1..49: mol_type = other DNA
gacaagtggccacaaaccaccagctagctcttgtggaaaggacgaaaca

SEQ ID NO: 200 - DNA, 49 bp, synthetic construct
  source 1..49: mol_type = other DNA
gacaagtggccacaaaccaccagctataccttgtggaaaggacgaaaca

SEQ ID NO: 201 - DNA, 49 bp, synthetic construct
  source 1..49: mol_type = other DNA
gacaagtggccacaaaccaccagctcagacttgtggaaaggacgaaaca

SEQ ID NO: 202 - DNA, 49 bp, synthetic construct
  source 1..49: mol_type = other DNA
gacaagtggccacaaaccaccaggacgaccttgtggaaaggacgaaaca

SEQ ID NO: 203 - DNA, 49 bp, synthetic construct
  source 1..49: mol_type = other DNA
gacaagtggccacaaaccaccagtaatcgcttgtggaaaggacgaaaca

SEQ ID NO: 204 - DNA, 49 bp, synthetic construct
  source 1..49: mol_type = other DNA
gacaagtggccacaaaccaccagtacagccttgtggaaaggacgaaaca

SEQ ID NO: 205 - DNA, 49 bp, synthetic construct
  source 1..49: mol_type = other DNA
gacaagtggccacaaaccaccagtataatcttgtggaaaggacgaaaca

SEQ ID NO: 206 - DNA, 49 bp, synthetic construct
  source 1..49: mol_type = other DNA
gacaagtggccacaaaccaccagtcattccttgtggaaaggacgaaaca

SEQ ID NO: 207 - DNA, 49 bp, synthetic construct
  source 1..49: mol_type = other DNA
gacaagtggccacaaaccaccagtcccgacttgtggaaaggacgaaaca

SEQ ID NO: 208 - DNA, 49 bp, synthetic construct
  source 1..49: mol_type = other DNA
gacaagtggccacaaaccaccagtcgaagcttgtggaaaggacgaaaca

SEQ ID NO: 209 - DNA, 49 bp, synthetic construct
  source 1..49: mol_type = other DNA
gacaagtggccacaaaccaccagtcggcacttgtggaaaggacgaaaca

SEQ ID NO: 210 - DNA, 49 bp, synthetic construct
  source 1..49: mol_type = other DNA
gacaagtggccacaaaccaccaggtacttcttgtggaaaggacgaaaca

SEQ ID NO: 211 - DNA, 49 bp, synthetic construct
  source 1..49: mol_type = other DNA
gacaagtggccacaaaccaccagatggcacttgtggaaaggacgaaaca

SEQ ID NO: 212 - DNA, 49 bp, synthetic construct
  source 1..49: mol_type = other DNA
gacaagtggccacaaaccaccagaaggagcttgtggaaaggacgaaaca

SEQ ID NO: 213 - DNA, 49 bp, synthetic construct
  source 1..49: mol_type = other DNA
gacaagtggccacaaaccaccagctatctcttgtggaaaggacgaaaca

SEQ ID NO: 214 - DNA, 49 bp, synthetic construct
  source 1..49: mol_type = other DNA
gacaagtggccacaaaccaccagtatatccttgtggaaaggacgaaaca

SEQ ID NO: 215 - DNA, 49 bp, synthetic construct
  source 1..49: mol_type = other DNA
gacaagtggccacaaaccaccagtattatcttgtggaaaggacgaaaca

SEQ ID NO: 216 - DNA, 49 bp, synthetic construct
  source 1..49: mol_type = other DNA
gacaagtggccacaaaccaccagagcacccttgtggaaaggacgaaaca

SEQ ID NO: 217 - DNA, 49 bp, synthetic construct
  source 1..49: mol_type = other DNA
gacaagtggccacaaaccaccagtccacacttgtggaaaggacgaaaca

SEQ ID NO: 218 - DNA, 49 bp, synthetic construct
  source 1..49: mol_type = other DNA
gacaagtggccacaaaccaccagttctcgcttgtggaaaggacgaaaca

SEQ ID NO: 219 - DNA, 49 bp, synthetic construct
  source 1..49: mol_type = other DNA
gacaagtggccacaaaccaccagtcctgccttgtggaaaggacgaaaca

SEQ ID NO: 220 - DNA, 49 bp, synthetic construct
  source 1..49: mol_type = other DNA
gacaagtggccacaaaccaccagcgcggccttgtggaaaggacgaaaca

SEQ ID NO: 221 - DNA, 49 bp, synthetic construct
  source 1..49: mol_type = other DNA
gacaagtggccacaaaccaccagtgcgctcttgtggaaaggacgaaaca

SEQ ID NO: 222 - DNA, 49 bp, synthetic construct
  source 1..49: mol_type = other DNA
gacaagtggccacaaaccaccagaacggacttgtggaaaggacgaaaca

SEQ ID NO: 223 - DNA, 49 bp, synthetic construct
  source 1..49: mol_type = other DNA
gacaagtggccacaaaccaccaggacgatcttgtggaaaggacgaaaca

SEQ ID NO: 224 - DNA, 49 bp, synthetic construct
  source 1..49: mol_type = other DNA
gacaagtggccacaaaccaccagttgcggcttgtggaaaggacgaaaca

SEQ ID NO: 225 - DNA, 49 bp, synthetic construct
  source 1..49: mol_type = other DNA
gacaagtggccacaaaccaccagcaagttcttgtggaaaggacgaaaca

SEQ ID NO: 226 - DNA, 49 bp, synthetic construct
  source 1..49: mol_type = other DNA
gacaagtggccacaaaccaccagtaccttcttgtggaaaggacgaaaca

SEQ ID NO: 227 - DNA, 49 bp, synthetic construct
  source 1..49: mol_type = other DNA
gacaagtggccacaaaccaccaggagcgccttgtggaaaggacgaaaca

SEQ ID NO: 228 - DNA, 49 bp, synthetic construct
  source 1..49: mol_type = other DNA
gacaagtggccacaaaccaccagacgaggcttgtggaaaggacgaaaca

SEQ ID NO: 229 - DNA, 49 bp, synthetic construct
  source 1..49: mol_type = other DNA
gacaagtggccacaaaccaccagaatcgtcttgtggaaaggacgaaaca

SEQ ID NO: 230 - DNA, 49 bp, synthetic construct
  source 1..49: mol_type = other DNA
gacaagtggccacaaaccaccaggagtctcttgtggaaaggacgaaaca

SEQ ID NO: 231 - DNA, 49 bp, synthetic construct
  source 1..49: mol_type = other DNA
gacaagtggccacaaaccaccagagaccacttgtggaaaggacgaaaca

SEQ ID NO: 232 - DNA, 49 bp, synthetic construct
  source 1..49: mol_type = other DNA
gacaagtggccacaaaccaccagaattcccttgtggaaaggacgaaaca

SEQ ID NO: 233 - DNA, 49 bp, synthetic construct
  source 1..49: mol_type = other DNA
gacaagtggccacaaaccaccagagatgtcttgtggaaaggacgaaaca

SEQ ID NO: 234 - DNA, 49 bp, synthetic construct
  source 1..49: mol_type = other DNA
gacaagtggccacaaaccaccagggagaacttgtggaaaggacgaaaca

SEQ ID NO: 235 - DNA, 49 bp, synthetic construct
  source 1..49: mol_type = other DNA
gacaagtggccacaaaccaccagatagatcttgtggaaaggacgaaaca

SEQ ID NO: 236 - DNA, 49 bp, synthetic construct
  source 1..49: mol_type = other DNA
gacaagtggccacaaaccaccagtatgcacttgtggaaaggacgaaaca

SEQ ID NO: 237 - DNA, 49 bp, synthetic construct
  source 1..49: mol_type = other DNA
gacaagtggccacaaaccaccagcctattcttgtggaaaggacgaaaca

SEQ ID NO: 238 - DNA, 49 bp, synthetic construct
  source 1..49: mol_type = other DNA
gacaagtggccacaaaccaccagtaaccgcttgtggaaaggacgaaaca

SEQ ID NO: 239 - DNA, 49 bp, synthetic construct
  source 1..49: mol_type = other DNA
gacaagtggccacaaaccaccagagtctccttgtggaaaggacgaaaca

SEQ ID NO: 240 - DNA, 49 bp, synthetic construct
  source 1..49: mol_type = other DNA
gacaagtggccacaaaccaccagtggagccttgtggaaaggacgaaaca

SEQ ID NO: 241 - DNA, 49 bp, synthetic construct
  source 1..49: mol_type = other DNA
gacaagtggccacaaaccaccagctgcatcttgtggaaaggacgaaaca

SEQ ID NO: 242 - DNA, 49 bp, synthetic construct
  source 1..49: mol_type = other DNA
gacaagtggccacaaaccaccagcaggcccttgtggaaaggacgaaaca

SEQ ID NO: 243 - DNA, 49 bp, synthetic construct
  source 1..49: mol_type = other DNA
gacaagtggccacaaaccaccagcagtaacttgtggaaaggacgaaaca

SEQ ID NO: 244 - DNA, 49 bp, synthetic construct
  source 1..49: mol_type = other DNA
gacaagtggccacaaaccaccaggttacccttgtggaaaggacgaaaca

SEQ ID NO: 245 - DNA, 49 bp, synthetic construct
  source 1..49: mol_type = other DNA
gacaagtggccacaaaccaccagactgctcttgtggaaaggacgaaaca

SEQ ID NO: 246 - DNA, 49 bp, synthetic construct
  source 1..49: mol_type = other DNA
gacaagtggccacaaaccaccagctaaggcttgtggaaaggacgaaaca

SEQ ID NO: 247 - DNA, 49 bp, synthetic construct
  source 1..49: mol_type = other DNA
gacaagtggccacaaaccaccagatccaacttgtggaaaggacgaaaca

SEQ ID NO: 248 - DNA, 49 bp, synthetic construct
  source 1..49: mol_type = other DNA
gacaagtggccacaaaccaccaggtgagacttgtggaaaggacgaaaca

SEQ ID NO: 249 - DNA, 49 bp, synthetic construct
  source 1..49: mol_type = other DNA
gacaagtggccacaaaccaccagtaagaccttgtggaaaggacgaaaca

SEQ ID NO: 250 - DNA, 49 bp, synthetic construct
  source 1..49: mol_type = other DNA
gacaagtggccacaaaccaccagcgttagcttgtggaaaggacgaaaca

SEQ ID NO: 251 - DNA, 49 bp, synthetic construct
  source 1..49: mol_type = other DNA
gacaagtggccacaaaccaccaggtgtatcttgtggaaaggacgaaaca

SEQ ID NO: 252 - DNA, 49 bp, synthetic construct
  source 1..49: mol_type = other DNA
gacaagtggccacaaaccaccaggaatgccttgtggaaaggacgaaaca

SEQ ID NO: 253 - DNA, 49 bp, synthetic construct
  source 1..49: mol_type = other DNA
gacaagtggccacaaaccaccagagagtacttgtggaaaggacgaaaca

SEQ ID NO: 254 - DNA, 49 bp, synthetic construct
  source 1..49: mol_type = other DNA
gacaagtggccacaaaccaccagagtccacttgtggaaaggacgaaaca

SEQ ID NO: 255 - DNA, 49 bp, synthetic construct
  source 1..49: mol_type = other DNA
gacaagtggccacaaaccaccagctatggcttgtggaaaggacgaaaca

SEQ ID NO: 256 - DNA, 49 bp, synthetic construct
  source 1..49: mol_type = other DNA
gacaagtggccacaaaccaccagaccttacttgtggaaaggacgaaaca

SEQ ID NO: 257 - DNA, 49 bp, synthetic construct
  source 1..49: mol_type = other DNA
gacaagtggccacaaaccaccagcgccaacttgtggaaaggacgaaaca

SEQ ID NO: 258 - DNA, 21 bp, synthetic construct
  source 1..21: mol_type = other DNA
tctaaagcgcatgctccagac

SEQ ID NO: 259 - DNA, 63 bp, synthetic construct
  source 1..63: mol_type = other DNA
acactctttccctacacgacgctcttccgatcttcgcatagacaagtggccacaaaccaccag

SEQ ID NO: 260 - DNA, 55 bp, synthetic construct
  source 1..55: mol_type = other DNA
gtgactggagttcagacgtgtgctcttccgatcgtctaaagcgcatgctccagac

SEQ ID NO: 261 - DNA, 62 bp, synthetic construct
  source 1..62: mol_type = other DNA
acactctttccctacacgacgctcttccgatctcgcatagacaagtggccacaaaccaccag

SEQ ID NO: 262 - DNA, 56 bp, synthetic construct
  source 1..56: mol_type = other DNA
gtgactggagttcagacgtgtgctcttccgatctgtctaaagcgcatgctccagac

SEQ ID NO: 263 - DNA, 61 bp, synthetic construct
  source 1..61: mol_type = other DNA
acactctttccctacacgacgctcttccgatctgcatagacaagtggccacaaaccaccag

SEQ ID NO: 264 - DNA, 57 bp, synthetic construct
  source 1..57: mol_type = other DNA
gtgactggagttcagacgtgtgctcttccgatcctgtctaaagcgcatgctccagac

SEQ ID NO: 265 - DNA, 60 bp, synthetic construct
  source 1..60: mol_type = other DNA
acactctttccctacacgacgctcttccgatctcatagacaagtggccacaaaccaccag

SEQ ID NO: 266 - DNA, 58 bp, synthetic construct
  source 1..58: mol_type = other DNA
gtgactggagttcagacgtgtgctcttccgatcactgtctaaagcgcatgctccagac

SEQ ID NO: 267 - DNA, 59 bp, synthetic construct
  source 1..59: mol_type = other DNA
acactctttccctacacgacgctcttccgatctatagacaagtggccacaaaccaccag

SEQ ID NO: 268 - DNA, 59 bp, synthetic construct
  source 1..59: mol_type = other DNA
gtgactggagttcagacgtgtgctcttccgatccactgtctaaagcgcatgctccagac

SEQ ID NO: 269 - DNA, 58 bp, synthetic construct
  source 1..58: mol_type = other DNA
acactctttccctacacgacgctcttccgatcttagacaagtggccacaaaccaccag

SEQ ID NO: 270 - DNA, 60 bp, synthetic construct
  source 1..60: mol_type = other DNA
gtgactggagttcagacgtgtgctcttccgatctcactgtctaaagcgcatgctccagac

SEQ ID NO: 271 - DNA, 57 bp, synthetic construct
  source 1..57: mol_type = other DNA
acactctttccctacacgacgctcttccgatctagacaagtggccacaaaccaccag

SEQ ID NO: 272 - DNA, 61 bp, synthetic construct
  source 1..61: mol_type = other DNA
gtgactggagttcagacgtgtgctcttccgatcatcactgtctaaagcgcatgctccagac

SEQ ID NO: 273 - DNA, 56 bp, synthetic construct
  source 1..56: mol_type = other DNA
acactctttccctacacgacgctcttccgatctgacaagtggccacaaaccaccag

SEQ ID NO: 274 - DNA, 62 bp, synthetic construct
  source 1..62: mol_type = other DNA
gtgactggagttcagacgtgtgctcttccgatcgatcactgtctaaagcgcatgctccagac